(19) United States
(12) Patent Application Publication
(10) Pub. No.: US 2018/0171315 A1
(43) Pub. Date: Jun. 21, 2018

(54) POLYPEPTIDES HAVING XANTHAN DEGRADING ACTIVITY AND POLYNUCLEOTIDES ENCODING SAME

(71) Applicant: NOVOZYMES A/S, Bagsvaerd (DK)

(72) Inventors: Dorotea Raventos Segura, Rungsted (DK); Lars Anderson, Malmoe (SE); Lorena Gonzalez Palmen, Malmoe (SE); Liv Spaangner Christiansen, Gentofte (DK); Peter Fischer Hallin, Holte (DK); Leigh Murphy, Roskilde (DK); Mette Louise Dissing Overgaard, Copenhagen S (DK); Rune Nygaard Monrad, Hilleroed (DK); Timothy O'Connell, Landsberg am Lech (DE); Susanne Tondera, Dusseldorf (DE); Nina Mussman, Willich (DE); Daniela Herbst, Dusseldorf (DE)

(73) Assignee: NOVOZYMES A/S, Bagsvaerd (DK)

(21) Appl. No.: 15/736,059

(22) PCT Filed: Sep. 15, 2016
(86) PCT No.: PCT/EP2016/071854

§ 371 Date: Dec. 13, 2017

(30) Foreign Application Priority Data
Sep. 17, 2015 (EP) 15185641.6

(51) Int. Cl. C12N 9/42; C11D 3/386; C09K 8/035

(52) U.S. Cl. CPC C12N 9/2437; C12Y 302/01004; C09K 2208/24; C11D 3/38681; C09K 8/035; C11D 3/38636

(57) ABSTRACT

The present invention relates to polypeptides having xanthan degrading activity, and polynucleotides encoding the polypeptides. The invention also relates to nucleic acid constructs, vectors, and host cells comprising the polynucleotides as well as methods of producing and using the polypeptides.

### REFERENCE TO A SEQUENCE LISTING

This application contains a Sequence Listing in computer readable form, which is incorporated herein by reference.

### BACKGROUND OF THE INVENTION

### Field of the Invention

The present invention relates to polypeptides having xanthan degrading activity. In particular the invention relates to such polypeptides within the glycosyl hydrolase family 5 (GH5) having xanthan degrading activity, and to polynucleotides encoding the polypeptides. The invention also relates to nucleic acid constructs, vectors, and host cells comprising the polynucleotides as well as methods of producing and using the polypeptides.

### Description of the Related Art

Xanthan gum is a polysaccharide secreted by the bacterium Xanthomonas campestris. It is produced by the fermentation of glucose, sucrose, or lactose in an aqueous growth medium by X. campestris. After a fermentation period, the polysaccharide is precipitated from the growth medium with isopropyl alcohol, dried, and ground into a fine powder. Later, the powder is added to a liquid medium to form the gum.

Xanthan is composed of pentasaccharide subunits, forming a cellulose backbone with trisaccharide side chains composed of mannose-(beta1,4)-glucuronic-acid-(beta1,2)-mannose attached to alternate glucose residues in the backbone by alpha1,3 linkages. This biopolymer is of great commercial significance because of its superior pseudoplasticity, thixotropy, and viscosity.

In recent years xanthan gum has been widely used as an ingredient in many consumer products including foods (e.g., as thickening agent in salad dressings and dairy products) and cosmetics (e.g., as stabilizer and thickener in toothpaste and make-up to prevent ingredients from separating) and cosmetics (e.g., sun creams).

In addition, xanthan gum has found use in the oil industry where xanthan gum is used in large quantities to thicken drilling mud. These fluids serve to carry the solids cut by the drilling bit back to the surface. When the circulation stops, the solids still remain suspended in the drilling fluid. The widespread use of horizontal drilling has led to its expanded use. Xanthan gum is also added to self-consolidating concrete, including concrete poured underwater, to increase its viscosity.

The widespread use of xanthan gum has led to a desire to be able to degrade solutions or gels of xanthan gum. Complete enzymatic degradation of xanthan gum has till now required several enzymatic activities including xanthan lyase activity and endo-beta-1,4-glucanase activity. Xanthan lyases are enzymes that cleave the beta-D-mannosylalpha-beta-D-1,4-glucuronosyl bond of xanthan and have been described in the literature. Xanthan degrading enzymes are known in the art e.g., two xanthan lyases isolated from Paenibacillus alginolyticus XL-1 (e.g., Ruijssenaars et al. (1999) ‘A pyruvated mannose-specific xanthan lyase involved in xanthan degradation by Paenibacillus alginolyticus XL-1’, Appl. Environ. Microbiol. 65(6): 2446-2452, and Ruijssenaars et al. (2000), ‘A novel gene encoding xanthan lyase of Paenibacillus alginolyticus strain XL-1’, Appl. Environ. Microbiol. 66(9): 3945-3950).

Glycosyl hydrolases are enzymes that catalyze the hydrolysis of the glycosyl bond to release smaller sugars. There are over 100 classes of Glycosyl hydrolases which have been classified, see Henrissat et al. (1991) ‘A classification of glycosyl hydrolases based on amino-acid sequence similarities’, J. Biochem. 280: 309-316 and the Uniprot website at www.cazy.org. The glycosyl hydrolase family 5 (GH5) includes endo-glucanases (EC 3.2.1.4), endo-beta-1,4-xylanase (EC 3.2.1.8); beta-glucosidase (EC 3.2.1.21); beta-mannosidase (EC 3.2.1.25). However, until now identification of xanthan degrading enzymes have not been reported in glycosyl hydrolase family 5.

The mature peptide in SEQ ID NO: 2 is 45% identical and the mature peptide in SEQ ID NO: 4 is 57% identical to a predicted endoglucanase from the genome of Echinicola vietnamensis (UNIPROT: L0FVA9).

The mature peptide in SEQ ID NO: 6 is 47% identical to an uncharacterized protein from the genome of Barnesiella intestinihominis (UNIPROT: K0WXE1).

The mature peptide in SEQ ID NO: 8 is 100% identical to an uncharacterized protein from the genome of Pseudomonas stutzeri (UNIPROT: M2V1S3).

### SUMMARY OF THE INVENTION

The invention provides new and improved enzymes for the degradation of xanthan gum and the use of such enzymes, such as in the drilling and oil industries.

The present inventors have surprisingly discovered a new group of enzymes that have xanthan degrading activity—and which do not belong to any glycosyl hydrolase family previously known to comprise this enzymatic activity. The enzymes have no significant sequence similarity to any known enzyme having xanthan degrading activity.

The present invention provides polypeptides having xanthan degrading activity, i.e., having activity on xanthan gum and/or having activity on xanthan gum pretreated with xanthan lyase. The present invention further provides polynucleotides encoding the polypeptides.

Accordingly, the present invention provides a polypeptide of glycosyl hydrolase family 5 having xanthan degrading activity. More particularly, the present invention provides a polypeptide of glycosyl hydrolase family 5 having xanthan degrading activity, selected from the group consisting of:

(a) a polypeptide having at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of any of SEQ ID NO: 2, SEQ ID NO: 4, SEQ ID NO: 6 or SEQ ID NO: 8;

(b) a polypeptide encoded by a polynucleotide that hybridizes under medium stringency conditions with (i) the mature polypeptide coding sequence of any of SEQ ID NO: 1, SEQ ID NO: 3, SEQ ID NO: 5 or SEQ ID NO: 7, (ii), or the full-length complement of (i);

(c) a polypeptide encoded by a polynucleotide having at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide coding sequence of any of SEQ ID NO: 1, SEQ ID NO: 3, SEQ ID NO: 5 or SEQ ID NO: 7;

(d) a variant of the mature polypeptide of any of SEQ ID NO: 2 SEQ ID NO: 4, SEQ ID NO: 6 or SEQ ID NO: 8 comprising a substitution, deletion, and/or insertion at one or more positions;

(e) a fragment of the polypeptide of (a), (b), (c), or (d) that has xanthan degrading activity; and

(f) a polypeptide comprising the polypeptide of (a), (b), (c), (d), or (e) and a N-terminal and/or C-terminal His-tag.

The present invention also relates to polynucleotides encoding the polypeptides of the present invention; nucleic acid constructs; recombinant expression vectors; recombinant host cells comprising the polynucleotides; and methods of producing the polypeptides.

The present invention also relates to methods of degrading xanthan gum using the polypeptides, such as in methods for extraction of oil and natural gas, e.g., for controlling viscosity of a drilling fluid or a borehole filtercake.

### Overview of Sequence Listing

SEQ ID NO: 1 is the DNA sequence of the EXa gene as isolated from an Opitutaceae sp.

SEQ ID NO: 2 is the amino acid sequence of the EXa GH5 polypeptide as deduced from SEQ ID NO: 1.

SEQ ID NO: 3 is the DNA sequence of the EXb gene as isolated from an environmental sample

SEQ ID NO: 4 is the amino acid sequence of the EXb GH5 polypeptide as deduced from SEQ ID NO: 3.

SEQ ID NO: 5 is the DNA sequence of the EXc gene as isolated from an environmental sample

SEQ ID NO: 6 is the amino acid sequence of the EXc GH5 polypeptide as deduced from SEQ ID NO: 5.

SEQ ID NO: 7 is the DNA sequence of the EXd gene as obtained from a public database (UNIPROT M2V1S3, originating from a strain of Pseudomonas stutzeri collected from a Galapagos Rift hydrothermal vent, Ecuador).

SEQ ID NO: 8 is the amino acid sequence of the EXd GH5 polypeptide as deduced from SEQ ID NO: 7.

SEQ ID NO:9 is synth codon optimized DNA encoding the EXa GH5 polypeptide.

SEQ ID NO:10 is synth codon optimized DNA encoding the EXb GH5 polypeptide.

SEQ ID NO:11 is synth codon optimized DNA encoding the EXc GH5 polypeptide.

SEQ ID NO:12 is synth codon optimized DNA encoding the EXd GH5 polypeptide.

SEQ ID NO:13 is the EXa GH5 polypeptide+His affinity tag expressed in E. coli.

SEQ ID NO:14 is the EXb GH5 polypeptide+His affinity tag expressed in E. coli.

SEQ ID NO:15 the EXc GH5 polypeptide+His affinity tag expressed in E. coli.

SEQ ID NO:16 is the EXb GH5 polypeptide+His affinity tag expressed in B. subtilis.

SEQ ID NO:17 is the EXc GH5 polypeptide+His affinity tag expressed in B. subtilis.

SEQ ID NO:18 is the EXd GH5 polypeptide+His affinity tag expressed in B. subtilis.

SEQ ID NO:19 is the His affinity tag sequence.

SEQ ID NO:20 is the amino acid sequence of the Bacillus clausii secretion signal.

SEQ ID NO:21 is the amino acid sequence of a xanthan lyase XLa from a Paenibacillus sp (SEQ ID NO: 8 from WO2013167581).

SEQ ID NO:22 is the amino acid sequence of a xanthan lyase XLb from a Paenibacillus sp (SEQ ID NO: 66 from WO2013167581).

SEQ ID NO:23 is the amino acid sequence of a xanthan lyase XLc from a Paenibacillus sp (SEQ ID NO: 68 from WO2013167581).

SEQ ID NO:24 is the amino acid sequence of a xanthan lyase XLd from a Paenibacillus sp (SEQ ID NO: 120 from WO2013167581).

Identity Matrix for mature peptides

| | | | |
|---|---|---|---|
| SEQ ID | SEQ ID | SEQ ID | SEQ ID |
| NO: 2 | NO: 4 | NO: 6 | NO: 8 |
| EXa | EXb | EXc | EXd |

| | | | |
|---|---|---|---|
| SEQ ID NO: 2 | 50 | 71 | 27 |
| EXa | | | |
| SEQ ID NO: 4 | | 47 | 31 |
| EXb | | | |
| SEQ ID NO: 6 | | | 27 |
| EXc | | | |
| SEQ ID NO: 8 | | | |
| EXd | | | |

### DETAILED DESCRIPTION OF THE INVENTION

The present invention provides GH5 polypeptides having xanthan degrading activity and polynucleotides encoding the polypeptides. The polypeptides do not belong to a GH family known to comprise enzymes which degrade xanthan. In addition, the combination of xanthan lyase and an enzyme of the invention having xanthan degrading activity shows a synergistic improved wash performance over using either a xanthan lyase or a GH5 polypeptide having xanthan degrading activity.

### Definitions

Allelic variant: The term “allelic variant” means any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

Catalytic domain: The term “catalytic domain” means the region of an enzyme containing the catalytic machinery of the enzyme.

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Coding sequence: The term “coding sequence” means a polynucleotide, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

Colour clarification: During washing and wearing loose or broken fibers can accumulate on the surface of the fabrics. One consequence can be that the colours of the fabric appear less bright or less intense because of the surface contaminations. Removal of the loose or broken fibers from the textile will partly restore the original colours and looks of the textile. By the term “colour clarification”, as used herein, is meant the partial restoration of the initial colours of textile.

Control sequences: The term “control sequences” means nucleic acid sequences necessary for expression of a polynucleotide encoding a mature polypeptide of the present invention. Each control sequence may be native (i.e., from the same gene) or foreign (i.e., from a different gene) to the polynucleotide encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Detergent Composition: the term “detergent composition” refers to compositions that find use in the removal of undesired compounds from items to be cleaned, such as textiles, dishes, and hard surfaces. The terms encompass any materials/compounds selected for the particular type of cleaning composition desired and the form of the product (e.g., liquid, gel, powder, granulate, paste, or spray compositions) and includes, but is not limited to, detergent compositions (e.g., liquid and/or solid laundry detergents and fine fabric detergents; hard surface cleaning formulations, such as for glass, wood, ceramic and metal counter tops and windows; carpet cleaners; oven cleaners; fabric fresheners; fabric softeners; and textile and laundry pre-spotters, as well as dish wash detergents). In addition to containing an enzyme of the invention, the detergent formulation may contain one or more additional enzymes, and/or components such as surfactants, builders, chelators or chelating agents, bleach system or bleach components, polymers, fabric conditioners, foam boosters, suds suppressors, dyes, perfume, tannish inhibitors, optical brighteners, bactericides, fungicides, soil suspending agents, anti-corrosion agents, enzyme inhibitors or stabilizers, enzyme activators, transferase(s), hydrolytic enzymes, oxido reductases, bluing agents and fluorescent dyes, antioxidants, and solubilizers.

Dish wash: The term “dish wash” refers to all forms of washing dishes, e.g., by hand or automatic dish wash. Washing dishes includes, but is not limited to, the cleaning of all forms of crockery such as plates, cups, glasses, bowls, all forms of cutlery such as spoons, knives, forks and serving utensils as well as ceramics, plastics, metals, china, glass and acrylics.

Dish washing composition: The term “dish washing composition” refers to all forms of compositions for cleaning hard surfaces. The present invention is not restricted to any particular type of dish wash composition or any particular detergent.

Enzyme Detergency benefit: The term “enzyme detergency benefit” is defined herein as the advantageous effect an enzyme may add to a detergent compared to the same detergent without the enzyme. Important detergency benefits which can be provided by enzymes are stain removal with no or very little visible soils after washing and or cleaning, prevention or reduction of redeposition of soils released in the washing process an effect that also is termed anti-redeposition, restoring fully or partly the whiteness of textiles, which originally were white but after repeated use and wash have obtained a greyish or yellowish appearance an effect that also is termed whitening. Textile care benefits, which are not directly related to catalytic stain removal or prevention of redeposition of soils are also important for enzyme detergency benefits. Examples of such textile care benefits are prevention or reduction of dye transfer from one fabric to another fabric or another part of the same fabric an effect that is also termed dye transfer inhibition or anti-backstaining, removal of protruding or broken fibers from a fabric surface to decrease pilling tendencies or remove already existing pills or fuzz an effect that also is termed anti-pilling, improvement of the fabric-softness, colour clarification of the fabric and removal of particulate soils which are trapped in the fibers of the fabric or garment. Enzymatic bleaching is a further enzyme detergency benefit where the catalytic activity generally is used to catalyze the formation of bleaching component such as hydrogen peroxide or other peroxides.

Expression: The term “expression” includes any step involved in the production of a polypeptide including, but not limited to, transcription, post-transcriptional modification, translation, post-translational modification, and secretion.

Expression vector: The term “expression vector” means a linear or circular DNA molecule that comprises a polynucleotide encoding a polypeptide and is operably linked to control sequences that provide for its expression.

Fragment: The term “fragment” means a polypeptide having one or more (e.g., several) amino acids absent from the amino and/or carboxyl terminus of a mature polypeptide or domain; wherein the fragment has xanthan degrading activity.

Hard surface cleaning: The term “Hard surface cleaning” is defined herein as cleaning of hard surfaces wherein hard surfaces may include floors, tables, walls, roofs etc. as well as surfaces of hard objects such as cars (car wash) and dishes (dish wash). Dish washing includes but are not limited to cleaning of plates, cups, glasses, bowls, and cutlery such as spoons, knives, forks, serving utensils, ceramics, plastics, metals, china, glass and acrylics.

Host cell: The term “host cell” means any cell type that is susceptible to transformation, transfection, transduction, or the like with a nucleic acid construct or expression vector comprising a GH5 polynucleotide of the present invention. The term “host cell” encompasses any progeny of a parent cell that is not identical to the parent cell due to mutations that occur during replication.

Improved wash performance: The term “improved wash performance” is defined herein as a (variant) enzyme (also a blend of enzymes, not necessarily only variants but also backbones, and in combination with certain cleaning composition etc.) displaying an alteration of the wash performance of a protease variant relative to the wash performance of the parent protease variant e.g. by increased stain removal. The term “wash performance” includes wash performance in laundry but also e.g. in dish wash.

Isolated: The term “isolated” means a substance in a form or environment that does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., recombinant production in a host cell; multiple copies of a gene encoding the substance; and use of a stronger promoter than the promoter naturally associated with the gene encoding the substance). An isolated substance may be present in a fermentation broth sample; e.g. a host cell may be genetically modified to express the polypeptide of the invention. The fermentation broth from that host cell will comprise the isolated polypeptide.

Mature polypeptide: The term “mature polypeptide” means a polypeptide in its final form following translation and any post-translational modifications, such as N-terminal processing, C-terminal truncation, glycosylation, phosphorylation, etc. In one aspect, the mature polypeptide is amino acids 1 to 802 of SEQ ID NO: 2. In a second aspect, the mature polypeptide is amino acids 1 to 808 of SEQ ID NO: 4. In a third aspect, the mature polypeptide is amino acids 1 to 800 of SEQ ID NO: 6. In a fourth aspect, the mature polypeptide is amino acids 1 to 657 of SEQ ID NO: 8. It is known in the art that a host cell may produce a mixture of two of more different mature polypeptides (i.e., with a different C-terminal and/or N-terminal amino acid) expressed by the same polynucleotide. It is also known in the art that different host cells process polypeptides differently, and thus, one host cell expressing a polynucleotide may produce a different mature polypeptide (e.g., having a different C-terminal and/or N-terminal amino acid) as compared to another host cell expressing the same polynucleotide.

Mature polypeptide coding sequence: The term “mature polypeptide coding sequence” means a polynucleotide that encodes a mature polypeptide having xanthan degrading activity. In one aspect, the mature polypeptide coding sequence is nucleotides 109 to 2514 of SEQ ID NO: 1. Nucleotides 1 to 108 of SEQ ID NO: 1 encode a signal peptide. In one aspect, the mature polypeptide coding sequence is nucleotides 112 to 2493 of SEQ ID NO: 3. Nucleotides 1 to 111 of SEQ ID NO: 3 encode a signal peptide. In one aspect, the mature polypeptide coding sequence is nucleotides 106 to 2505 of SEQ ID NO: 5. Nucleotides 1 to 105 of SEQ ID NO: 5 encode a signal peptide. In one aspect, the mature polypeptide coding sequence is nucleotides 109 to 2079 of SEQ ID NO: 7. Nucleotides 1 to 108 of SEQ ID NO: 7 encode a signal peptide.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” means a configuration in which a control sequence is placed at an appropriate position relative to the coding sequence of a polynucleotide such that the control sequence directs expression of the coding sequence.

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the −nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the −nobrief option) is used as the percent identity and is calculated as follows:

(Identical Deoxyribonucleotides×100)/(Length of Alignment−Total Number of Gaps in Alignment)

Stringency conditions: The term “very low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 2×SSC, 0.2% SDS at 45° C.

The term “low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 2×SSC, 0.2% SDS at 50° C.

The term “medium stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 2×SSC, 0.2% SDS at 55° C.

The term “medium-high stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 2×SSC, 0.2% SDS at 60° C.

The term “high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 2×SSC, 0.2% SDS at 65° C.

The term “very high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 2×SSC, 0.2% SDS at 70° C.

Subsequence: The term “subsequence” means a polynucleotide having one or more (e.g., several) nucleotides absent from the 5′ and/or 3′ end of a mature polypeptide coding sequence; wherein the subsequence encodes a fragment having xanthan degrading activity.

Textile: The term “textile” means any textile material including yarns, yarn intermediates, fibers, non-woven materials, natural materials, synthetic materials, and any other textile material, fabrics made of these materials and products made from fabrics (e.g., garments and other articles). The textile or fabric may be in the form of knits, wovens, denims, non-wovens, felts, yarns, and towelling. The textile may be cellulose based such as natural cellulosics, including cotton, flax/linen, jute, ramie, sisal or coir or manmade cellulosics (e.g. originating from wood pulp) including viscose/rayon, ramie, cellulose acetate fibers (tricell), lyocell or blends thereof. The textile or fabric may also be non-cellulose based such as natural polyamides including wool, camel, cashmere, mohair, rabit and silk or synthetic polymer such as nylon, aramid, polyester, acrylic, polypropylen and spandex/elastane, or blends thereof as well as blend of cellulose based and non-cellulose based fibers. Examples of blends are blends of cotton and/or rayon/viscose with one or more companion material such as wool, synthetic fibers (e.g. polyamide fibers, acrylic fibers, polyester fibers, polyvinyl alcohol fibers, polyvinyl chloride fibers, polyurethane fibers, polyurea fibers, aramid fibers), and cellulose-containing fibers (e.g. rayon/viscose, ramie, flax/linen, jute, cellulose acetate fibers, lyocell). Fabric may be conventional washable laundry, for example stained household laundry. When the term fabric or garment is used it is intended to include the broader term textiles as well.

Textile care benefit: “Textile care benefits”, which are not directly related to catalytic stain removal or prevention of redeposition of soils, are also important for enzyme detergency benefits. Examples of such textile care benefits are prevention or reduction of dye transfer from one textile to another textile or another part of the same textile an effect that is also termed dye transfer inhibition or anti-backstaining, removal of protruding or broken fibers from a textile surface to decrease pilling tendencies or remove already existing pills or fuzz an effect that also is termed anti-pilling, improvement of the textile-softness, colour clarification of the textile and removal of particulate soils which are trapped in the fibers of the textile. Enzymatic bleaching is a further enzyme detergency benefit where the catalytic activity generally is used to catalyze the formation of bleaching component such as hydrogen peroxide or other peroxides or other bleaching species.

Wash performance: The term “wash performance” is used as an enzyme's ability to remove stains present on the object to be cleaned during e.g. wash or hard surface cleaning. The improvement in the wash performance may be quantified by calculating the so-called intensity value (Int) as defined in ‘Automatic Mechanical Stress Assay (AMSA) for laundry’ herein. See also the wash performance test in Example 18 herein.

Whiteness: The term “Whiteness” is defined herein as a broad term with different meanings in different regions and for different customers. Loss of whiteness can e.g. be due to greying, yellowing, or removal of optical brighteners/hueing agents. Greying and yellowing can be due to soil redeposition, body soils, colouring from e.g. iron and copper ions or dye transfer. Whiteness might include one or several issues from the list below: colorant or dye effects; incomplete stain removal (e.g. body soils, sebum etc.); re-deposition (greying, yellowing or other discolorations of the object) (removed soils re-associates with other part of textile, soiled or unsoiled); chemical changes in textile during application; and clarification or brightening of colours.

Xanthan Lyase: The term “xanthan lyase” is defined herein as an enzyme that cleaves the beta-D-mannosyl-beta-D-1,4-glucuronosyl bonds in xanthan gum (EC 4.2.2.12). For purposes of the present invention, xanthan lyase activity is determined according to the procedure described in the Examples in the ‘Xanthan lyase activity assay.

Xanthan degrading activity: The term “xanthan degrading activity” is defined herein as ability to cause viscosity reduction of a xanthan solution. Xanthan solution is highly viscous even at low polymer concentrations, and this viscosity is associated with the polymer degree of xanthan. Therefore, viscosity reduction can be used to monitor xanthan degradation. The viscosity reduction may be detected using the viscosity pressure assay described in Example 6.

Xanthan degrading activity includes activity towards intact xanthan as well as activity towards xanthan pretreated with xanthan lyase (modified xanthan gum—see Example 8).

Activity on xanthan gum: The term “GH5 polypeptide having activity on xanthan gum” or a “polypeptide having activity on xanthan gum and belonging to the GH5 class of glycosyl hydrolases” is defined as a polypeptide comprising a domain belonging to the GH5 class of glycosyl hydrolases, and having significant activity on xanthan gum. In one aspect of the invention a GH5 polypeptide having activity on xanthan gum may be a polypeptide having a sequence selected among SEQ ID NO: 2, SEQ ID NO: 4, SEQ ID NO: 6, and SEQ ID NO: 8.

Activity on xanthan gum pretreated with xanthan lyase: The term “GH5 polypeptide having activity on xanthan gum pretreated with xanthan lyase” or a “polypeptide having activity on xanthan gum pretreated with xanthan lyase and belonging to the GH5 class of glycosyl hydrolases” is defined as a polypeptide comprising a domain belonging to the GH5 class of glycosyl hydrolases, and having significant activity on xanthan gum pretreated with xanthan lyase (modified xanthan gum—see Example 8). In one aspect of the invention a GH5 polypeptide having activity on xanthan gum pretreated with xanthan lyase may be a polypeptide having a sequence selected among SEQ ID NO: 2, SEQ ID NO: 4, SEQ ID NO: 6, and SEQ ID NO: 8.

### Polypeptides Having Xanthan Degrading Activity

In an embodiment, the present invention relates to polypeptides having a sequence identity to the mature polypeptide of any of SEQ ID NO: 2, 4, 6 and 8 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, which have xanthan degrading activity. In one aspect, the polypeptides differ by up to 10 amino acids, e.g., 1, 2, 3, 4, 5, 6, 7, 8, 9, or 10, from the mature polypeptide of any of SEQ ID NO: 2, 4, 6 and 8.

In a particular embodiment the invention relates to polypeptides having a sequence identity to the mature polypeptide of any of SEQ ID NO: 2, 4, 6 and 8 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 70% of the xanthan degrading activity of the mature polypeptide of any of SEQ ID NO: 2, 4, 6 and 8.

In a particular embodiment the invention relates to polypeptides having a sequence identity to the mature polypeptide of any of SEQ ID NO: 2, 4, 6 and 8 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 75% of the xanthan degrading activity of the mature polypeptide of any of SEQ ID NO: 2, 4, 6 and 8.

In a particular embodiment the invention relates to polypeptides having a sequence identity to the mature polypeptide of any of SEQ ID NO: 2, 4, 6 and 8 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 80% of the xanthan degrading activity of the mature polypeptide of any of SEQ ID NO: 2, 4, 6 and 8.

In a particular embodiment the invention relates to polypeptides having a sequence identity to the mature polypeptide of any of SEQ ID NO: 2, 4, 6 and 8 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 85% of the xanthan degrading activity of the mature polypeptide of any of SEQ ID NO: 2, 4, 6 and 8.

In a particular embodiment the invention relates to polypeptides having a sequence identity to the mature polypeptide of any of SEQ ID NO: 2, 4, 6 and 8 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 90% of the xanthan degrading activity of the mature polypeptide of any of SEQ ID NO: 2, 4, 6 and 8.

In a particular embodiment the invention relates to polypeptides having a sequence identity to the mature polypeptide of any of SEQ ID NO: 2, 4, 6 and 8 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 95% of the xanthan degrading activity of the mature polypeptide of any of SEQ ID NO: 2, 4, 6 and 8.

In a particular embodiment the invention relates to polypeptides having a sequence identity to the mature polypeptide of any of SEQ ID NO: 2, 4, 6 and 8 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 100% of the xanthan degrading activity of the mature polypeptide of any of SEQ ID NO: 2, 4, 6 and 8.

In an embodiment, the polypeptide has been isolated. A polypeptide of the present invention preferably comprises or consists of the amino acid sequence of any of SEQ ID NO: 2, 4, 6 and 8 or an allelic variant thereof; or is a fragment thereof having xanthan degrading activity. In another aspect, the polypeptide comprises or consists of the mature polypeptide of any of SEQ ID NO: 2, 4, 6 and 8. In another aspect, the polypeptide comprises or consists of amino acids 1 to 802 of SEQ ID NO: 2, amino acids 1 to 808 of SEQ ID NO: 4, amino acids 1 to 800 of SEQ ID NO: 6, or amino acids 1 to 657 of SEQ ID NO: 8.

In another embodiment, the present invention relates to a polypeptide having xanthan degrading activity encoded by a polynucleotide that hybridizes under very low stringency conditions, low stringency conditions, medium stringency conditions, medium-high stringency conditions, high stringency conditions, or very high stringency conditions with (i) the mature polypeptide coding sequence of SEQ ID NO: 1, (ii), or (iii) the full-length complement of (i) or (ii) (Sambrook et al., 1989, Molecular Cloning, A Laboratory Manual, 2d edition, Cold Spring Harbor, New York). In an embodiment, the polypeptide has been isolated.

The polynucleotide of any of SEQ ID NO: 1, 3, 5, or 7 or a subsequence thereof, as well as the polypeptide of any of SEQ ID NO: 2, 4, 6 and 8 or a fragment thereof may be used to design nucleic acid probes to identify and clone DNA encoding polypeptides having xanthan degrading activity from strains of different genera or species according to methods well known in the art. In particular, such probes can be used for hybridization with the genomic DNA or cDNA of a cell of interest, following standard Southern blotting procedures, in order to identify and isolate the corresponding gene therein. Such probes can be considerably shorter than the entire sequence, but should be at least 15, e.g., at least 25, at least 35, or at least 70 nucleotides in length. Preferably, the nucleic acid probe is at least 100 nucleotides in length, e.g., at least 200 nucleotides, at least 300 nucleotides, at least 400 nucleotides, at least 500 nucleotides, at least 600 nucleotides, at least 700 nucleotides, at least 800 nucleotides, or at least 900 nucleotides in length. Both DNA and RNA probes can be used. The probes are typically labeled for detecting the corresponding gene (for example, with ³²P, ³H, ³⁵S, biotin, or avidin). Such probes are encompassed by the present invention.

A genomic DNA or cDNA library prepared from such other strains may be screened for DNA that hybridizes with the probes described above and encodes a polypeptide having xanthan degrading activity. Genomic or other DNA from such other strains may be separated by agarose or polyacrylamide gel electrophoresis, or other separation techniques. DNA from the libraries or the separated DNA may be transferred to and immobilized on nitrocellulose or other suitable carrier material. In order to identify a clone or DNA that hybridizes with SEQ ID NO: 1 or a subsequence thereof, the carrier material is used in a Southern blot.

For purposes of the present invention, hybridization indicates that the polynucleotide hybridizes to a labeled nucleic acid probe corresponding to (i) any of SEQ ID NO: 1, 3, 5, or 7; (ii) the mature polypeptide coding sequence of any of SEQ ID NO: 1, 3, 5, or 7; (iii) the full-length complement thereof; or (iv) a subsequence thereof; under very low to very high stringency conditions. Molecules to which the nucleic acid probe hybridizes under these conditions can be detected using, for example, X-ray film or any other detection means known in the art.

In another embodiment, the present invention relates to a polypeptide having xanthan degrading activity encoded by a polynucleotide having a sequence identity to the mature polypeptide coding sequence of any of SEQ ID NO: 1, 3, 5, or 7 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In a further embodiment, the polypeptide has been isolated.

In another embodiment, the present invention relates to variants of the mature polypeptide of any of SEQ ID NO: 2, 4, 6 and 8 comprising a substitution, deletion, and/or insertion at one or more (e.g., several) positions. In an embodiment, the number of amino acid substitutions, deletions and/or insertions introduced into the mature polypeptide of any of SEQ ID NO: 2, 4, 6 and 8 is up to 10, e.g., 1, 2, 3, 4, 5, 6, 7, 8, 9, or 10. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tag, an antigenic epitope or a binding domain. SEQ ID NO: 13, 14 and 15 show the polypeptides of the invention (SEQ ID NO: 2, 4 and 6) with an N-terminal poly histidine tag (His-tag). SEQ ID NO: 16, 17 and 18 show the polypeptides of the invention (SEQ ID NO: 4, 6 and 8) with an N-terminal poly histidine tag.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

### Sources of Polypeptides Having Xanthan Degrading Activity

A polypeptide having xanthan degrading activity of the present invention may be obtained from microorganisms of any genus. For purposes of the present invention, the term “obtained from” as used herein in connection with a given source shall mean that the polypeptide encoded by a polynucleotide is produced by the source or by a strain in which the polynucleotide from the source has been inserted.

In an aspect, the polypeptide is a polypeptide obtained from an Opitutaceae species.

The polypeptide may be identified and obtained from other sources including microorganisms isolated from nature (e.g., soil, composts, water, etc.) or DNA samples obtained directly from natural materials (e.g., soil, composts, water, etc.) using the above-mentioned probes. Techniques for isolating microorganisms and DNA directly from natural habitats are well known in the art. A polynucleotide encoding the polypeptide may then be obtained by similarly screening a genomic DNA or cDNA library of another microorganism or mixed DNA sample. Once a polynucleotide encoding a polypeptide has been detected with the probe(s), the polynucleotide can be isolated or cloned by utilizing techniques that are known to those of ordinary skill in the art (see, e.g., Sambrook et al., 1989, supra).

### Polynucleotides

The present invention also relates to polynucleotides encoding a polypeptide, as described herein. In an embodiment, the polynucleotide encoding the polypeptide of the present invention has been isolated.

The techniques used to isolate or clone a polynucleotide are known in the art and include isolation from genomic DNA or cDNA, or a combination thereof. The cloning of the polynucleotides from genomic DNA can be effected, e.g., by using the well-known polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligation activated transcription (LAT) and polynucleotide-based amplification (NASBA) may be used. The polynucleotides may be cloned from a strain of an Opitutaceae species, or a related organism and thus, for example, may be an allelic or species variant of the polypeptide encoding region of the polynucleotide.

Modification of a polynucleotide encoding a polypeptide of the present invention may be necessary for synthesizing polypeptides substantially similar to the polypeptide. The term “substantially similar” to the polypeptide refers to non-naturally occurring forms of the polypeptide.

### Nucleic Acid Constructs

The present invention also relates to nucleic acid constructs comprising a GH5 polynucleotide of the present invention operably linked to one or more control sequences that direct the expression of the coding sequence in a suitable host cell under conditions compatible with the control sequences.

The polynucleotide may be manipulated in a variety of ways to provide for expression of the polypeptide. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide that is recognized by a host cell for expression of a polynucleotide encoding a polypeptide of the present invention. The promoter contains transcriptional control sequences that mediate the expression of the polypeptide. The promoter may be any polynucleotide that shows transcriptional activity in the host cell including variant, truncated, and hybrid promoters, and may be obtained from genes encoding extracellular or intracellular polypeptides either homologous or heterologous to the host cell.

Examples of suitable promoters for directing transcription of the nucleic acid constructs of the present invention in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xylA and xylB genes, Bacillus thuringiensis cryIIIA gene (Agaisse and Lereclus, 1994, Molecular Microbiology 13: 97-107), E. coli lac operon, E. coli trc promoter (Egon et al., 1988, Gene 69: 301-315), Streptomyces coelicolor agarase gene (dagA), and prokaryotic beta-lactamase gene (Villa-Kamaroff et al., 1978, Proc. Natl. Acad. Sci. USA 75: 3727-3731), as well as the tac promoter (DeBoer et al., 1983, Proc. Natl. Acad. Sci. USA 80: 21-25). Further promoters are described in “Useful proteins from recombinant bacteria” in Gilbert et al., 1980, Scientific American 242: 74-94; and in Sambrook et al., 1989, supra. Examples of tandem promoters are disclosed in WO 99/43835.

Examples of suitable promoters for directing transcription of the nucleic acid constructs of the present invention in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Daria (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor, as well as the NA2-tpi promoter (a modified promoter from an Aspergillus neutral alpha-amylase gene in which the untranslated leader has been replaced by an untranslated leader from an Aspergillus triose phosphate isomerase gene; non-limiting examples include modified promoters from an Aspergillus niger neutral alpha-amylase gene in which the untranslated leader has been replaced by an untranslated leader from an Aspergillus nidulans or Aspergillus oryzae triose phosphate isomerase gene); and variant, truncated, and hybrid promoters thereof. Other promoters are described in U.S. Pat. No. 6,011,147.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8: 423-488.

The control sequence may also be a transcription terminator, which is recognized by a host cell to terminate transcription. The terminator is operably linked to the 3′-terminus of the polynucleotide encoding the polypeptide. Any terminator that is functional in the host cell may be used in the present invention.

Preferred terminators for bacterial host cells are obtained from the genes for Bacillus clausii alkaline protease (aprH), Bacillus licheniformis alpha-amylase (amyL), and Escherichia coli ribosomal RNA (rrnB).

Preferred terminators for filamentous fungal host cells are obtained from the genes for Aspergillus nidulans acetamidase, Aspergillus nidulans anthranilate synthase, Aspergillus niger glucoamylase, Aspergillus niger alpha-glucosidase, Aspergillus oryzae TAKA amylase, Fusarium oxysporum trypsin-like protease, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIIA gene (WO 94/25612) and a Bacillus subtilis SP82 gene (Hue et al., 1995, Journal of Bacteriology 177: 3465-3471).

The control sequence may also be a leader, a nontranslated region of an mRNA that is important for translation by the host cell. The leader is operably linked to the 5′-terminus of the polynucleotide encoding the polypeptide. Any leader that is functional in the host cell may be used.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′-terminus of the polynucleotide and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase Aspergillus oryzae TAKA amylase, and Fusarium oxysporum trypsin-like protease.

Useful polyadenylation sequences for yeast host cells are described by Guo and Sherman, 1995, Mol. Cellular Biol. 15: 5983-5990.

The control sequence may also be a signal peptide coding region that encodes a signal peptide linked to the N-terminus of a polypeptide and directs the polypeptide into the cell's secretory pathway. The 5′-end of the coding sequence of the polynucleotide may inherently contain a signal peptide coding sequence naturally linked in translation reading frame with the segment of the coding sequence that encodes the polypeptide. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the polypeptide. However, any signal peptide coding sequence that directs the expressed polypeptide into the secretory pathway of a host cell may be used.

Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Simonen and Palva, 1993, Microbiological Reviews 57: 109-137.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

The control sequence may also be a propeptide coding sequence that encodes a propeptide positioned at the N-terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a polypeptide and the signal peptide sequence is positioned next to the N-terminus of the propeptide sequence.

It may also be desirable to add regulatory sequences that regulate expression of the polypeptide relative to the growth of the host cell. Examples of regulatory sequences are those that cause expression of the gene to be turned on or off in response to a chemical or physical stimulus, including the presence of a regulatory compound. Regulatory sequences in prokaryotic systems include the lac, tac, and trp operator systems. In yeast, the ADH2 system or GAL1 system may be used. In filamentous fungi, the Aspergillus niger glucoamylase promoter, Aspergillus oryzae TAKA alpha-amylase promoter, and Aspergillus oryzae glucoamylase promoter, Trichoderma reesei cellobiohydrolase I promoter, and Trichoderma reesei cellobiohydrolase II promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the polypeptide would be operably linked to the regulatory sequence.

### Expression Vectors

The present invention also relates to recombinant expression vectors comprising a GH5 polynucleotide of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the polypeptide at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are Bacillus licheniformis or Bacillus subtilis dal genes, or markers that confer antibiotic resistance such as ampicillin, chloramphenicol, kanamycin, neomycin, spectinomycin, or tetracycline resistance. Suitable markers for yeast host cells include, but are not limited to, ADE2, HIS3, LEU2, LYS2, MET3, TRP1, and URA3. Selectable markers for use in a filamentous fungal host cell include, but are not limited to, adeA (phosphoribosylaminoimidazole-succinocarboxamide synthase), adeB (phosphoribosyl-aminoimidazole synthase), amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are Aspergillus nidulans or Aspergillus oryzae amdS and pyrG genes and a Streptomyces hygroscopicus bar gene. Preferred for use in a Trichoderma cell are adeA, adeB, amdS, hph, and pyrG genes.

The selectable marker may be a dual selectable marker system as described in WO 2010/039889. In one aspect, the dual selectable marker is an hph-tk dual selectable marker system.

The vector preferably contains an element(s) that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMβ1 permitting replication in Bacillus.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANS1 (Gems et al., 1991, Gene 98: 61-67; Cullen et al., 1987, Nucleic Acids Res. 15: 9163-9175; WO 00/24883). Isolation of the AMA1 gene and construction of plasmids or vectors comprising the gene can be accomplished according to the methods disclosed in WO 00/24883.

More than one copy of a GH5 polynucleotide of the present invention may be inserted into a host cell to increase production of a polypeptide. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Host Cells

The present invention also relates to recombinant host cells, comprising a GH5 polynucleotide of the present invention operably linked to one or more control sequences that direct the production of a polypeptide of the present invention. A construct or vector comprising a polynucleotide is introduced into a host cell so that the construct or vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the gene encoding the polypeptide and its source.

The host cell may be any cell useful in the recombinant production of a polypeptide of the present invention, e.g., a prokaryote or a eukaryote.

The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma.

The bacterial host cell may be any Bacillus cell including, but not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells.

The bacterial host cell may also be any Streptococcus cell including, but not limited to, Streptococcus equisimilis, Streptococcus pyogenes, Streptococcus uberis, and Streptococcus equi subsp. Zooepidemicus cells.

The bacterial host cell may also be any Streptomyces cell including, but not limited to, Streptomyces achromogenes, Streptomyces avermitilis, Streptomyces coelicolor, Streptomyces griseus, and Streptomyces lividans cells.

The introduction of DNA into a Bacillus cell may be effected by protoplast transformation (see, e.g., Chang and Cohen, 1979, Mol. Gen. Genet. 168: 111-115), competent cell transformation (see, e.g., Young and Spizizen, 1961, J. Bacteriol. 81: 823-829, or Dubnau and Davidoff-Abelson, 1971, J. Mol. Biol. 56: 209-221), electroporation (see, e.g., Shigekawa and Dower, 1988, Biotechniques 6: 742-751), or conjugation (see, e.g., Koehler and Thorne, 1987, J. Bacteriol. 169: 5271-5278). The introduction of DNA into an E. coli cell may be effected by protoplast transformation (see, e.g., Hanahan, 1983, J. Mol. Biol. 166: 557-580) or electroporation (see, e.g., Dower et al., 1988, Nucleic Acids Res. 16: 6127-6145). The introduction of DNA into a Streptomyces cell may be effected by protoplast transformation, electroporation (see, e.g., Gong et al., 2004, Folia Microbiol. (Praha) 49: 399-405), conjugation (see, e.g., Mazodier et al., 1989, J. Bacteriol. 171: 3583-3585), or transduction (see, e.g., Burke et al., 2001, Proc. Natl. Acad. Sci. USA 98: 6289-6294). The introduction of DNA into a Pseudomonas cell may be effected by electroporation (see, e.g., Choi et al., 2006, J. Microbiol. Methods 64: 391-397) or conjugation (see, e.g., Pinedo and Smets, 2005, Appl. Environ. Microbiol. 71: 51-57). The introduction of DNA into a Streptococcus cell may be effected by natural competence (see, e.g., Perry and Kuramitsu, 1981, Infect. Immun. 32: 1295-1297), protoplast transformation (see, e.g., Catt and Jollick, 1991, Microbios 68: 189-207), electroporation (see, e.g., Buckley et al., 1999, Appl. Environ. Microbiol. 65: 3800-3804), or conjugation (see, e.g., Clewell, 1981, Microbiol. Rev. 45: 409-436). However, any method known in the art for introducing DNA into a host cell can be used.

The host cell may also be a eukaryote, such as a mammalian, insect, plant, or fungal cell.

The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

The fungal host cell may be a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, Passmore, and Davenport, editors, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell, such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81: 1470-1474, and Christensen et al., 1988, Bio/Technology 6: 1419-1422. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, J. Bacteriol. 153: 163; and Hinnen et al., 1978, Proc. Natl. Acad. Sci. USA 75: 1920.

### Methods of Production

The present invention also relates to methods of producing a polypeptide of the present invention, comprising (a) cultivating a cell, which in its wild-type form produces the polypeptide, under conditions conducive for production of the polypeptide; and optionally, (b) recovering the polypeptide. In one aspect, the cell is an Opitutaceae species cell.

The present invention also relates to methods of producing a polypeptide of the present invention, comprising (a) cultivating a recombinant host cell of the present invention under conditions conducive for production of the polypeptide; and optionally, (b) recovering the polypeptide.

The host cells are cultivated in a nutrient medium suitable for production of the polypeptide using methods known in the art. For example, the cells may be cultivated by shake flask cultivation, or small-scale or large-scale fermentation (including continuous, batch, fed-batch, or solid state fermentations) in laboratory or industrial fermentors in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The polypeptide may be detected using methods known in the art that are specific for the polypeptides. These detection methods include, but are not limited to, use of specific antibodies, formation of an enzyme product, or disappearance of an enzyme substrate. For example, an enzyme assay may be used to determine the activity of the polypeptide.

The polypeptide may be recovered using methods known in the art. For example, the polypeptide may be recovered from the nutrient medium by conventional procedures including, but not limited to, collection, centrifugation, filtration, extraction, spray-drying, evaporation, or precipitation. In one aspect, a fermentation broth comprising the polypeptide is recovered.

The polypeptide may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure polypeptides.

In an alternative aspect, the polypeptide is not recovered, but rather a host cell of the present invention expressing the polypeptide is used as a source of the polypeptide.

### Fermentation Broth Formulations or Cell Compositions

The present invention also relates to a fermentation broth formulation or a cell composition comprising a polypeptide of the present invention. The fermentation broth product further comprises additional ingredients used in the fermentation process, such as, for example, cells (including, the host cells containing the gene encoding the polypeptide of the present invention which are used to produce the polypeptide of interest), cell debris, biomass, fermentation media and/or fermentation products. In some embodiments, the composition is a cell-killed whole broth containing organic acid(s), killed cells and/or cell debris, and culture medium.

The term “fermentation broth” as used herein refers to a preparation produced by cellular fermentation that undergoes no or minimal recovery and/or purification. For example, fermentation broths are produced when microbial cultures are grown to saturation, incubated under carbon-limiting conditions to allow protein synthesis (e.g., expression of enzymes by host cells) and secretion into cell culture medium. The fermentation broth can contain unfractionated or fractionated contents of the fermentation materials derived at the end of the fermentation.

Typically, the fermentation broth is unfractionated and comprises the spent culture medium and cell debris present after the microbial cells (e.g., filamentous fungal cells) are removed, e.g., by centrifugation. In some embodiments, the fermentation broth contains spent cell culture medium, extracellular enzymes, and viable and/or nonviable microbial cells.

In an embodiment, the fermentation broth formulation and cell compositions comprise a first organic acid component comprising at least one 1-5 carbon organic acid and/or a salt thereof and a second organic acid component comprising at least one 6 or more carbon organic acid and/or a salt thereof. In a specific embodiment, the first organic acid component is acetic acid, formic acid, propionic acid, a salt thereof, or a mixture of two or more of the foregoing and the second organic acid component is benzoic acid, cyclohexanecarboxylic acid, 4-methylvaleric acid, phenylacetic acid, a salt thereof, or a mixture of two or more of the foregoing.

In one aspect, the composition contains an organic acid(s), and optionally further contains killed cells and/or cell debris. In one embodiment, the killed cells and/or cell debris are removed from a cell-killed whole broth to provide a composition that is free of these components.

The fermentation broth formulations or cell compositions may further comprise a preservative and/or anti-microbial (e.g., bacteriostatic) agent, including, but not limited to, sorbitol, sodium chloride, potassium sorbate, and others known in the art.

The cell-killed whole broth or composition may contain the unfractionated contents of the fermentation materials derived at the end of the fermentation. Typically, the cell-killed whole broth or composition contains the spent culture medium and cell debris present after the microbial cells (e.g., filamentous fungal cells) are grown to saturation, incubated under carbon-limiting conditions to allow protein synthesis. In some embodiments, the cell-killed whole broth or composition contains the spent cell culture medium, extracellular enzymes, and killed filamentous fungal cells. In some embodiments, the microbial cells present in the cell-killed whole broth or composition can be permeabilized and/or lysed using methods known in the art.

A whole broth or cell composition as described herein is typically a liquid, but may contain insoluble components, such as killed cells, cell debris, culture media components, and/or insoluble enzyme(s). In some embodiments, insoluble components may be removed to provide a clarified liquid composition.

The whole broth formulations and cell compositions of the present invention may be produced by a method described in WO 90/15861 or WO 2010/096673.

### Detergent Composition

The polypeptide of the present invention may be added to a detergent composition in an amount corresponding to 0.0001-200 mg of enzyme protein, such as 0.0005-100 mg of enzyme protein, preferably 0.001-30 mg of enzyme protein, more preferably 0.005-8 mg of enzyme protein, even more preferably 0.01-2 mg of enzyme protein per litre of wash liquor.

A composition for use in automatic dishwash (ADW), for example, may include 0.0001%-50%, such as 0.001%-20%, such as 0.01%-10%, such as 0.05-5% of enzyme protein by weight of the composition.

A composition for use in laundry powder, for example, may include 0.0001%-50%, such as 0.001%-20%, such as 0.01%-10%, such as 0.05%-5% of enzyme protein by weight of the composition.

A composition for use in laundry liquid, for example, may include 0.0001%-10%, such as 0.001-7%, such as 0.1%-5% of enzyme protein by weight of the composition.

The enzyme(s) of the detergent composition may be stabilized using conventional stabilizing agents, e.g., a polyol such as propylene glycol or glycerol, a sugar or sugar alcohol, lactic acid, boric acid, or a boric acid derivative, e.g., an aromatic borate ester, or a phenyl boronic acid derivative such as 4-formylphenyl boronic acid, and the composition may be formulated as described in, for example, WO92/19709 and WO92/19708.

In certain markets different wash conditions and, as such, different types of detergents are used. This is disclosed in e.g. EP 1 025 240. For example, In Asia (Japan) a low detergent concentration system is used, while the United States uses a medium detergent concentration system, and Europe uses a high detergent concentration system.

A detergent composition may comprise an enzyme of the present invention in combination with one or more additional cleaning composition components. The choice of additional components is within the skill of the artisan and includes conventional ingredients, including the exemplary non-limiting components set forth below.

The choice of components may include, for textile care, the consideration of the type of textile to be cleaned, the type and/or degree of soiling, the temperature at which cleaning is to take place, and the formulation of the detergent product. Although components mentioned below are categorized by general header according to a particular functionality, this is not to be construed as a limitation, as a component may comprise additional functionalities as will be appreciated by the skilled artisan.

An ADW (Automatic Dish Wash) composition may comprise an enzyme of the present invention in combination with one or more additional ADW composition components. The choice of additional components is within the skill of the artisan and includes conventional ingredients, including the exemplary non-limiting components set forth below.

Surfactants

The detergent composition may comprise one or more surfactants, which may be anionic and/or cationic and/or non-ionic and/or semi-polar and/or zwitterionic, or a mixture thereof. In a particular embodiment, the detergent composition includes a mixture of one or more nonionic surfactants and one or more anionic surfactants. The surfactant(s) is typically present at a level of from about 0.1% to 60% by weight, such as about 1% to about 40%, or about 3% to about 20%, or about 3% to about 10%. The surfactant(s) is chosen based on the desired cleaning application, and may include any conventional surfactant(s) known in the art.

When included therein the detergent will usually contain from about 1% to about 40% by weight of an anionic surfactant, such as from about 5% to about 30%, including from about 5% to about 15%, or from about 15% to about 20%, or from about 20% to about 25% of an anionic surfactant. Non-limiting examples of anionic surfactants include sulfates and sulfonates, in particular, linear alkylbenzenesulfonates (LAS), isomers of LAS, branched alkylbenzenesulfonates (BABS), phenylalkanesulfonates, alpha-olefinsulfonates (AOS), olefin sulfonates, alkene sulfonates, alkane-2,3-diylbis(sulfates), hydroxyalkanesulfonates and disulfonates, alkyl sulfates (AS) such as sodium dodecyl sulfate (SDS), fatty alcohol sulfates (FAS), primary alcohol sulfates (PAS), alcohol ethersulfates (AES or AEOS or FES, also known as alcohol ethoxysulfates or fatty alcohol ether sulfates), secondary alkanesulfonates (SAS), paraffin sulfonates (PS), ester sulfonates, sulfonated fatty acid glycerol esters, alpha-sulfo fatty acid methyl esters (alpha-SFMe or SES) including methyl ester sulfonate (MES), alkyl- or alkenylsuccinic acid, dodecenyl/tetradecenyl succinic acid (DTSA), fatty acid derivatives of amino acids, diesters and monoesters of sulfo-succinic acid or salt of fatty acids (soap), and combinations thereof.

When included therein the detergent will usually contain from about 1% to about 40% by weigh of a cationic surfactant, for example from about 0.5% to about 30%, in particular from about 1% to about 20%, from about 3% to about 10%, such as from about 3% to about 5%, from about 8% to about 12% or from about 10% to about 12%. Non-limiting examples of cationic surfactants include alkyldimethylethanolamine quat (ADMEAQ), cetyltrimethylammonium bromide (CTAB), dimethyldistearylammonium chloride (DSDMAC), and alkylbenzyldimethylammonium, alkyl quaternary ammonium compounds, alkoxylated quaternary ammonium (AQA) compounds, ester quats, and combinations thereof.

When included therein the detergent will usually contain from about 0.2% to about 40% by weight of a nonionic surfactant, for example from about 0.5% to about 30%, in particular from about 1% to about 20%, from about 3% to about 10%, such as from about 3% to about 5%, from about 8% to about 12%, or from about 10% to about 12%. Non-limiting examples of nonionic surfactants include alcohol ethoxylates (AE or AEO), alcohol propoxylates, propoxylated fatty alcohols (PFA), alkoxylated fatty acid alkyl esters, such as ethoxylated and/or propoxylated fatty acid alkyl esters, alkylphenol ethoxylates (APE), nonylphenol ethoxylates (NPE), alkylpolyglycosides (APG), alkoxylated amines, fatty acid monoethanolamides (FAM), fatty acid diethanolamides (FADA), ethoxylated fatty acid monoethanolamides (EFAM), propoxylated fatty acid monoethanolamides (PFAM), polyhydroxyalkyl fatty acid amides, or N-acyl N-alkyl derivatives of glucosamine (glucamides, GA, or fatty acid glucamides, FAGA), as well as products available under the trade names SPAN and TWEEN, and combinations thereof.

When included therein the detergent will usually contain from about 0% to about 10% by weight of a semipolar surfactant. Non-limiting examples of semipolar surfactants include amine oxides (AO) such as alkyldimethylamineoxide, N-(coco alkyl)-N,N-dimethylamine oxide and N-(tallow-alkyl)-N,N-bis(2-hydroxyethyl)amine oxide, and combinations thereof.

When included therein the detergent will usually contain from about 0% to about 10% by weight of a zwitterionic surfactant. Non-limiting examples of zwitterionic surfactants include betaines such as alkyldimethylbetaines, sulfobetaines, and combinations thereof.

Hydrotropes

A hydrotrope is a compound that solubilises hydrophobic compounds in aqueous solutions (or oppositely, polar substances in a non-polar environment). Typically, hydrotropes have both hydrophilic and a hydrophobic character (so-called amphiphilic properties as known from surfactants); however the molecular structure of hydrotropes generally do not favor spontaneous self-aggregation, see e.g. review by Hodgdon and Kaler (2007), Current Opinion in Colloid & Interface Science 12: 121-128. Hydrotropes do not display a critical concentration above which self-aggregation occurs as found for surfactants and lipids forming miceller, lamellar or other well defined meso-phases. Instead, many hydrotropes show a continuous-type aggregation process where the sizes of aggregates grow as concentration increases. However, many hydrotropes alter the phase behavior, stability, and colloidal properties of systems containing substances of polar and non-polar character, including mixtures of water, oil, surfactants, and polymers. Hydrotropes are classically used across industries from pharma, personal care, food, to technical applications. Use of hydrotropes in detergent compositions allow for example more concentrated formulations of surfactants (as in the process of compacting liquid detergents by removing water) without inducing undesired phenomena such as phase separation or high viscosity.

The detergent may contain 0-10% by weight, for example 0-5% by weight, such as about 0.5 to about 5%, or about 3% to about 5%, of a hydrotrope. Any hydrotrope known in the art for use in detergents may be utilized. Non-limiting examples of hydrotropes include sodium benzenesulfonate, sodium p-toluene sulfonate (STS), sodium xylene sulfonate (SXS), sodium cumene sulfonate (SCS), sodium cymene sulfonate, amine oxides, alcohols and polyglycolethers, sodium hydroxynaphthoate, sodium hydroxynaphthalene sulfonate, sodium ethylhexyl sulfate, and combinations thereof.

Builders and Co-Builders

The detergent composition may contain about 0-65% by weight, such as about 5% to about 50% of a detergent builder or co-builder, or a mixture thereof. In a dish wash detergent, the level of builder is typically 40-65%, particularly 50-65%. The builder and/or co-builder may particularly be a chelating agent that forms water-soluble complexes with Ca and Mg. Any builder and/or co-builder known in the art for use in detergents may be utilized. Non-limiting examples of builders include zeolites, diphosphates (pyrophosphates), triphosphates such as sodium triphosphate (STP or STPP), carbonates such as sodium carbonate, soluble silicates such as sodium metasilicate, layered silicates (e.g., SKS-6 from Hoechst), ethanolamines such as 2-aminoethan-1-ol (MEA), diethanolamine (DEA, also known as 2,2′-iminodiethan-1-ol), triethanolamine (TEA, also known as 2,2′,2″-nitrilotriethan-1-ol), and (carboxymethyl)inulin (CMI), and combinations thereof.

The detergent composition may also contain 0-50% by weight, such as about 5% to about 30%, of a detergent co-builder. The detergent composition may include a co-builder alone, or in combination with a builder, for example a zeolite builder. Non-limiting examples of co-builders include homopolymers of polyacrylates or copolymers thereof, such as poly(acrylic acid) (PAA) or copoly(acrylic acid/maleic acid) (PAA/PMA). Further non-limiting examples include citrate, chelators such as aminocarboxylates, aminopolycarboxylates and phosphonates, and alkyl- or alkenylsuccinic acid. Additional specific examples include 2,2′,2″-nitrilotriacetic acid (NTA), ethylenediaminetetraacetic acid (EDTA), diethylenetriaminepentaacetic acid (DTPA), iminodisuccinic acid (IDS), ethylenediamine-N,N′-disuccinic acid (EDDS), methylglycinediacetic acid (MGDA), glutamic acid-N,N-diacetic acid (GLDA), 1-hydroxyethane-1,1-diphosphonic acid (HEDP), ethylenediaminetetra(methylenephosphonic acid) (EDTMPA), diethylenetriaminepentakis(methylenephosphonic acid) (DTMPA or DTPMPA), N-(2-hydroxyethyl)iminodiacetic acid (EDG), aspartic acid-N-monoacetic acid (ASMA), aspartic acid-N,N-diacetic acid (ASDA), aspartic acid-N-monopropionic acid (ASMP), iminodisuccinic acid (IDA), N-(2-sulfomethyl)-aspartic acid (SMAS), N-(2-sulfoethyl)-aspartic acid (SEAS), N-(2-sulfomethyl)-glutamic acid (SMGL), N-(2-sulfoethyl)-glutamic acid (SEGL), N-methyliminodiacetic acid (MIDA), alpha-alanine-N,N-diacetic acid (α-ALDA), serine-N,N-diacetic acid (SEDA), isoserine-N,N-diacetic acid (ISDA), phenylalanine-N,N-diacetic acid (PHDA), anthranilic acid-N,N-diacetic acid (ANDA), sulfanilic acid-N,N-diacetic acid (SLDA), taurine-N,N-diacetic acid (TUDA) and sulfomethyl-N,N-diacetic acid (SMDA), N-(2-hydroxyethyl)ethylenediamine-N,N′,N″-triacetic acid (HEDTA), diethanolglycine (DEG), diethylenetriamine penta(methylenephosphonic acid) (DTPMP), aminotris(methylenephosphonic acid) (ATMP), and combinations and salts thereof. Further exemplary builders and/or co-builders are described in, e.g., WO 09/102854, U.S. Pat. No. 5,977,053

Bleaching Systems

The detergent may contain 0-30% by weight, such as about 1% to about 20%, of a bleaching system. Any bleaching system known in the art for use in detergents may be utilized. Suitable bleaching system components include bleaching catalysts, photobleaches, bleach activators, sources of hydrogen peroxide such as sodium percarbonate, sodium perborates and hydrogen peroxide-urea (1:1), preformed peracids and mixtures thereof. Suitable preformed peracids include, but are not limited to, peroxycarboxylic acids and salts, diperoxydicarboxylic acids, perimidic acids and salts, peroxymonosulfuric acids and salts, for example, Oxone (R), and mixtures thereof. Non-limiting examples of bleaching systems include peroxide-based bleaching systems, which may comprise, for example, an inorganic salt, including alkali metal salts such as sodium salts of perborate (usually mono- or tetra-hydrate), percarbonate, persulfate, perphosphate, persilicate salts, in combination with a peracid-forming bleach activator. The term bleach activator is meant herein as a compound which reacts with hydrogen peroxide to form a peracid via perhydrolysis. The peracid thus formed constitutes the activated bleach. Suitable bleach activators to be used herein include those belonging to the class of esters, amides, imides or anhydrides. Suitable examples are tetraacetylethylenediamine (TAED), sodium 4-[(3,5,5-trimethylhexanoyl)oxy]benzene-1-sulfonate (ISONOBS), 4-(dodecanoyloxy)benzene-1-sulfonate (LOBS), 4-(decanoyloxy)benzene-1-sulfonate, 4-(decanoyloxy)benzoate (DOBS or DOBA), 4-(nonanoyloxy)benzene-1-sulfonate (NOBS), and/or those disclosed in WO98/17767. A particular family of bleach activators of interest was disclosed in EP624154 and particularly preferred in that family is acetyl triethyl citrate (ATC). ATC or a short chain triglyceride like triacetin has the advantage that it is environmentally friendly Furthermore acetyl triethyl citrate and triacetin have good hydrolytical stability in the product upon storage and are efficient bleach activators. Finally ATC is multifunctional, as the citrate released in the perhydrolysis reaction may function as a builder. Alternatively, the bleaching system may comprise peroxyacids of, for example, the amide, imide, or sulfone type. The bleaching system may also comprise peracids such as 6-(phthalimido)peroxyhexanoic acid (PAP). The bleaching system may also include a bleach catalyst. In some embodiments the bleach component may be an organic catalyst selected from the group consisting of organic catalysts having the following formulae:

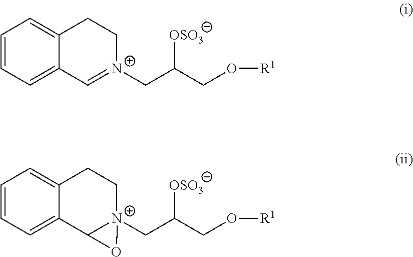

(iii) and mixtures thereof;

wherein each R¹ is independently a branched alkyl group containing from 9 to 24 carbons or linear alkyl group containing from 11 to 24 carbons, preferably each R¹ is independently a branched alkyl group containing from 9 to 18 carbons or linear alkyl group containing from 11 to 18 carbons, more preferably each R¹ is independently selected from the group consisting of 2-propylheptyl, 2-butyloctyl, 2-pentylnonyl, 2-hexyldecyl, dodecyl, tetradecyl, hexadecyl, octadecyl, isononyl, isodecyl, isotridecyl and isopentadecyl. Other exemplary bleaching systems are described, e.g. in WO2007/087258, WO2007/087244, WO2007/087259, EP1867708 (Vitamin K) and WO2007/087242. Suitable photobleaches may for example be sulfonated zinc or aluminium phthalocyanines.

Preferably the bleach component comprises a source of peracid in addition to bleach catalyst, particularly organic bleach catalyst. The source of peracid may be selected from (a) pre-formed peracid; (b) percarbonate, perborate or persulfate salt (hydrogen peroxide source) preferably in combination with a bleach activator; and (c) perhydrolase enzyme and an ester for forming peracid in situ in the presence of water in a textile or hard surface treatment step.

Polymers

The detergent may contain 0-10% by weight, such as 0.5-5%, 2-5%, 0.5-2% or 0.2-1% of a polymer. Any polymer known in the art for use in detergents may be utilized. The polymer may function as a co-builder as mentioned above, or may provide antiredeposition, fiber protection, soil release, dye transfer inhibition, grease cleaning and/or anti-foaming properties. Some polymers may have more than one of the above-mentioned properties and/or more than one of the below-mentioned motifs. Exemplary polymers include (carboxymethyl)cellulose (CMC), poly(vinyl alcohol) (PVA), poly(vinylpyrrolidone) (PVP), poly(ethyleneglycol) or poly(ethylene oxide) (PEG), ethoxylated poly(ethyleneimine), carboxymethyl inulin (CMI), and polycarboxylates such as PAA, PAA/PMA, poly-aspartic acid, and lauryl methacrylate/acrylic acid copolymers, hydrophobically modified CMC (HM-CMC) and silicones, copolymers of terephthalic acid and oligomeric glycols, copolymers of poly(ethylene terephthalate) and poly(oxyethene terephthalate) (PET-POET), PVP, poly(vinylimidazole) (PVI), poly(vinylpyridine-N-oxide) (PVPO or PVPNO) and polyvinylpyrrolidone-vinylimidazole (PVPVI). Further exemplary polymers include sulfonated polycarboxylates, polyethylene oxide and polypropylene oxide (PEO-PPO) and diquaternium ethoxy sulfate. Other exemplary polymers are disclosed in, e.g., WO 2006/130575. Salts of the above-mentioned polymers are also contemplated.

Fabric Hueing Agents

The detergent compositions may also include fabric hueing agents such as dyes or pigments, which when formulated in detergent compositions can deposit onto a fabric when said fabric is contacted with a wash liquor comprising said detergent compositions and thus altering the tint of said fabric through absorption/reflection of visible light. Fluorescent whitening agents emit at least some visible light. In contrast, fabric hueing agents alter the tint of a surface as they absorb at least a portion of the visible light spectrum. Suitable fabric hueing agents include dyes and dye-clay conjugates, and may also include pigments. Suitable dyes include small molecule dyes and polymeric dyes. Suitable small molecule dyes include small molecule dyes selected from the group consisting of dyes falling into the Colour Index (C.I.) classifications of Direct Blue, Direct Red, Direct Violet, Acid Blue, Acid Red, Acid Violet, Basic Blue, Basic Violet and Basic Red, or mixtures thereof, for example as described in WO2005/03274, WO2005/03275, WO2005/03276 and EP1876226 (hereby incorporated by reference). The detergent composition preferably comprises from about 0.00003 wt % to about 0.2 wt %, from about 0.00008 wt % to about 0.05 wt %, or even from about 0.0001 wt % to about 0.04 wt % fabric hueing agent. The composition may comprise from 0.0001 wt % to 0.2 wt % fabric hueing agent, this may be especially preferred when the composition is in the form of a unit dose pouch. Suitable hueing agents are also disclosed in, e.g. WO 2007/087257 and WO2007/087243.

Additional Enzymes

The detergent additive as well as the detergent composition may comprise one or more additional enzymes such as a protease, a lipase, a cutinase, an amylase, a carbohydrase, a cellulase, a pectinase, a mannanase, an arabinase, a galactanase, a xylanase, an oxidase, e.g., a laccase, and/or a peroxidase and/or a xanthan lyase.

In general the properties of the selected enzyme(s) should be compatible with the selected detergent, (i.e., pH-optimum, compatibility with other enzymatic and non-enzymatic ingredients, etc.), and the enzyme(s) should be present in effective amounts.

Cellulases

Suitable cellulases include those of bacterial or fungal origin. Chemically modified or protein engineered mutants are included. Suitable cellulases include cellulases from the genera Bacillus, Pseudomonas, Humicola, Fusarium, Thielavia, Acremonium, e.g., the fungal cellulases produced from Humicola insolens, Myceliophthora thermophila and Fusarium oxysporum disclosed in U.S. Pat. No. 4,435,307, U.S. Pat. No. 5,648,263, U.S. Pat. No. 5,691,178, U.S. Pat. No. 5,776,757 and WO 89/09259.

Especially suitable cellulases are the alkaline or neutral cellulases having colour care benefits. Examples of such cellulases are cellulases described in EP 0 495 257, EP 0 531 372, WO 96/11262, WO 96/29397, WO 98/08940. Other examples are cellulase variants such as those described in WO 94/07998, EP 0 531 315, U.S. Pat. No. 5,457,046, U.S. Pat. No. 5,686,593, U.S. Pat. No. 5,763,254, WO 95/24471, WO 98/12307 and WO99/001544.

Other cellulases are endo-beta-1,4-glucanase enzyme having a sequence of at least 97% identity to the amino acid sequence of position 1 to position 773 of SEQ ID NO:2 of WO 2002/099091 or a family 44 xyloglucanase, which a xyloglucanase enzyme having a sequence of at least 60% identity to positions 40-559 of SEQ ID NO: 2 of WO 2001/062903.

Commercially available cellulases include Celluzyme™, and Carezyme™ (Novozymes A/S) Carezyme Premium™ (Novozymes A/S), Celluclean™ (Novozymes A/S), Celluclean Classic™ (Novozymes A/S), Cellusoft™ (Novozymes A/S), Whitezyme™ (Novozymes A/S), Clazinase™, and Puradax HA™ (Genencor International Inc.), and KAC-500(B)™ (Kao Corporation).

Mannanases

Suitable mannanases include those of bacterial or fungal origin. Chemically or genetically modified mutants are included. The mannanase may be an alkaline mannanase of Family 5 or 26. It may be a wild-type from Bacillus or Humicola, particularly B. agaradhaerens, B. licheniformis, B. halodurans, B. clausii, or H. insolens. Suitable mannanases are described in WO 1999/064619. A commercially available mannanase is Mannaway (Novozymes A/S).

Xanthan Lyases

Suitable xanthan lyases include those of plant, bacterial or fungal origin. Chemically modified or protein engineered mutants are included. Examples of useful enzymes include the xanthan lyases disclosed in WO2013167581 and shown herein as SEQ ID NO:21, 22, 23 and 24.

Proteases

Suitable proteases include those of bacterial, fungal, plant, viral or animal origin e.g. vegetable or microbial origin. Microbial origin is preferred. Chemically modified or protein engineered mutants are included. It may be an alkaline protease, such as a serine protease or a metalloprotease. A serine protease may for example be of the S1 family, such as trypsin, or the S8 family such as subtilisin. A metalloproteases protease may for example be a thermolysin from e.g. family M4 or other metalloprotease such as those from M5, M7 or M8 families.

The term “subtilases” refers to a sub-group of serine protease according to Siezen et al., Protein Engng. 4 (1991) 719-737 and Siezen et al. Protein Science 6 (1997) 501-523. Serine proteases are a subgroup of proteases characterized by having a serine in the active site, which forms a covalent adduct with the substrate. The subtilases may be divided into 6 sub-divisions, i.e. the Subtilisin family, the Thermitase family, the Proteinase K family, the Lantibiotic peptidase family, the Kexin family and the Pyrolysin family.

Examples of subtilases are those derived from Bacillus such as Bacillus lentus, B. alkalophilus, B. subtilis, B. amyloliquefaciens, Bacillus pumilus and Bacillus gibsonii described in; U.S. Pat. No. 7,262,042 and WO09/021867, and subtilisin lentus, subtilisin Novo, subtilisin Carlsberg, Bacillus licheniformis, subtilisin BPN′, subtilisin 309, subtilisin 147 and subtilisin 168 described in WO89/06279 and protease PD138 described in (WO93/18140). Other useful proteases may be those described in WO92/175177, WO01/016285, WO02/026024 and WO02/016547. Examples of trypsin-like proteases are trypsin (e.g. of porcine or bovine origin) and the Fusarium protease described in WO89/06270, WO94/25583 and WO05/040372, and the chymotrypsin proteases derived from Cellumonas described in WO05/052161 and WO05/052146.

A further preferred protease is the alkaline protease from Bacillus lentus DSM 5483, as described for example in WO95/23221, and variants thereof which are described in WO92/21760, WO95/23221, EP1921147 and EP1921148.

Examples of metalloproteases are the neutral metalloprotease as described in WO07/044993 (Genencor Int.) such as those derived from Bacillus amyloliquefaciens.

Examples of useful proteases are the variants described in: WO92/19729, WO96/034946, WO98/20115, WO98/20116, WO99/011768, WO01/44452, WO03/006602, WO04/03186, WO04/041979, WO07/006305, WO11/036263, WO11/036264, especially the variants with substitutions in one or more of the following positions: 3, 4, 9, 15, 27, 36, 57, 68, 76, 87, 95, 96, 97, 98, 99, 100, 101, 102, 103, 104, 106, 118, 120, 123, 128, 129, 130, 160, 167, 170, 194, 195, 199, 205, 206, 217, 218, 222, 224, 232, 235, 236, 245, 248, 252 and 274 using the BPN′ numbering. More preferred the subtilase variants may comprise the mutations: S3T, V41, S9R, A15T, K27R, *36D, V68A, N76D, N87S,R, *97E, A98S, S99G,D,A, S99AD, S101G,M,R S103A, V1041,Y,N, S106A, G118V,R, H120D,N, N123S, S128L, P129Q, S130A, G160D, Y167A, R170S, A194P, G195E, V199M, V2051, L217D, N218D, M222S, A232V, K235L, Q236H, Q245R, N252K, T274A (using BPN′ numbering).

Suitable commercially available protease enzymes include those sold under the trade names Alcalase®, Duralase™, Durazym™, Relase®, Relase® Ultra, Savinase®, Savinase® Ultra, Primase®, Polarzyme®, Kannase®, Liquanase®, Liquanase® Ultra, Ovozyme®, Coronase®, Coronase® Ultra, Blaze®, Neutrase®, Everlase® and Esperase® (Novozymes A/S), those sold under the tradename Maxatase®, Maxacal®, Maxapem®, Purafect®, Purafect Prime®, Purafect MA®, Purafect Ox®, Purafect OxP®, Puramax®, Properase®, FN2®, FN3®, FN4®, Excellase®, Eraser®, Opticlean® and Optimase® (Danisco/DuPont), Axapem™ (Gist-Brocases N.V.), BLAP (sequence shown in FIG. 29 of U.S. Pat. No. 5,352,604) and variants hereof (Henkel AG) and KAP (Bacillus alkalophilus subtilisin) from Kao.

Lipases and Cutinases

Suitable lipases and cutinases include those of bacterial or fungal origin. Chemically modified or protein engineered mutant enzymes are included. Examples include lipase from Thermomyces, e.g. from T. lanuginosus (previously named Humicola lanuginosa) as described in EP258068 and EP305216, cutinase from Humicola, e.g. H. insolens (WO96/13580), lipase from strains of Pseudomonas (some of these now renamed to Burkholderia), e.g. P. alcaligenes or P. pseudoalcaligenes (EP218272), P. cepacia (EP331376), P. sp. strain SD705 (WO95/06720 & WO96/27002), P. wisconsinensis (WO96/12012), GDSL-type Streptomyces lipases (WO10/065455), cutinase from Magnaporthe grisea (WO10/107560), cutinase from Pseudomonas mendocina (U.S. Pat. No. 5,389,536), lipase from Thermobifida fusca (WO11/084412), Geobacillus stearothermophilus lipase (WO11/084417), lipase from Bacillus subtilis (WO11/084599), and lipase from Streptomyces griseus (WO11/150157) and S. pristinaespiralis (WO12/137147).

Other examples are lipase variants such as those described in EP407225, WO92/05249, WO94/01541, WO94/25578, WO95/14783, WO95/30744, WO95/35381, WO95/22615, WO96/00292, WO97/04079, WO97/07202, WO00/34450, WO00/60063, WO01/92502, WO07/87508 and WO09/109500.

Preferred commercial lipase products include Lipolase™, Lipex™; Lipolex™ and Lipoclean™ (Novozymes A/S), Lumafast (originally from Genencor) and Lipomax (originally from Gist-Brocades).

Still other examples are lipases sometimes referred to as acyltransferases or perhydrolases, e.g. acyltransferases with homology to Candida antarctica lipase A (WO 10/111143), acyltransferase from Mycobacterium smegmatis (WO05/56782), perhydrolases from the CE 7 family (WO09/67279), and variants of the M. smegmatis perhydrolase in particular the S54V variant used in the commercial product Gentle Power Bleach from Huntsman Textile Effects Pte Ltd (WO10/100028).

Amylases

Suitable amylases which can be used together with the enzyme of the invention may be an alpha-amylase or a glucoamylase and may be of bacterial or fungal origin. Chemically modified or protein engineered mutants are included. Amylases include, for example, alpha-amylases obtained from Bacillus, e.g., a special strain of Bacillus licheniformis, described in more detail in GB 1,296,839.

Suitable amylases include amylases having SEQ ID NO: 2 in WO 95/10603 or variants having 90% sequence identity to SEQ ID NO: 3 thereof. Preferred variants are described in WO 94/02597, WO 94/18314, WO 97/43424 and SEQ ID NO: 4 of WO 99/019467, such as variants with substitutions in one or more of the following positions: 15, 23, 105, 106, 124, 128, 133, 154, 156, 178, 179, 181, 188, 190, 197, 201, 202, 207, 208, 209, 211, 243, 264, 304, 305, 391,408, and 444.

Different suitable amylases include amylases having SEQ ID NO: 6 in WO 02/010355 or variants thereof having 90% sequence identity to SEQ ID NO: 6. Preferred variants of SEQ ID NO: 6 are those having a deletion in positions 181 and 182 and a substitution in position 193.

Other amylases which are suitable are hybrid alpha-amylase comprising residues 1-33 of the alpha-amylase derived from B. amyloliquefaciens shown in SEQ ID NO: 6 of WO 2006/066594 and residues 36-483 of the B. licheniformis alpha-amylase shown in SEQ ID NO: 4 of WO 2006/066594 or variants having 90% sequence identity thereof. Preferred variants of this hybrid alpha-amylase are those having a substitution, a deletion or an insertion in one of more of the following positions: G48, T49, G107, H156, A181, N190, M197, I201, A209 and Q264. Most preferred variants of the hybrid alpha-amylase comprising residues 1-33 of the alpha-amylase derived from B. amyloliquefaciens shown in SEQ ID NO: 6 of WO 2006/066594 and residues 36-483 of SEQ ID NO: 4 are those having the substitutions:

M197T;

H156Y+A181T+N190F+A209V+Q264S; or

G48A+T49I+G107A+H156Y+A181T+N190F+I201F+A209V+Q264S.

Further amylases which are suitable are amylases having SEQ ID NO: 6 in WO 99/019467 or variants thereof having 90% sequence identity to SEQ ID NO: 6. Preferred variants of SEQ ID NO: 6 are those having a substitution, a deletion or an insertion in one or more of the following positions: R181, G182, H183, G184, N195, I206, E212, E216 and K269. Particularly preferred amylases are those having deletion in positions R181 and G182, or positions H183 and G184.

Additional amylases which can be used are those having SEQ ID NO: 1, SEQ ID NO: 3, SEQ ID NO: 2 or SEQ ID NO: 7 of WO 96/023873 or variants thereof having 90% sequence identity to SEQ ID NO: 1, SEQ ID NO: 2, SEQ ID NO: 3 or SEQ ID NO: 7. Preferred variants of SEQ ID NO: 1, SEQ ID NO: 2, SEQ ID NO: 3 or SEQ ID NO: 7 are those having a substitution, a deletion or an insertion in one or more of the following positions: 140, 181, 182, 183, 184, 195, 206, 212, 243, 260, 269, 304 and 476, using SEQ ID 2 of WO 96/023873 for numbering. More preferred variants are those having a deletion in two positions selected from 181, 182, 183 and 184, such as 181 and 182, 182 and 183, or positions 183 and 184. Most preferred amylase variants of SEQ ID NO: 1, SEQ ID NO: 2 or SEQ ID NO: 7 are those having a deletion in positions 183 and 184 and a substitution in one or more of positions 140, 195, 206, 243, 260, 304 and 476.

Other amylases which can be used are amylases having SEQ ID NO: 2 of WO 08/153815, SEQ ID NO: 10 in WO 01/66712 or variants thereof having 90% sequence identity to SEQ ID NO: 2 of WO 08/153815 or 90% sequence identity to SEQ ID NO: 10 in WO 01/66712. Preferred variants of SEQ ID NO: 10 in WO 01/66712 are those having a substitution, a deletion or an insertion in one of more of the following positions: 176, 177, 178, 179, 190, 201, 207, 211 and 264.

Further suitable amylases are amylases having SEQ ID NO: 2 of WO 09/061380 or variants having 90% sequence identity to SEQ ID NO: 2 thereof. Preferred variants of SEQ ID NO: 2 are those having a truncation of the C-terminus and/or a substitution, a deletion or an insertion in one of more of the following positions: Q87, Q98, S125, N128, T131, T165, K178, R180, S181, T182, G183, M201, F202, N225, S243, N272, N282, Y305, R309, D319, Q320, Q359, K444 and G475. More preferred variants of SEQ ID NO: 2 are those having the substitution in one of more of the following positions: Q87E,R, Q98R, S125A, N128C, T131I, T165I, K178L, T182G, M201L, F202Y, N225E,R, N272E,R, S243Q,A,E,D, Y305R, R309A, Q320R, Q359E, K444E and G475K and/or deletion in position R180 and/or S181 or of T182 and/or G183. Most preferred amylase variants of SEQ ID NO: 2 are those having the substitutions:

N128C+K178L+T182G+Y305R+G475K;

N128C+K178L+T182G+F202Y+Y305R+D319T+G475K;

S125A+N128C+K178L+T182G+Y305R+G475K; or

S125A+N128C+T131I+T165I+K178L+T182G+Y305R+G475K wherein the variants are C-terminally truncated and optionally further comprises a substitution at position 243 and/or a deletion at position 180 and/or position 181.

Further suitable amylases are amylases having SEQ ID NO: 1 of WO13184577 or variants having 90% sequence identity to SEQ ID NO: 1 thereof. Preferred variants of SEQ ID NO: 1 are those having a substitution, a deletion or an insertion in one of more of the following positions: K176, R178, G179, T180, G181, E187, N192, M199, I203, S241, R458, T459, D460, G476 and G477. More preferred variants of SEQ ID NO: 1 are those having the substitution in one of more of the following positions: K176L, E187P, N192FYH, M199L, I203YF, S241QADN, R458N, T459S, D460T, G476K and G477K and/or deletion in position R178 and/or S179 or of T180 and/or G181. Most preferred amylase variants of SEQ ID NO: 1 are those having the substitutions:

E187P+I203Y+G476K

E187P+I203Y+R458N+T459S+D460T+G476K

wherein the variants optionally further comprises a substitution at position 241 and/or a deletion at position 178 and/or position 179.

Further suitable amylases are amylases having SEQ ID NO: 1 of WO10104675 or variants having 90% sequence identity to SEQ ID NO: 1 thereof. Preferred variants of SEQ ID NO: 1 are those having a substitution, a deletion or an insertion in one of more of the following positions: N21, D97, V128 K177, R179, S180, 1181, G182, M200, L204, E242, G477 and G478. More preferred variants of SEQ ID NO: 1 are those having the substitution in one of more of the following positions: N21D, D97N, V128I K177L, M200L, L204YF, E242QA, G477K and G478K and/or deletion in position R179 and/or S180 or of 1181 and/or G182. Most preferred amylase variants of SEQ ID NO: 1 are those having the substitutions:

N21D+D97N+V128I

wherein the variants optionally further comprises a substitution at position 200 and/or a deletion at position 180 and/or position 181.

Other suitable amylases are the alpha-amylase having SEQ ID NO: 12 in WO01/66712 or a variant having at least 90% sequence identity to SEQ ID NO: 12. Preferred amylase variants are those having a substitution, a deletion or an insertion in one of more of the following positions of SEQ ID NO: 12 in WO01/66712: R28, R118, N174; R181, G182, D183, G184, G186, W189, N195, M202, Y298, N299, K302, S303, N306, R310, N314; R320, H324, E345, Y396, R400, W439, R444, N445, K446, Q449, R458, N471, N484. Particular preferred amylases include variants having a deletion of D183 and G184 and having the substitutions R118K, N195F, R320K and R458K, and a variant additionally having substitutions in one or more position selected from the group: M9, G149, G182, G186, M202, T257, Y295, N299, M323, E345 and A339, most preferred a variant that additionally has substitutions in all these positions.

Other examples are amylase variants such as those described in WO2011/098531, WO2013/001078 and WO2013/001087.

Commercially available amylases are Duramyl™, Termamyl™, Fungamyl™, Stainzyme™, Stainzyme Plus™, Natalase™, Liquozyme X and BAN™ (from Novozymes A/S), and Rapidase™, Purastar™/Effectenz™, Powerase, Preferenz S1000, Preferenz S100 and Preferenz S110 (from Genencor International Inc./DuPont).

Peroxidases/Oxidases

A peroxidase according to the invention is a peroxidase enzyme comprised by the enzyme classification EC 1.11.1.7, as set out by the Nomenclature Committee of the International Union of Biochemistry and Molecular Biology (IUBMB), or any fragment derived therefrom, exhibiting peroxidase activity.

Suitable peroxidases include those of plant, bacterial or fungal origin. Chemically modified or protein engineered mutants are included. Examples of useful peroxidases include peroxidases from Coprinopsis, e.g., from C. cinerea (EP 179,486), and variants thereof as those described in WO 93/24618, WO 95/10602, and WO 98/15257.

A peroxidase according to the invention also include a haloperoxidase enzyme, such as chloroperoxidase, bromoperoxidase and compounds exhibiting chloroperoxidase or bromoperoxidase activity. Haloperoxidases are classified according to their specificity for halide ions. Chloroperoxidases (E.C. 1.11.1.10) catalyze formation of hypochlorite from chloride ions.

In an embodiment, the haloperoxidase is a chloroperoxidase. Preferably, the haloperoxidase is a vanadium haloperoxidase, i.e., a vanadate-containing haloperoxidase. In a preferred method of the present invention the vanadate-containing haloperoxidase is combined with a source of chloride ion.

Haloperoxidases have been isolated from many different fungi, in particular from the fungus group dematiaceous hyphomycetes, such as Caldariomyces, e.g., C. fumago, Alternaria, Curvularia, e.g., C. verruculosa and C. inaequalis, Drechslera, Ulocladium and Botrytis.

Haloperoxidases have also been isolated from bacteria such as Pseudomonas, e.g., P. pyrrocinia and Streptomyces, e.g., S. aureofaciens.

In an preferred embodiment, the haloperoxidase is derivable from Curvularia sp., in particular Curvularia verruculosa or Curvularia inaequalis, such as C. inaequalis CBS 102.42 as described in WO 95/27046; or C. verruculosa CBS 147.63 or C. verruculosa CBS 444.70 as described in WO 97/04102; or from Drechslera hartlebii as described in WO 01/79459, Dendryphiella salina as described in WO 01/79458, Phaeotrichoconis crotalarie as described in WO 01/79461, or Geniculosporium sp. as described in WO 01/79460.

An oxidase according to the invention include, in particular, any laccase enzyme comprised by the enzyme classification EC 1.10.3.2, or any fragment derived therefrom exhibiting laccase activity, or a compound exhibiting a similar activity, such as a catechol oxidase (EC 1.10.3.1), an o-aminophenol oxidase (EC 1.10.3.4), or a bilirubin oxidase (EC 1.3.3.5).

Preferred laccase enzymes are enzymes of microbial origin. The enzymes may be derived from plants, bacteria or fungi (including filamentous fungi and yeasts).

Suitable examples from fungi include a laccase derivable from a strain of Aspergillus, Neurospora, e.g., N. crassa, Podospora, Botrytis, Collybia, Fomes, Lentinus, Pleurotus, Trametes, e.g., T. villosa and T. versicolor, Rhizoctonia, e.g., R. solani, Coprinopsis, e.g., C. cinerea, C. comatus, C. friesii, and C. plicatilis, Psathyrella, e.g., P. condelleana, Panaeolus, e.g., P. papilionaceus, Myceliophthora, e.g., M. thermophila, Schytalidium, e.g., S. thermophilum, Polyporus, e.g., P. pinsitus, Phlebia, e.g., P. radiata (WO 92/01046), or Coriolus, e.g., C. hirsutus (JP 2238885).

Suitable examples from bacteria include a laccase derivable from a strain of Bacillus.

A laccase derived from Coprinopsis or Myceliophthora is preferred; in particular a laccase derived from Coprinopsis cinerea, as disclosed in WO 97/08325; or from Myceliophthora thermophila, as disclosed in WO 95/33836.

The detergent enzyme(s) may be included in a detergent composition by adding separate additives containing one or more enzymes, or by adding a combined additive comprising all of these enzymes. A detergent additive, i.e., a separate additive or a combined additive, can be formulated, for example, as a granulate, liquid, slurry, etc. Preferred detergent additive formulations are granulates, in particular non-dusting granulates, liquids, in particular stabilized liquids, or slurries.

Non-dusting granulates may be produced, e.g. as disclosed in U.S. Pat. Nos. 4,106,991 and 4,661,452 and may optionally be coated by methods known in the art. Examples of waxy coating materials are polyethyleneglycol (PEG) with mean molar weights of 1000 to 20000; ethoxylated nonylphenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Examples of film-forming coating materials suitable for application by fluid bed techniques are given in GB 1483591. Liquid enzyme preparations may, for instance, be stabilized by adding a polyol such as propylene glycol, a sugar or sugar alcohol, lactic acid or boric acid according to established methods. Protected enzymes may be prepared according to the method disclosed in EP 238,216.

### Adjunct Materials

Any detergent components known in the art for use in detergents may also be utilized. Other optional detergent components include anti-corrosion agents, anti-shrink agents, anti-soil redeposition agents, anti-wrinkling agents, bactericides, binders, corrosion inhibitors, disintegrants/disintegration agents, dyes, enzyme stabilizers (including boric acid, borates, CMC, and/or polyols such as propylene glycol), fabric conditioners including clays, fillers/processing aids, fluorescent whitening agents/optical brighteners, foam boosters, foam (suds) regulators, perfumes, soil-suspending agents, softeners, suds suppressors, tarnish inhibitors, and wicking agents, either alone or in combination. Any ingredient known in the art for use in detergents may be utilized. The choice of such ingredients is well within the skill of the artisan.

Dispersants

The detergent compositions can also contain dispersants. In particular powdered detergents may comprise dispersants. Suitable water-soluble organic materials include the homo- or co-polymeric acids or their salts, in which the polycarboxylic acid comprises at least two carboxyl radicals separated from each other by not more than two carbon atoms. Suitable dispersants are for example described in Powdered Detergents, Surfactant science series volume 71, Marcel Dekker, Inc.

Dye Transfer Inhibiting Agents

The detergent compositions may also include one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. When present in a subject composition, the dye transfer inhibiting agents may be present at levels from about 0.0001% to about 10%, from about 0.01% to about 5% or even from about 0.1% to about 3% by weight of the composition.

Fluorescent Whitening Agent

The detergent compositions will preferably also contain additional components that may tint articles being cleaned, such as fluorescent whitening agent or optical brighteners.

Where present the brightener is preferably at a level of about 0.01% to about 0.5%. Any fluorescent whitening agent suitable for use in a laundry detergent composition may be used in the composition. The most commonly used fluorescent whitening agents are those belonging to the classes of diaminostilbene-sulfonic acid derivatives, diarylpyrazoline derivatives and bisphenyl-distyryl derivatives. Examples of the diaminostilbene-sulfonic acid derivative type of fluorescent whitening agents include the sodium salts of: 4,4′-bis-(2-diethanolamino-4-anilino-s-triazin-6-ylamino) stilbene-2,2′-disulfonate, 4,4′-bis-(2,4-dianilino-s-triazin-6-ylamino) stilbene-2.2′-disulfonate, 4,4′-bis-(2-anilino-4-(N-methyl-N-2-hydroxy-ethylamino)-s-triazin-6-ylamino) stilbene-2,2′-disulfonate, 4,4′-bis-(4-phenyl-1,2,3-triazol-2-yl)stilbene-2,2′-disulfonate and sodium 5-(2H-naphtho[1,2-d][1,2,3]triazol-2-yl)-2-[(E)-2-phenylvinyl]benzenesulfonate. Preferred fluorescent whitening agents are Tinopal DMS and Tinopal CBS available from Ciba-Geigy AG, Basel, Switzerland. Tinopal DMS is the disodium salt of 4,4′-bis-(2-morpholino-4-anilino-s-triazin-6-ylamino) stilbene-2,2′-disulfonate. Tinopal CBS is the disodium salt of 2,2′-bis-(phenyl-styryl)-disulfonate. Also preferred are fluorescent whitening agents is the commercially available Parawhite KX, supplied by Paramount Minerals and Chemicals, Mumbai, India. Other fluorescers suitable for use in the invention include the 1-3-diaryl pyrazolines and the 7-alkylaminocoumarins.

Suitable fluorescent brightener levels include lower levels of from about 0.01, from 0.05, from about 0.1 or even from about 0.2 wt % to upper levels of 0.5 or even 0.75 wt %.

Soil Release Polymers

The detergent compositions may also include one or more soil release polymers which aid the removal of soils from fabrics such as cotton and polyester based fabrics, in particular the removal of hydrophobic soils from polyester based fabrics. The soil release polymers may for example be nonionic or anionic terephthalte based polymers, polyvinyl caprolactam and related copolymers, vinyl graft copolymers, polyester polyamides see for example Chapter 7 in Powdered Detergents, Surfactant science series volume 71, Marcel Dekker, Inc. Another type of soil release polymers are amphiphilic alkoxylated grease cleaning polymers comprising a core structure and a plurality of alkoxylate groups attached to that core structure. The core structure may comprise a polyalkylenimine structure or a polyalkanolamine structure as described in detail in WO 2009/087523 (hereby incorporated by reference). Furthermore random graft co-polymers are suitable soil release polymers. Suitable graft co-polymers are described in more detail in WO 2007/138054, WO 2006/108856 and WO 2006/113314 (hereby incorporated by reference). Other soil release polymers are substituted polysaccharide structures especially substituted cellulosic structures such as modified cellulose deriviatives such as those described in EP 1867808 or WO 2003/040279 (both are hereby incorporated by reference). Suitable cellulosic polymers include cellulose, cellulose ethers, cellulose esters, cellulose amides and mixtures thereof. Suitable cellulosic polymers include anionically modified cellulose, nonionically modified cellulose, cationically modified cellulose, zwitterionically modified cellulose, and mixtures thereof. Suitable cellulosic polymers include methyl cellulose, carboxy methyl cellulose, ethyl cellulose, hydroxyl ethyl cellulose, hydroxyl propyl methyl cellulose, ester carboxy methyl cellulose, and mixtures thereof.

Anti-Redeposition Agents

The detergent compositions may also include one or more anti-redeposition agents such as carboxymethylcellulose (CMC), polyvinyl alcohol (PVA), polyvinylpyrrolidone (PVP), polyoxyethylene and/or polyethyleneglycol (PEG), homopolymers of acrylic acid, copolymers of acrylic acid and maleic acid, and ethoxylated polyethyleneimines. The cellulose based polymers described under soil release polymers above may also function as anti-redeposition agents.

Rheology Modifiers

The detergent compositions may also include one or more rheology modifiers, structurants or thickeners, as distinct from viscosity reducing agents. The rheology modifiers are selected from the group consisting of non-polymeric crystalline, hydroxy-functional materials, polymeric rheology modifiers which impart shear thinning characteristics to the aqueous liquid matrix of a liquid detergent composition. The rheology and viscosity of the detergent can be modified and adjusted by methods known in the art, for example as shown in EP 2169040.

### Formulation of Detergent Products

The detergent composition may be in any convenient form, e.g., a bar, a homogenous tablet, a tablet having two or more layers, a pouch having one or more compartments, a regular or compact powder, a granule, a paste, a gel, or a regular, compact or concentrated liquid.

Pouches can be configured as single or multicompartments. It can be of any form, shape and material which is suitable for hold the composition, e.g. without allowing the release of the composition to release of the composition from the pouch prior to water contact. The pouch is made from water soluble film which encloses an inner volume. Said inner volume can be divided into compartments of the pouch. Preferred films are polymeric materials preferably polymers which are formed into a film or sheet. Preferred polymers, copolymers or derivates thereof are selected polyacrylates, and water soluble acrylate copolymers, methyl cellulose, carboxy methyl cellulose, sodium dextrin, ethyl cellulose, hydroxyethyl cellulose, hydroxypropyl methyl cellulose, malto dextrin, poly methacrylates, most preferably polyvinyl alcohol copolymers and, hydroxypropyl methyl cellulose (HPMC). Preferably the level of polymer in the film for example PVA is at least about 60%. Preferred average molecular weight will typically be about 20,000 to about 150,000. Films can also be of blended compositions comprising hydrolytically degradable and water soluble polymer blends such as polylactide and polyvinyl alcohol (known under the Trade reference M8630 as sold by MonoSol LLC, Indiana, USA) plus plasticisers like glycerol, ethylene glycerol, propylene glycol, sorbitol and mixtures thereof. The pouches can comprise a solid laundry cleaning composition or part components and/or a liquid cleaning composition or part components separated by the water soluble film. The compartment for liquid components can be different in composition than compartments containing solids: US2009/0011970 A1.

Detergent ingredients can be separated physically from each other by compartments in water dissolvable pouches or in different layers of tablets. Thereby negative storage interaction between components can be avoided. Different dissolution profiles of each of the compartments can also give rise to delayed dissolution of selected components in the wash solution.

A liquid or gel detergent, which is not unit dosed, may be aqueous, typically containing at least 20% by weight and up to 95% water, such as up to about 70% water, up to about 65% water, up to about 55% water, up to about 45% water, up to about 35% water. Other types of liquids, including without limitation, alkanols, amines, diols, ethers and polyols may be included in an aqueous liquid or gel. An aqueous liquid or gel detergent may contain from 0-30% organic solvent.

A liquid or gel detergent may be non-aqueous.

### Laundry Soap Bars

The enzymes of the invention may be added to laundry soap bars and used for hand washing laundry, fabrics and/or textiles. The term laundry soap bar includes laundry bars, soap bars, combo bars, syndet bars and detergent bars. The types of bar usually differ in the type of surfactant they contain, and the term laundry soap bar includes those containing soaps from fatty acids and/or synthetic soaps. The laundry soap bar has a physical form which is solid and not a liquid, gel or a powder at room temperature. The term solid is defined as a physical form which does not significantly change over time, i.e. if a solid object (e.g. laundry soap bar) is placed inside a container, the solid object does not change to fill the container it is placed in. The bar is a solid typically in bar form but can be in other solid shapes such as round or oval.

The laundry soap bar may contain one or more additional enzymes, protease inhibitors such as peptide aldehydes (or hydrosulfite adduct or hemiacetal adduct), boric acid, borate, borax and/or phenylboronic acid derivatives such as 4-formylphenylboronic acid, one or more soaps or synthetic surfactants, polyols such as glycerine, pH controlling compounds such as fatty acids, citric acid, acetic acid and/or formic acid, and/or a salt of a monovalent cation and an organic anion wherein the monovalent cation may be for example Na⁺, K⁺ or NH₄⁺ and the organic anion may be for example formate, acetate, citrate or lactate such that the salt of a monovalent cation and an organic anion may be, for example, sodium formate.

The laundry soap bar may also contain complexing agents like EDTA and HEDP, perfumes and/or different type of fillers, surfactants e.g. anionic synthetic surfactants, builders, polymeric soil release agents, detergent chelators, stabilizing agents, fillers, dyes, colorants, dye transfer inhibitors, alkoxylated polycarbonates, suds suppressers, structurants, binders, leaching agents, bleaching activators, clay soil removal agents, anti-redeposition agents, polymeric dispersing agents, brighteners, fabric softeners, perfumes and/or other compounds known in the art.

The laundry soap bar may be processed in conventional laundry soap bar making equipment such as but not limited to: mixers, plodders, e.g a two stage vacuum plodder, extruders, cutters, logo-stampers, cooling tunnels and wrappers. The invention is not limited to preparing the laundry soap bars by any single method. The premix may be added to the soap at different stages of the process. For example, the premix containing a soap, the enzyme of the invention, optionally one or more additional enzymes, a protease inhibitor, and a salt of a monovalent cation and an organic anion may be prepared and the mixture is then plodded. The enzyme of the invention and optional additional enzymes may be added at the same time as the protease inhibitor for example in liquid form. Besides the mixing step and the plodding step, the process may further comprise the steps of milling, extruding, cutting, stamping, cooling and/or wrapping.

### Formulation of Enzyme in Co-Granule

The enzyme of the invention may be formulated as a granule for example as a co-granule that combines one or more enzymes. Each enzyme will then be present in more granules securing a more uniform distribution of enzymes in the detergent. This also reduces the physical segregation of different enzymes due to different particle sizes. Methods for producing multi-enzyme co-granulates for the detergent industry is disclosed in the IP.com disclosure IPCOM000200739D.

Another example of formulation of enzymes by the use of co-granulates are disclosed in WO 2013/188331, which relates to a detergent composition comprising (a) a multi-enzyme co-granule; (b) less than 10 wt zeolite (anhydrous basis); and (c) less than 10 wt phosphate salt (anhydrous basis), wherein said enzyme co-granule comprises from 10 to 98 wt % moisture sink component and the composition additionally comprises from 20 to 80 wt % detergent moisture sink component.

WO 2013/188331 also relates to a method of treating and/or cleaning a surface, preferably a fabric surface comprising the steps of (i) contacting said surface with the detergent composition as claimed and described herein in aqueous wash liquor, (ii) rinsing and/or drying the surface.

The multi-enzyme co-granule may comprise an enzyme of the invention and (a) one or more enzymes selected from the group consisting of first-wash lipases, cleaning cellulases, xyloglucanases, perhydrolases, peroxidases, lipoxygenases, laccases and mixtures thereof; and (b) one or more enzymes selected from the group consisting of hemicellulases, proteases, care cellulases, cellobiose dehydrogenases, xylanases, phospholipases, esterases, cutinases, pectinases, mannanases, pectate lyases, keratinases, reductases, oxidases, phenoloxidases, ligninases, pullulanases, tannases, pentosanases, lichenases glucanases, arabinosidases, hyaluronidase, chondroitinase, amylases, and mixtures thereof.

### Use in Degrading Xanthan Gum

Xanthan gum is use as an ingredient in many consumer products including foods and cosmetics as well as in the oil and drilling industry. Therefore enzymes having xanthan degrading activity can be applied in improved cleaning processes, such as the easier removal of stains containing xanthan gum, as well as the degradation of xanthan gum which is often used in the oil and drilling industry. Thus the present invention is directed to the use of enzymes of the invention or compositions thereof to degrade xanthan gum. The present invention is also directed to the use of compositions comprising an enzyme of the invention and a xanthan lyase to degrade xanthan gum.

Degradation of xanthan gum may be measured using the viscosity reduction assay as described herein on xanthan gum. Xanthan degrading activity may alternatively be measured as reducing ends on xanthan gum using the colorimetric assay developed by Lever (1972), Anal. Biochem. 47: 273-279, 1972.

Use in Detergents

The enzymes of the invention or compositions thereof may be used in cleaning processes such as the laundering of textiles and fabrics (e.g. household laundry washing and industrial laundry washing), as well as household and industrial hard surface cleaning, such as dish wash. The enzymes of the invention may be added to a detergent composition comprising of one or more detergent components.

An embodiment is the use of enzymes of the invention together with xanthan lyases or compositions thereof in cleaning processes such as the laundering of textiles and fabrics (e.g. household laundry washing and industrial laundry washing), as well as household and industrial hard surface cleaning, such as dish wash. The enzymes of the invention together with xanthan lyases may be added to a detergent composition comprising of one or more detergent components.

The invention also relates to methods for degrading xanthan gum on the surface of a textile or hard surface, such as dish wash, comprising applying a composition comprising one or more enzymes of the invention to xanthan gum. The invention further relates to methods for degrading xanthan gum on the surface of a textile or hard surface, such as dish wash, comprising applying a composition comprising one or more xanthan lyase to xanthan gum. An embodiment is a method for degrading xanthan gum on the surface of a textile or hard surface, such as dish wash, comprising applying a composition comprising one or more enzymes of the invention together with one or more xanthan lyase to xanthan gum. An embodiment is the composition comprising one or more detergent components as described above.

Use in the Fracturing of a Subterranean Formation (Oil and/or Gas Drilling)

Hydraulic fracturing is used to create subterranean fractures that extend from the borehole into rock formation in order to increase the rate at which fluids can be produced by the formation. Generally, a high viscosity fracturing fluid is pumped into the well at sufficient pressure to fracture the subterranean formation. In order to maintain the increased exposure to the formation, a solid proppant is added to the fracturing fluid which is carried into the fracture by the high pressure applied to the fluid. Once the high viscosity fracturing fluid has carried the proppant into the formation, breakers are used to reduce the fluid's viscosity which allows the proppant to settle into the fracture and thereby increase the exposure of the formation to the well. Breakers work by reducing the molecular weight of the polymers, thus ‘breaking’ or degrading the polymer. The fracture then becomes a high permeability conduit for fluids and gas to be produced back to the well. Such processes are further disclosed in U.S. Pat. Nos. 7,360,593, 5,806,597, 5,562,160, 5,201,370 and 5,067,566.

Thus the invention relates to the use of an enzyme of the invention as enzyme breakers. An embodiment of the invention is the use of an enzyme of the invention together with a xanthan lyase as enzyme breakers.

Accordingly, the invention provides a method for breaking xanthan gum in a well bore comprising: (i) blending together a gellable fracturing fluid comprising aqueous fluid, one or more hydratable polymers, suitable cross-linking agents for cross-linking the hydratable polymer to form a polymer gel and one or more enzymes of the invention (i.e. the enzyme breaker); (ii) pumping the cross-linked polymer gel into the well bore under sufficient pressure to fracture the surrounding formation; and (iii) allowing the enzyme breaker to degrade the cross-linked polymer to reduce the viscosity of the fluid so that the fluid can be pumped from the formation back to the well surface. As such, the enzymes of the invention can be used to control the viscosity of fracturing fluids. Furthermore, one or more enzymes of the invention together with one or more xanthan lyase can be used to control the viscosity of fracturing fluids.

The enzyme breaker of the present invention may be an ingredient of a fracturing fluid or a breaker-crosslinker-polymer complex which further comprises a hydratable polymer and a crosslinking agent. The fracturing fluid or complex may be a gel or may be gellable. The complex is useful in a method for using the complex in a fracturing fluid to fracture a subterranean formation that surrounds a well bore by pumping the fluid to a desired location within the well bore under sufficient pressure to fracture the surrounding subterranean formation. The complex may be maintained in a substantially non-reactive state by maintaining specific conditions of pH and temperature, until a time at which the fluid is in place in the well bore and the desired fracture is completed. Once the fracture is completed, the specific conditions at which the complex is inactive are no longer maintained. When the conditions change sufficiently, the complex becomes active and the breaker begins to catalyze polymer degradation causing the fracturing fluid to become sufficiently fluid to be pumped from the subterranean formation to the well surface.

Method of Degrading Xanthan Gum Wherein the Xanthan Gum is Used in Fracturing of a Subterranean Formation Perpetrated by a Well Bore

When a well is drilled, reservoir drilling fluid (RDF) is circulated within the drilling equipment to cool down and clean the drill bit, remove the drill cuttings out of the well bore, reduce friction between the drill string and the sides of the borehole, and form a filtercake in order to prevent fluid leak off into the formation. The driving force for the formation of the filtercake is the higher wellbore pressure applied to maintain the borehole stability. This filtercake restricts the inflow of reservoir fluids into the wellbore during the drilling process and placement of the completion. If the filtercake damage that is created during the drilling process is not removed prior to or during completion of the well, a range of issues can arise when the well is put on production, i.e., completion equipment failures and impaired reservoir productivity.

Drilling fluid (mud), also called reservoir drilling fluid (RDF), can be synthetic/oil based or water based. To minimize invasion of the drilling fluid into the formation, both oil based and water based mud filtercakes typically contain a bridging or weighting agent, usually particles of calcium carbonate, barite or a mixture of the two, that bridge at the pore throats of the formation and thereby form a relatively low permeability filtercake. Both oil based and water based mud filtercakes also contain solids called cuttings that have been picked up during drilling, as opposed to the bridging/weighting agents that are added in the formulation of the drilling fluid. These solids can be quartz (sand), silts and/or shales, depending on the reservoir formation as well as the formations traversed by the drilling path to the reservoir. In addition, oil based drilling muds contain water droplets that become trapped in the pore space of the filtercake, while water based mud filtercakes contain polymers, such as starch and xanthan gum, and other inorganic salts.

The formation of a mud filtercake is often necessary for drilling, particularly in unconsolidated formations with wellbore stability problems and typically high permeabilities. The filtercake is then treated with various chemicals, such as chelates or acids to dissolve the calcite component; and/or enzymes or oxidizers to degrade the polymer component to recover permeability.

In one aspect, the invention provides a method for degrading xanthan gum wherein xanthan gum is used in fracturing of a subterranean formation perpetrated by a well bore by applying a composition comprising one of more enzymes of the invention. The method can include the steps of: (i) pumping a treatment fluid comprising one or more enzymes of the invention into the borehole in contact with the filtercake to be removed to establish a differential pressure between the treatment fluid and the formation adjacent the filtercake and (ii) evenly propagating treatment of the filtercake during the differential pressure period to delay breakthrough by the treatment fluid.

In one embodiment, the method can include establishing permeability through the treated filtercake between the formation and the borehole. In another embodiment, the filtercake can include drilling solids and clays, and may be formed from an aqueous drilling fluid. If desired, the treatment fluid for treating the aqueous drilling fluid filtercake can also include an oxidizer and/or a chelate, or it can be substantially free of chelate and oxidizer additives. In another example, the filtercake can be formed from an oil or invert emulsion drilling fluid. If desired, the treatment fluid for treating the oil or invert emulsion drilling fluid filtercake can also include a mutual solvent, a water-wetting agent or a combination thereof to disperse hydrophobic components in the filtercake.

In one embodiment, the treatment fluid comprises one or more GH5 polypeptides of the invention. In another embodiment, the treatment fluid comprises one or more xanthan lyase. In a preferred embodiment, the treatment fluid comprises one or more GH5 polypeptides and one or more xanthan lyase.

Method of Degrading Xanthan Gum Wherein the Xanthan Gum is a Component in a Borehole Filtercake

In one aspect, the invention provides a method for cleaning borehole filtercake, comprising polymers, such as xanthan gum and drilling fluid solids once the filtercake has been pumped to the surface. Drilling mud is pumped from mud pits to the drill bit and then back out to the surface, carrying out amongst other things crushed or cut rock (cuttings) in the process. The cuttings are filtered out and the mud is returned to the mud pits where fines can settle and/or chemicals or enzymes (breakers) can be added.

The method for degrading xanthan gum wherein the xanthan gum is a component in borehole filtercake can include the steps of (i) treating the borehole filtercake with a treatment fluid comprising one or more enzymes of the invention and (ii) separating the solids from the fluids. In a preferred embodiment, the treatment fluid comprises one or more enzymes of the invention and one or more xanthan lyase.

The borehole filtercake may be treated in mud pits with one or more enzymes of the invention and the drilling fluid can be re-circulated. Alternatively, once the filtercake has been treated with one or more enzymes of the invention, the solids and fluid are separated using solid-liquid separation processes, such as centrifugation.

The invention is further defined in the following paragraphs:

1. A polypeptide of glycosyl hydrolase family 5 having xanthan degrading activity.
2. A polypeptide of paragraph 1, selected from the group consisting of:

(a) a polypeptide having at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of any of SEQ ID NO: 2;

(b) a polypeptide encoded by a polynucleotide that hybridizes under medium stringency conditions with (i) the mature polypeptide coding sequence of any of SEQ ID NO: 1, (ii), or the full-length complement of (i);

(c) a polypeptide encoded by a polynucleotide having at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide coding sequence of any of SEQ ID NO: 1;

(d) a variant of the mature polypeptide of any of SEQ ID NO: 2 comprising a substitution, deletion, and/or insertion at one or more positions;

(e) a fragment of the polypeptide of (a), (b), (c), or (d) that has xanthan degrading activity; and

(f) a polypeptide comprising the polypeptide of (a), (b), (c), (d), or (e) and a N-terminal and/or C-terminal His-tag.

3. A polypeptide of paragraph 1, selected from the group consisting of:

(a) a polypeptide having at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of any of SEQ ID NO: 4;

(b) a polypeptide encoded by a polynucleotide that hybridizes under medium stringency conditions with (i) the mature polypeptide coding sequence of any of SEQ ID NO: 3, (ii), or the full-length complement of (i);

(c) a polypeptide encoded by a polynucleotide having at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide coding sequence of any of SEQ ID NO: 3;

(d) a variant of the mature polypeptide of any of SEQ ID NO: 4 comprising a substitution, deletion, and/or insertion at one or more positions;

(e) a fragment of the polypeptide of (a), (b), (c), or (d) that has xanthan degrading activity; and

(f) a polypeptide comprising the polypeptide of (a), (b), (c), (d), or (e) and a N-terminal and/or C-terminal His-tag.

4. A polypeptide of paragraph 1, selected from the group consisting of:

(a) a polypeptide having at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of any of SEQ ID NO: 6;

(b) a polypeptide encoded by a polynucleotide that hybridizes under medium stringency conditions with (i) the mature polypeptide coding sequence of any of SEQ ID NO: 5, (ii), or the full-length complement of (i);

(c) a polypeptide encoded by a polynucleotide having at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide coding sequence of any of SEQ ID NO: 5;

(d) a variant of the mature polypeptide of any of SEQ ID NO: 6 comprising a substitution, deletion, and/or insertion at one or more positions;

(e) a fragment of the polypeptide of (a), (b), (c), or (d) that has xanthan degrading activity; and

(f) a polypeptide comprising the polypeptide of (a), (b), (c), (d), or (e) and a N-terminal and/or C-terminal His-tag.

5. A polypeptide of paragraph 1, selected from the group consisting of:

(a) a polypeptide having at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of any of SEQ ID NO: 8;

(b) a polypeptide encoded by a polynucleotide that hybridizes under medium stringency conditions with (i) the mature polypeptide coding sequence of any of SEQ ID NO: 7, (ii), or the full-length complement of (i);

(c) a polypeptide encoded by a polynucleotide having at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide coding sequence of any of SEQ ID NO: 7;

(d) a variant of the mature polypeptide of any of SEQ ID NO: 8 comprising a substitution, deletion, and/or insertion at one or more positions;

(e) a fragment of the polypeptide of (a), (b), (c), or (d) that has xanthan degrading activity; and

(f) a polypeptide comprising the polypeptide of (a), (b), (c), (d), or (e) and a N-terminal and/or C-terminal His-tag.

6. The polypeptide of any of paragraphs 1 to 5, having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of any of SEQ ID NO: 2, 4, 6, or 8.
7. The polypeptide of any of paragraphs 1 to 6, which is encoded by a polynucleotide that hybridizes under medium-high stringency conditions with (i) the mature polypeptide coding sequence of any of SEQ ID NO: 1, 3, 5, or 7, or (ii) the full-length complement of (i).
8. The polypeptide of any of paragraphs 1 to 7, which is encoded by a polynucleotide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide coding sequence of any of SEQ ID NO: 1, 3, 5, or 7.
9. The polypeptide of any of paragraphs 1 to 8, consisting of any of SEQ ID NO: 2, 4, 6, or 8. or the mature polypeptide of any of SEQ ID NO: 2, 4, 6, or 8.
10. The polypeptide of any of paragraphs 1 to 9, comprising any of SEQ ID NO: 2, 4, 6, or 8 or the mature polypeptide of any of SEQ ID NO: 2, 4, 6, or 8.
11. The polypeptide of any of paragraphs 1 to 10, which is a variant of the mature polypeptide of any of SEQ ID NO: 2, 4, 6, or 8 comprising a substitution, deletion, and/or insertion at one or more positions, such as up to 10, e.g., 1, 2, 3, 4, 5, 6, 7, 8, 9, or 10 positions.
12. The polypeptide of paragraphs 1 to 11, which is a fragment of any of SEQ ID NO: 2, 4, 6, or 8, wherein the fragment has xanthan degrading activity.
13. A polynucleotide encoding the polypeptide of any of paragraphs 1-12.
14. A nucleic acid construct or expression vector comprising the polynucleotide of paragraph 13 operably linked to one or more control sequences that direct the production of the polypeptide in an expression host.
15. A recombinant host cell comprising the polynucleotide of paragraph 13 operably linked to one or more control sequences that direct the production of the polypeptide.
16. A method of producing the polypeptide of any of paragraphs 1-12, comprising cultivating a cell, which in its wild-type form produces the polypeptide, under conditions conducive for production of the polypeptide.
17. The method of paragraph 16, further comprising recovering the polypeptide.
18. A method of producing a polypeptide having activity on xanthan gum, comprising cultivating the host cell of paragraph 15 under conditions conducive for production of the polypeptide.
19. A transgenic plant, plant part or plant cell transformed with a polynucleotide encoding the polypeptide of any of paragraphs 1-12
20. A method of producing a polypeptide having activity on xanthan gum, comprising cultivating the transgenic plant or plant cell of paragraph 19 under conditions conducive for production of the polypeptide.
21. The method of paragraph 20, further comprising recovering the polypeptide.
22. A whole broth formulation or cell culture composition comprising a polypeptide of any of paragraphs 1-12.
23. A composition comprising the polypeptide of any of paragraphs 1-12.
24. The composition of paragraph 23 further comprising a polypeptide having xanthan lyase activity.
25. The composition of paragraph 24 wherein the polypeptide having xanthan lyase activity is a polypeptide having the amino acid sequence of any one of SEQ ID NO: 21, 22, 23 or 24.
26. Use of a composition according to any of paragraphs 23 to 25 for degrading xanthan gum.
27. The use of paragraph 30 for controlling the viscosity of a drilling fluid.
28. A method for degrading xanthan gum comprising applying a composition according to any of paragraphs 23 to 25 to xanthan gum.
29. The method of paragraph 28, wherein the xanthan gum is on the surface of a textile or of a hard surface, such as in dish wash.
30. The method of paragraph 28, wherein the xanthan gum is used in fracturing of a subterranean formation penetrated by a well bore.
31. The method of paragraph 28, wherein the xanthan gum is a component in a borehole filtercake.
32. A method for degrading or converting a cellulosic material, comprising: treating the cellulosic material with the enzyme composition according to any of paragraphs 23 to 25 or in the presence of the polypeptide of any of paragraphs 1 to 12.
33. The method of paragraph 32, wherein the cellulosic material is pretreated.
34. The method of paragraph 32 or 33, wherein the enzyme composition comprises one or more enzymes selected from the group consisting of a cellulase, a polypeptide having cellulolytic enhancing activity, a hemicellulase, an esterase, a protease, a laccase, or a peroxidase.
35. The method of paragraph 34, wherein the cellulase is one or more enzymes selected from the group consisting of an endoglucanase, a cellobiohydrolase, and a beta-glucosidase.
36. The method of paragraph 35, wherein the hemicellulase is one or more enzymes selected from the group consisting of a xylanase, an acetyxylan esterase, a feruloyl esterase, an arabinofuranosidase, a xylosidase, and a glucuronidase.
37. The method of any of paragraphs 32 to 36, further comprising recovering the degraded cellulosic material.
38. The method of paragraph 37, wherein the degraded cellulosic material is a sugar, preferably selected from the group consisting of glucose, xylose, mannose, galactose, and arabinose.
39. A method for producing a fermentation product, comprising:

(a) saccharifying a cellulosic material in the presence of the polypeptide of any of paragraph 1-13 or the enzyme composition according to any of paragraphs 23 to 25; (b) fermenting the saccharified cellulosic material with one or more fermenting microorganisms to produce the fermentation product; and

(c) recovering the fermentation product from the fermentation.

The present invention is further described by the following examples that should not be construed as limiting the scope of the invention.

### EXAMPLES

### Activity Assays

Xanthan Lyase Activity Assay

0.8 mL 100 mM HEPES buffer, pH 6.0 was mixed with 0.2 mL Xanthan gum (5 mg/mL) dissolved in water in a 1 mL 1 cm cuvette. The cuvette was inserted into a spectrophotometer (Agilent G1103A 8453A, CA, USA) with temperature control set at 40° C. The solution was pre-incubated for 10 min and 0.1 mL sample was added and the solution was mixed by aspiring and dispensing the solution for at least 5 times using a pipette. Total reaction volume was 1.1 mL.

Absorbance at 235 nm was collected for 10 min using a 30 sec measuring interval. Initial activity was calculated by using the software (UV-Visible Chemstation Rev A.10.01 [81], Agilent).

### Example 1: Strain and DNA

The DNA in SEQ ID NO: 1 encoding the GH5 polypeptide EXa of SEQ ID NO: 2 was obtained from an Opitutaceae species isolated from an environmental soil sample collected in Denmark.

The DNA SEQ ID NO: 3 encoding the GH5 polypeptide EXb of SEQ ID NO: 4 was isolated from an environmental sample collected in Denmark.

The DNA SEQ ID NO: 5 encoding the GH5 polypeptide EXc of SEQ ID NO: 5 was isolated from an environmental sample collected in Denmark.

The DNA SEQ ID NO: 7 encoding the GH5 polypeptide EXd of SEQ ID NO: 8 was obtained from the public database (UNIPROT M2V1S3) but originates from a strain of Pseudomonas stutzeri collected from a Galapagos Rift hydrothermal vent, Ecuador.

Codon optimized synthetic DNA encoding the mature peptide sequences of the four polypeptides were prepared (SEQ ID NO: 9; SEQ ID NO: 10, SEQ ID NO: 11; SEQ ID NO: 12).

### Example 2: Cloning and Expression of GH5 Polypeptides

The GH5 encoding genes were either cloned by conventional techniques from the strains indicated above or from the synthetic DNA and inserted into a suitable plasmid as described below.

### Example 2a: Cloning and Expression of GH5 Polypeptides in E. coli

The mature peptide encoding part of the GH5 endo-glucanase genes, SEQ ID NO: 1, 3, 5 and 7 was inserted with an N-terminal poly histidine tag with an extra proline and arginine (HHHHHHPR) (SEQ ID NO: 19) after the methionine in the E. coli pET-32a(+) vector from Novagen with standard recombinant techniques. The expression plasmid containing the insert was purified from an E. coli transformant harboring the plasmid and transformed into E. coli Xjb (DE3) host cells (from Zymo Research). A fresh clone of E. coli Xjb (DE3) containing the pET32-GH5 vector, was grown overnight in Terrific Broth containing 100 ug/ml ampicillin. Next day, a fresh 500 ml culture was inoculated with 1 ml overnight culture and cells were cultured (37° C., 250 rpm) to an optical density (OD600) between 6-8. Protein expression was induced by 1 mM isopropylthio-D-galactosidase (IPTG) and 6 mM arabinose for 4.5 hours at 20° C. After continued culture, cells were harvested by centrifugation and lysed by Bugbuster® (Novagen).

The soluble fraction was used for polyhistidine tag purification of the GH5 polypeptides SEQ ID NO: 13, 14 and 15 as described in example 4.

### Example 2b: Cloning and Expression of GH5 Polypeptides in Bacillus subtilis

The synthetic codon optimized genes SEQ ID NO: 10, 11 and 12 were cloned into the Bacillus expression vector described in WO 2012/025577. The genes were expressed by replacing the native secretion signal sequence with the Bacillus clausii secretion signal MKKPLGKIVASTALLISVAFSSSIASA (SEQ ID NO: 20) with an extra affinity tag sequence (HHHHHHPR) (SEQ ID NO: 19) at the C-terminal of the signal peptide, to facilitate the purification process. This resulted in a recombinant mature polypeptide with a His tag at the front of the N-terminal of the mature wild type sequence (SEQ ID NO: 16, 17 and 18).

One clone with the correct recombinant gene sequence was selected and the corresponding plasmid was integrated by homologous recombination into the Bacillus subtilis host cell genome (pectate lyase locus) and the gene construct was expressed under the control of a triple promoter system as described in WO99/43835. The gene coding for chloramphenicol acetyltransferase was used as a marker (as described in Diderichsen et al., 1993, Plasmid 30:312-315).

Chloramphenicol resistant transformants were analyzed by PCR to verify the correct size of the amplified fragment. A recombinant B. subtilis clone containing the integrated expression construct was selected and cultivated on a rotary shaking table in 500 mL baffled Erlenmeyer flasks each containing 100 ml yeast extract-based media. The clone was cultivated for 5 days at 30° C. The enzyme containing supernatants were harvested and the enzyme purified as described in Example 5.

### Example 3: Purification of Wild Type GH5 Polypeptide from the Natural Opitutaceae Strain

The Opitutaceae strain was cultivated on a rotary shaking table in 500 mL baffled Erlenmeyer flasks each containing 100 ml mineral solution with 0.5% xanthan gum. The strain was cultivated for 20 days at 30° C. A total of 2.0 L supernatant was harvested by centrifugation and was filtered using a 0.2 μm bottle top filter (Nalgene Nunc). The broth was concentrated to 300 mL using ultra-filtration (Sartorius) with 30 kDa cut-off. Equal volume of 3.2 M ammonium sulphate in 40 mM Tris-HCl, pH 7.9 was slowly added with continuous stirring. The sample was filtered using Whatman glass filters (1.7 μm-0.7 μm) to remove larger particles. The sample was applied on a 20 mL Phenyl-sepharose high performance column (GE Healthcare) pre-equilibrated with 1.6 M ammonium sulphate in 20 mM Tris-HCl, pH 7.9 (equilibration buffer). Unbound protein was eluted by two column volumes of equilibration buffer. Elution was done by a 12 column volume linear gradient from 1.6 M to 0.0 M ammonium sulphate in 20 mM Tris-HCl, pH 7.9. A last elution step of 4 column volume with equilibration buffer was used to elute tightly bound protein. The absorbance at 280 nm was recorded during the entire purification. Protein containing fractions identified by the absorbance at 280 nm in the chromatogram were analyzed by SDS-PAGE (NuPAGE, Invitrogen). Fractions judged as pure were pooled. The sample was concentration from 30 to 4 mL using Macrosep ultra filtration device with 3 kDa cut-off (Pall). The protein concentration was determined by measuring the absorbance at 280 nm using the calculated extinction coefficient where 1 mg/mL equaled 1.89 absorbance units.

### Example 4: Purification of Recombinant GH5 Polypeptide Produced in E. coli

200 mL lysed cells (grown as example 2a) were filtered through Fast PES 0.2 μm bottle-top filters to remove debris and unbroken cells. 200 mL of equilibration buffer (20 mM Tris-HCl, pH 7.5+500 mM NaCl) was added to the crude protein solution. A 20 mL HisPrep column loaded with Ni²⁺ was equilibrated with equilibration buffer until a stable UV baseline was obtained. The absorbance at 280 nm was continuously monitored throughout the purification. Crude protein was loaded on the column using a flow rate of 4 mL/min. Unbound protein was removed by washing the column with equilibration buffer until a stable UV baseline was obtained. Elution was carried out by a two-step linear gradient using 20 mM Tris-HCl, pH 7.5+500 mM NaCl+500 mM Imidazole (elution buffer). First elution gradient was 10 column volumes 0 to 40% elution buffer followed by 4 column volumes from 40% to 100%. Peaks absorbing at 280 nm were analyzed by SDS-PAGE (NuPAGE, Invitrogen). Fractions containing protein with the correct apparent molecular weight were pooled. The pool was desalted and buffer exchanged using a Sephadex G-25 super fine desalting column equilibrated with 20 mM Tris-HCl, pH 8.0. The pool was applied on a 20 mL Sourcel5Q column pre-equilibrated with 20 mM Tris-HCl, pH 8.0. Unbound protein was washed out using 20 mM Tris-HCl, pH 8.0 until a stable UV baseline was obtained. Elution was done by a 10 column volume linear NaCl gradient from 0 to 500 mM NaCl in 20 mM Tris-HCl, pH 8.0. Protein containing fractions were analyzed by SDS-PAGE and fractions judged as pure were pooled. Protein concentration was measured using absorbance at 280 nm using a calculated extinction coefficient where 1 mg/mL corresponded to 1.86 absorbance units.

### Example 5: Purification of Recombinant GH5 Polypeptide Produced in B. subtilis

All His-tagged enzymes were purified by immobilized metal chromatography (IMAC) using Ni²⁺ as the metal ion on 5 mL HisTrap Excel columns (GE Healthcare Life Sciences). The purification was done at pH 8 and the bound proteins were eluted with imidazole. The purity of the purified enzymes was checked by SDS-PAGE and the concentration of each enzyme determined by Abs 280 nm after a buffer exchange.

### Example 6: Xanthan Degrading Activity of GH5 Polypeptide and Xanthan Lyase on Xanthan Gum by Measurement of Viscosity Reduction

The viscosity reduction measurements were performed using the viscosity pressure assay described in WO2011/107472 and following the method described in WO2013167581. Results presented are the average of three measurements and are shown in table 1 and 2 below.

A sample size of was 400 μL was used. The hydrolysis conditions were as follows: 30° C., either 0.25% or 0.5% xanthan gum (XG) in 50 mM MES buffer+0.01% triton x-100 pH 7.0 or 100 mM CHES buffer+0.01% triton x-100 pH10. Enzyme was added upon thermal equilibration. Prior to use all enzymes were buffer changed to the MES buffer using NAP 5 columns (GE Healthcare).

The purified enzyme preparations of Example 5 were used for the analysis at a concentration of 31.25 mg/L.

TABLE 1
Viscosity measurements (Pa) of EXa (SEQ ID NO: 13) and/or
Xanthan Lyase (SEQ ID NO: 21) on 0.5% xanthan gum at pH 7.

| | T = 0 | T = 30 | T = 1 | T = 2 | T = 3 | T = 4 |
|---|---|---|---|---|---|---|
| | minutes | minutes | hour | hours | hours | hours |
| Water (control) | 430 ± 44 | 504 ± 50 | 470 ± 75 | 483 ± 86 | 466 ± 60 | 504 ± 82 |
| Xanthan gum (control) | 1703 ± 132 | 1738 ± 26 | 1837 ± 122 | 1803 ± 64 | 1739 ± 84 | 1757 ± 21 |
| Xanthan gum + EXa | 1586 ± 101 | 1154 ± 38 | 1270 ± 67 | 1230 ± 36 | 1156 ± 49 | 1184 ± 44 |
| SEQ ID NO: 13 | | | | | | |
| Xanthan gum + XLa | 1963 ± 93 | 1884 ± 67 | 1890 ± 84 | 1840 ± 131 | 1886 ± 50 | 1950 ± 25 |
| SEQ ID NO: 21 | | | | | | |
| Xanthan gum + EXa | 1370 ± 197 | 861 ± 23 | 973 ± 59 | 840 ± 62 | 916 ± 47 | 904 ± 79 |
| SEQ ID NO: 13 + XLa | | | | | | |
| SEQ ID NO: 21 | | | | | | |

The results presented above show that the GH5 polypeptide alone and in combination with xanthan lyase can degrade the xanthan gum present in the media at pH 7, thus leading to viscosity reduction. A synergistic effect is obtained with combination of GH5 and xanthan lyase.

TABLE 2
Viscosity measurements (Pa) of EXa (SEQ ID NO: 13) and/or
Xanthan Lyase (SEQ ID NO: 23) on 0.5% xanthan gum at pH 10

| | | T = 0.5 | T = 1 | T = 2 | T = 3.5 |
|---|---|---|---|---|---|
| | T = 0 | hours | hours | hours | hours |
| Water | 370 ± 10 | 454 ± 15 | 519 ± 60 | 411 ± 29 | 554 ± 180 |
| Xanthan gum | 1740 ± 151 | 1734 ± 21 | 1819 ± 67 | 1795 ± 29 | 1898 ± 75 |
| (XG) control | | | | | |
| XG + EXa | 1676 ± 50 | 1324 ± 58 | 1223 ± 12 | 1251 ± 31 | 1318 ± 62 |
| SEQ ID NO: 13 | | | | | |
| XG + XLc | 2046 ± 112 | 1811 ± 82 | 1773 ± 64 | 1781 ± 92 | 1704 ± 67 |
| SEQ ID NO: 23 | | | | | |
| XG + EXa SEQ | 1573 ± 227 | 1057 ± 21 | 1153 ± 12 | 1161 ± 40 | 1188 ± 89 |
| ID NO: 13 + XLc | | | | | |
| SEQ ID NO: 23 | | | | | |

The results presented above show that the GH5 polypeptide in alone or combination with xanthan lyase can degrade the xanthan gum present in the media at pH 10, thus leading to viscosity reduction.

TABLE 3
Viscosity measurements (Pa) of EXa (SEQ ID NO:
13), EXd (SEQ ID NO: 18) and/or Xanthan Lyase (XLa,
SEQ ID NO: 21) on 0.5% xanthan gum at pH 7.

| | | T = 0.5 | T = 1 | T = 2 | T = 3 |
|---|---|---|---|---|---|
| | T = 0 | hours | hours | hours | hours |
| Water control | 440 | 410 | 333 | 413 | 469 |
| Xanthan gum | 1626 | 1590 | 1546 | 1566 | 1659 |
| (XG) control | | | | | |
| XG + EXa | 1220 | 1080 | 1046 | 1040 | 1079 |
| SEQ ID NO: 13 | | | | | |
| XG + EXa SEQ | 1263 | 850 | 786 | 793 | 815 |
| ID NO: 13 + XLa | | | | | |
| SEQ ID NO: 21 | | | | | |
| XG + EXd | 1476 | 1406 | 1313 | 1283 | 1245 |
| SEQ ID NO: 18 | | | | | |
| XG + EXd SEQ | 1490 | 1056 | 1023 | 933 | 912 |
| ID NO: 18 + XLa | | | | | |
| SEQ ID NO: 21 | | | | | |

The results presented above show that the GH5 polypeptide alone and in combination with xanthan lyase can degrade the xanthan gum present in the media at pH 7, thus leading to viscosity reduction.

TABLE 4
Viscosity measurements (Pa) of EXa, EXb, EXc recombinantly expressed in E.coli (SEQ ID NO: 13; SEQ
ID NO: 14, SEQ ID NO: 15) and/or Xanthan Lyase (XLb, SEQ ID NO: 22) on 0.5% xanthan gum at pH 7.

| | | | T = 30 | T = 1 | T = 2 | T = 3 | T = 4 |
|---|---|---|---|---|---|---|---|
| | T = 00 | T = 0 | min | hr | hrs | hrs | hrs |
| Water | 541 ± 21 | 544 ± 119 | 519 ± 142 | 545 ± 70 | 399 ± 80 | 422 ± 114 | 326 ± 25 |
| Xanthan gum | 1878 ± 20 | 1444 ± 15 | 1599 ± 91 | 1571 ± 64 | 1605 ± 38 | 1586 ± 40 | 1566 ± 32 |
| control | | | | | | | |
| XG + XLb | 1898 ± 26 | 1511 ± 12 | 1522 ± 56 | 1505 ± 20 | 1579 ± 80 | 1516 ± 21 | 1559 ± 38 |
| SEQ ID NO: 22 | | | | | | | |
| XG + EXb | 1884 ± 31 | 1281 ± 55 | 1202 ± 120 | 1145 ± 52 | 1132 ± 70 | 1096 ± 60 | 1116 ± 114 |
| SEQ ID NO: 14 | | | | | | | |
| XG + EXc | 1931 ± 45 | 1444 ± 80 | 1122 ± 36 | 1108 ± 42 | 1105 ± 45 | 1019 ± 10 | 1059 ± 15 |
| SEQ ID NO: 15 | | | | | | | |
| XG + EXa | 1891 ± 12 | 1441 ± 38 | 1102 ± 17 | 1051 ± 25 | 1005 ± 6 | 969 ± 26 | 1036 ± 25 |
| SEQ ID NO: 13 | | | | | | | |
| XG + EXb SEQ | 1918 ± 61 | 1121 ± 6 | 862 ± 17 | 731 ± 31 | 689 ± 25 | 652 ± 40 | 576 ± 40 |
| ID NO: 14 + XLb | | | | | | | |
| SEQ ID NO: 22 | | | | | | | |
| XG + EXc SEQ | 1911± | 1111± | 935± | 848± | 832± | 822± | 789± |
| ID NO: 15 + XLb | | | | | | | |
| SEQ ID NO: 22 | | | | | | | |
| XG + EXa SEQ | 1934 ± 31 | 1198 ± 36 | 855 ± 40 | 831 ± 40 | 785 ± 23 | 909 ± 26 | 819 ± 64 |
| ID NO: 13 + XLb | | | | | | | |
| SEQ ID NO: 22 | | | | | | | |

T = 00 is before addition of enzyme and T = 0 is right after.

The results presented above show that the GH5 polypeptides EXa, EXb and EXc alone and in combination with xanthan lyase can degrade the xanthan gum present in the media at pH 7, thus leading to viscosity reduction. A synergistic effect is obtained with combination of GH5 polypeptide and xanthan lyase

TABLE 5
Viscosity measurements (Pa) of EXa, recombinantly expressed in E. coli (SEQ ID NO:
13) and EXb and EXc recombinantly expressed in B. subtilis (SEQ ID NO: 16 and SEQ
ID NO: 17) and/or Xanthan Lyase (XLb, SEQ ID NO: 22) on 0.5% xanthan gum at pH 7.

| | | | T = 30 | T = 1 | T = 2 | T = 3 | T = 4 |
|---|---|---|---|---|---|---|---|
| | T = 00 | T = 0 | min | hour | hours | hours | hours |
| Water | 441 ± 25 | 421 ± 40 | 646 ± 44 | 535 ± 59 | 599 ± 74 | 492 ± 15 | 494 ± 32 |
| Xanthan | 2027 ± 23 | 1707 ± 35 | 1949 ± 59 | 1785 ± 116 | 1746 ± 75 | 1726 ± 10 | 1867 ± 6 |
| gum(XG) | | | | | | | |
| XG + EXa | 2054 ± 44 | 1514 ± 17 | 1299 ± 21 | 1112 ± 57 | 1089 ± 45 | 1046 ± 0 | 1027 ± 6 |
| SEQ ID NO: 13 | | | | | | | |
| XG + EXb | 2067 ± 15 | 1527 ± 81 | 1393 ± 12 | 1229 ± 53 | 1159 ± 12 | 1136 ± 0 | 1134 ± 6 |
| SEQ ID NO: 16 | | | | | | | |
| XG + EXc | 2061 ± 31 | 1501 ± 55 | 1416 ± 44 | 1175 ± 6 | 1183 ± 78 | 1169 ± 40 | 1147 ± 15 |
| SEQ ID NO: 17 | | | | | | | |
| XG + EXa SEQ | 2061 ± 6 | 1274 ± 17 | 1063 ± 47 | 812 ± 59 | 769 ± 46 | 729 ± 15 | 671 ± 26 |
| ID NO: 13 + XLb | | | | | | | |
| SEQ ID NO: 22 | | | | | | | |
| XG + EXb SEQ | 2074 ± 26 | 1411 ± 65 | 1079 ± 15 | 945 ± 92 | 809 ± 12 | 796 ± 10 | 781 ± 10 |
| ID NO: 20 + XLb | | | | | | | |
| SEQ ID NO: 22 | | | | | | | |
| XG + EXc SEQ | 2094 ± 30 | 1491 ± 25 | 1166 ± 0 | 959 ± 46 | 889 ± 40 | 846 ± 0 | 847 ± 57 |
| ID NO: 17 + XLb | | | | | | | |
| SEQ ID NO: 22 | | | | | | | |
| XG + XLb | 2097 ± 49 | 1794 ± 62 | 1863 ± 23 | 1685 ± 15 | 1653 ± 10 | 1679 ± 6 | 1667 ± 29 |
| SEQ ID NO: 22 | | | | | | | |
| XG + EXa SEQ | 2131 ± 15 | 1227 ± 81 | 1143 ± 81 | 789 ± 62 | 739 ± 25 | 716 ± 44 | 677 ± 55 |
| ID NO: 13 + XLa | | | | | | | |
| SEQ ID NO: 21 | | | | | | | |
| XG + EXb SEQ | 2104 ± 79 | 1324 ± 17 | 1096 ± 44 | 795 ± 31 | 803 ± 26 | 792 ± 21 | 767 ± 12 |
| ID NO: 16 + XLa | | | | | | | |
| SEQ ID NO: 21 | | | | | | | |
| XG + EXc SEQ | 2107 ± 12 | 1241 ± 50 | 1163 ± 32 | 802 ± 15 | 826 ± 15 | 846 ± 0 | 894 ± 15 |
| ID NO: 17 + XLa | | | | | | | |
| SEQ ID NO: 21 | | | | | | | |
| XG + XLa | 2134 ± 20 | 1741 ± 57 | 1933 ± 29 | 1639 ± 30 | 1659 ± 23 | 1666 ± 17 | 1637 ± 12 |
| SEQ ID NO: 21 | | | | | | | |

T = 00 is before addition of enzyme and T = 0 is right after.

The results presented above show that the GH5 polypeptides EXa, EXb and EXc alone and in combination with xanthan lyase can degrade the xanthan gum present in the media at pH 7, thus leading to viscosity reduction. A synergistic effect is obtained with combination of GH5 polypeptide and xanthan lyase.

TABLE 6
Viscosity measurements (Pa) of EXa, EXb, EXc recombinantly expressed in
E. coli (SEQ ID NO: 13; SEQ ID NO: 14 or SEQ ID NO: 15) and/or Xanthan
Lyase (XLc, SEQ ID NO: 23 or SEQ ID NO: 24) on 0.5% xanthan gum at pH 10.

| | | | | T = 1 | T = 2 | T = 3 |
|---|---|---|---|---|---|---|
| | T = 00 | T = 0 | T = 30′ | hr | hrs | hrs |
| Water | 429 ± 66 | 502 ± 110 | 504 ± 50 | 434 ± 29 | 478 ± 42 | 479 ± 26 |
| Xanthan | 1932 ± 31 | 1485 ± 81 | 1678 ± 12 | 1641 ± 70 | 1642 ± 38 | 1592 ± 92 |
| gum (XG) | | | | | | |
| XG + EXa | 1992 ± 138 | 1332 ± 6 | 1254 ± 21 | 1147 ± 51 | 1192 ± 35 | 1215 ± 31 |
| SEQ ID NO: 13 | | | | | | |
| XG + EXb | 1989 ± 85 | 1415 ± 50 | 1351 ± 66 | 1321 ± 17 | 1358 ± 51 | 1252 ± 21 |
| SEQ ID NO: 14 | | | | | | |
| XG + EXc | 1892 ± 45 | 1442 ± 100 | 1408 ± 21 | 1341 ± 50 | 1332 ± 31 | 1262 ± 51 |
| SEQ ID NO: 17 | | | | | | |
| XG + EXa SEQ | 1899 ± 69 | 1429 ± 62 | 1084 ± 76 | 1131 ± 17 | 1092 ± 25 | 1112 ± 40 |
| ID NO: 13 + XLc | | | | | | |
| SEQ ID NO: 23 | | | | | | |
| XG + EXb SEQ | 2019 ± 62 | 1465 ± 132 | 1144 ± 23 | 1121 ± 53 | 1108 ± 81 | 1012 ± 59 |
| ID NO: 14 + XLc | | | | | | |
| SEQ ID NO: 23 | | | | | | |
| XG + EXc SEQ | 2085 ± 80 | 1602 ± 38 | 1344 ± 15 | 1321 ± 10 | 1262 ± 55 | 1319 ± 10 |
| ID NO: 15 + XLc | | | | | | |
| SEQ ID NO: 23 | | | | | | |
| XG + XLc | 2005 ± 47 | 1702 ± 75 | 1588 ± 6 | 1524 ± 67 | 1588 ± 60 | 1569 ± 36 |
| SEQ ID NO: 23 | | | | | | |
| XG + EXa SEQ | 1959 ± 72 | 1462 ± 110 | 1158 ± 38 | 1144 ± 40 | 1148 ± 72 | 1005 ± 45 |
| ID NO: 13 + XLd | | | | | | |
| SEQ ID NO: 24 | | | | | | |
| XG + EXb SEQ | 1975 ± 25 | 1442 ± 35 | 1211 ± 26 | 1177 ± 15 | 1192 ± 72 | 1182 ± 67 |
| ID NO: 14 + XLd | | | | | | |
| SEQ ID NO: 24 | | | | | | |
| XG + EXc SEQ | 1925 ± 133 | 1422 ± 95 | 1238 ± 12 | 1274 ± 58 | 1208 ± 81 | 1215 ± 67 |
| ID NO: 15 + XLd | | | | | | |
| SEQ ID NO: 24 | | | | | | |
| XG + XLd | 1839 ± 40 | 1525 ± 61 | 1488 ± 21 | 1447 ± 42 | 1432 ± 15 | 1425 ± 76 |
| SEQ ID NO: 24 | | | | | | |

T = 00 is before addition of enzyme and T = 0 is right after.

The results presented above show that the GH5 polypeptides GH5, EXb and EXc in combination with xanthan lyase can degrade the xanthan gum present in the media at pH 10, thus leading to viscosity reduction.

TABLE 7
Viscosity measurements (Pa) of GH5 polypeptide purified from
supernatant of the Opitutaceae sp strain and/or Xanthan Lyase
(XLa, SEQ ID NO: 21) on 0.25% xanthan gum at pH 7

| | | T = 0.5 | T = 1 | T = 2 | T = 3 |
|---|---|---|---|---|---|
| | T = 0 | hour | hour | hours | hours |
| Water | 471 ± 99 | 390 ± 46 | 423 ± 61 | 433 ± 64 | 438 ± 36 |
| Xanthan | 898 ± 12 | 880 ± 40 | 900 ± 17 | 820 ± 40 | 908 ± 50 |
| gum (XG) | | | | | |
| XG + | 856 ± 34 | 743 ± 46 | 723 ± 34 | 672 ± 38 | 644 ± 55 |
| EXa SEQ | | | | | |
| ID NO: 1 | | | | | |
| XG + | 908 ± 29 | 865 ± 22 | 860 ± 35 | 857 ± 32 | 856 ± 61 |
| XLa SEQ | | | | | |
| ID NO: 21 | | | | | |
| XG + | 800 ± 28 | 597 ± 30 | 612 ± 31 | 577 ± 45 | 648 ± 89 |
| EXa SEQ | | | | | |
| ID NO: 1 + | | | | | |
| XLa SEQ | | | | | |
| ID NO: 21 | | | | | |

### Example 8: Xanthan Degrading Activity of GH5 Polypeptide and Xanthan Lyase on Xanthan Gum by Measurement of Viscosity Reduction

The viscosity measurements were performed using the viscosity pressure assay described in WO2011/107472. 150 μL of each 1 mL hydrolysis or control was the sample size. Results presented are the average of four measurements and are shown in table 8 and 9 below.

Modified xanthan gum was prepared by an adaption of Nankai et al. 1999. “Microbial system for polysaccharide depolymerization: enzymatic route for xanthan depolymerization by Bacillus sp strain GL1.” Applied and Environmental Microbiology 65(6): 2520-2526.

2.5 g of xanthan gum (CP Kelco) was wetted with 5 mL of 96% ethanol in a 2 L beaker. 500 mL of 100 mM ACES buffer pH 7.00 was added and the solution stirred at ambient temperature for 2 h. 250 μL of xanthan lyase (Bacillus sp., Megazyme) was added and the solution incubated for 20 h at 50° C. The sample was then cooled by placing the beaker on ice.

After hydrolysis was 1400 mL of ice cold 96% ethanol was added to the 500 mL sample, under stirring. Precipitation occurs, and after approximately 5 min the ethanol was decanted removing the pyruvated mannose residues. The sample was vacuum filtered and transferred to a glass plate. The glasses were dried at 50° C. for 20 h. The sample was collected, weighed, and grinded.

The hydrolysis conditions were as follows: 40° C., 0.35% xanthan gum (XG) in 50 mM HEPES buffer+0.01% triton X-100 pH 7.0. The modified xanthan gum powder (mXG) was prepared as described above and a 0.7% solution was prepared using the same procedure as outlined for XG. Enzyme was added upon thermal equilibration. The initial viscosity is measured prior to enzyme addition, after thermal equilibration. Controls are the same with buffer added instead of enzyme. Buffer was monitored to determine the ultimate end point of a total hydrolysis.

TABLE 8
Viscosity measurements (Pa). EXc SEQ ID NO: 17 and XLb
(SEQ ID NO: 22). Each enzyme dosed in 1.5 ppm. pH 7.0

| Time (Minutes) | 0 | 15 | 30 | 45 | 60 | 75 | 90 |
|---|---|---|---|---|---|---|---|
| Buffer 50 mM | 645 | 610 | 521 | 502 | 620 | 632 | 600 |
| HEPES Control | | | | | | | |
| Xanthan Gum + | 2140 | 2075 | 1948 | 2092 | 2033 | 2077 | 2005 |
| Buffer Control | | | | | | | |
| Xanthan Gum + | 2120 | 1295 | 991 | 957 | 935 | 1112 | 917 |
| EXc | | | | | | | |
| Xanthan Gum + | 1977 | 808 | 811 | 837 | 773 | 807 | 777 |
| EXc + | | | | | | | |
| Xanthan Lyase | | | | | | | |
| Xanthan Gum + | 1972 | 1853 | 1838 | 1802 | 1750 | 1737 | 1677 |
| Xanthan lyase | | | | | | | |
| Modified | 2262 | 2100 | 2143 | 2134 | 2118 | 2150 | 2097 |
| Xanthan Gum + | | | | | | | |
| Buffer Control | | | | | | | |
| Modified | 2217 | 1225 | 1173 | 1157 | 1130 | 1155 | 1130 |
| Xanthan Gum + | | | | | | | |
| EXc | | | | | | | |

### Example 9: Wash Performance of GH5 Polypeptide and Xanthan Lyase

The wash performance of the GH5 enzyme was assessed in laundry wash experiments using a Mini wash assay, which is a test method where soiled textile is continuously lifted up and down into the test solution and subsequently rinsed. The wash experiment was conducted under the experimental conditions specified in Table 10.

The textiles were subsequently air-dried and the wash performance was measured as the brightness of the color of the textiles. Brightness can be expressed as the Remission (R), which is a measure for the light reflected or emitted from the test material when illuminated with white light. The Remission (R) of the textiles was measured at 460 nm using a Zeiss MCS 521 VIS spectrophotometer. The measurements were done according to the manufacturer's protocol.

The performance of the new enzyme (combination) was compared to the performance of detergent alone (blank). An enzyme (combination) is considered to exhibit improved wash performance, if it performs better than the detergent alone (i.e. R_{ENZYME}>R_{BLANK}) (see Table 13 and 14).

TABLE 10
Experimental setup of Mini wash assay

| | |
|---|---|
| Detergent | Liquid Model detergent A or Model detergent T |
| | (see Table 11 and 12) |
| Detergent | 3.33 g/l |
| dose | |
| pH | “as is” in the current detergent solution and |
| | was not adjusted |
| Water | 16° dH, adjusted by adding CaCl₂*2H₂O, MgCl₂*6H₂O |
| hardness | and NaHCO₃ (5:1:3) to milli-Q water. |
| Enzymes | EXc (SEQ ID NO: 17), xanthan lyase (XLb, SEQ ID NO: |
| | 22 or XLc SEQ ID NO: 23) |
| Enzyme | Dosage of GH5: 0.05 mg EP/L (enzyme protein), 0.10 mg |
| dosage | EP/L, 0.2 mg EP/L, 0.5 mg EP, 1.0 mg EP/L; experiments |
| | with combinations of GH5 and XL were conducted with a |
| | fixed concentration of 1.0 mg EP/L XL |
| Volume | 50 ml |
| of test | |
| solution | |
| Test | Xanthan Gum with carbon black DN-31D textile swatches |
| material | (23 × 3 cm). |
| | The test material was obtained from Center for |
| | Testmaterials BV, P.O. Box 120, 3133 KT Vlaardingen, |
| | the Netherlands, and WFK Testgewebe GmbH, Christenfeld |
| | 10, D-41379 Brüggen, Germany |
| Temperature | 40° C. |
| Wash time | 30 min |
| Rinse time | 5 min |
| Test | Soiled textile continuously lifted up and down into |
| system | the test solutions, 50 times per minute (up-time 0.4 sec, |
| | down-time 0.4 sec, lift time 0.4 sec). The test solutions |
| | are kept in 125 ml glass beakers. After wash of the textiles |
| | are continuously lifted up and down into tap water, 50 times |
| | per minute (up-time 0.4 sec, down-time 0.4 sec, lift time |
| | 0.4 sec). |

TABLE 11
Composition of Model Detergent A (Liquid) ¹⁾

| Detergent ingredients | Wt % |
|---|---|
| Linear alkylbenzenesulfonic acid (LAS) (Marlon AS3) | 13 |
| Sodium alkyl(C12)ether sulfate (AEOS) (STEOL CS-370 E) | 10 |
| Coco soap (Radiacid 631) | 2.75 |
| Soy soap (Edenor SJ) | 2.75 |
| Alcohol ethoxylate (AEO) (Bio-Soft N25-7) | 11 |
| Sodium hydroxide | 2 |
| Ethanol | 3 |
| Propane-1,2-diol (MPG) | 6 |
| Glycerol | 2 |
| Triethanolamine (TEA) | 3 |
| Sodium formate | 1 |
| Sodium citrate | 2 |
| Diethylenetriaminepentakis(methylenephosphonic acid) (DTMPA) | 0.2 |
| Polycarboxylate polymer (PCA) (Sokalan CP-5) | 0.2 |
| Water | Up to |
| | 100 |

¹⁾ The pH of the detergent was adjusted to pH 8 with sodium hydroxide or citric acid.

TABLE 12
Composition of Model detergent T (powder)

| Detergent ingredients | Wt % |
|---|---|
| LAS, sodium salt | 11.72 |
| AS, sodium salt | 2.0 |
| Soap, sodium salt | 2.15 |
| AEO | 3.0 |
| Soda ash | 14.98 |
| Hydrous sodium silicate | 3.12 |
| Zeolite A | 18.75 |
| HEDP-Na4 | 0.15 |
| Sodium citrate | 2.0 |
| PCA, copoly(acrylic acid/maleic acid), sodium salt | 1.65 |
| SRP | 0.5 |
| Sodium sulfate | 13.53 |
| Sodium percarbonate | 22.20 |
| TAED | 3.25 |
| Foam regulator | 1.0 |

TABLE 13
Remission (R) values obtained in Mini Wash using EXc with
and without xanthan lyase (XLb) in liquid model A detergent

| | | | EXc + |
|---|---|---|---|
| Enzyme dosage | No enzyme | EXc | xanthan lyase |
| 0.05 mg EP/L | 29.5 | 32.8 | 35.1 |
| 0.1 mg EP/L | 29.5 | 33.6 | 35.4 |
| 0.2 mg EP/L | 29.5 | 34.3 | 35.9 |
| 0.5 mg EP/L | 29.5 | 35.1 | 36.7 |
| 1.0 mg EP/L | 29.5 | 35.4 | 37.3 |

TABLE 14
Remission (R) values obtained in Mini Wash using EXc with
and without Xanthan Lyase (XLc)in powder model T detergent

| | | | EXc + |
|---|---|---|---|
| Enzyme dosage | No enzyme | EXc | xanthan lyase |
| 0.05 mg EP/L | 29.8 | 29.7 | 29.7 |
| 0.1 mg EP/L | 29.8 | 29.8 | 29.8 |
| 0.2 mg EP/L | 29.8 | 30.0 | 30.0 |
| 0.5 mg EP/L | 29.8 | 30.6 | 30.9 |
| 1.0 mg EP/L | 29.8 | 31.0 | 31.2 |

### Example 10: Wash Performance of Combinations of a GH5 Polypeptide and Xanthan Lyase was Tested on Specific Stains

The wash performance of variants in liquid and powder detergents was determined by using the following standardized stains, all obtainable from CFT (Center for Testmaterials) B.V., Vlaardingen, Netherlands:

A: Fluid make-up: product no. PCS17

B: Fluid make-up: product no. CS17

For the tests in liquid detergents, a liquid washing agent with the following composition was used as base formulation (all values in weight percent): 0 to 0.5% xanthan gum, 0.2 to 0.4% antifoaming agent, 6 to 7% glycerol, 0.3 to 0.5% ethanol, 0 to 7% FAEOS (fatty alcohol ether sulfate), 10 to 28% nonionic surfactants, 0.5-1% boric acid, 1 to 2% sodium citrate (dihydrate), 2 to 4% soda, 0 to 16% coconut fatty acid, 0.5% HEDP (1-hydroxyethane-(1,1-diphosphonic acid)), 0 to 0.4% PVP (polyvinylpyrrolidone), 0 to 0.05% optical brighteners, 0 to 0.001% dye, remainder deionized water.

Based on this base formulation, detergent was prepared by adding the respective enzyme combination as indicated in table 15. As a reference, the detergent composition without addition of the enzyme combinations was used.

The dosing ratio of the liquid washing agent was 4.7 grams per liter of washing liquor and the washing procedure was performed for 60 minutes at a temperature of 40° C., the water having a water hardness between 15.5 and 16.5° (German degrees of hardness).

For the tests in solid detergents, a European premium detergent was used as base formulation.

The whiteness, i.e. the brightening of the stains, was determined photometrically as an indication of wash performance. A Minolta CM508d spectrometer device was used, which was calibrated beforehand using a white standard provided with the unit.

The results obtained are the difference values between the remission units obtained with the detergents and the remission units obtained with the detergent containing the enzyme combinations. A positive value therefore indicates an improved wash performance due to the enzyme combinations present in the detergent. It is evident from table 15 that enzyme combinations according to the invention show improved wash performance.

TABLE 15
Wash performance in liquid detergent

| Enzyme combination | | A | B |
|---|---|---|---|
| XLb SEQ ID NO: 22 + EXc SEQ ID NO: 17 | Diff | 3.3 | 6.4 |
| | HSD | 2.4 | 1.2 |

TABLE 16
Wash performance in solid detergent

| Enzyme combination | | B |
|---|---|---|
| XLb SEQ ID NO: 22 + EXc SEQ ID NO: 17 | Diff | 1.9 |
| | HSD | 1.2 |

### Example 11: Wash Performance of GH5 Polypeptides with and without Xanthan Lyase

In this example wash performance of GH5 polypeptides was evaluated in a liquid model detergent A washed in the Automatic Mechanical Stress Assay (AMSA) at 20° C. or 40° C. The wash performance of the enzymes was evaluated either alone or in combination with a Xanthan Lyase. The wash conditions used are specified in Table 17 below.

TABLE 17
Wash conditions used in the example 11:

| | |
|---|---|
| Detergent | Liquid model detergent A |
| Detergent | 3.3 g/L |
| conc. | |
| pH | “as is” in the current detergent |
| | solution and was not adjusted |
| Temperature | 20° C. or 40° C. |
| Dosages in | 140 μL detergent per slot; 20 μL enzyme per slot |
| AMSA-plate | |
| Water | 16° dH, adjusted by adding CaCl₂*2H₂O, MgCl₂*6H₂O |
| hardness | and NaHCO₃ (5:1:3) to milli-Q water |
| Enzymes | EXb (SEQ ID NO: 16); EXc (SEQ ID NO: 17), |
| | xanthan lyase (XLb, SEQ ID NO: 22) |
| Enzyme | EXb and EXc concentrations: 0.7, 1.5, 20, 125 ppb |
| dosage | XLb concentration: 400 ppb |
| Test solution | 160 micro L |
| volume | |
| Wash time | 20 minutes |
| Stain/swatch | Mayonaise with carbon black C-S-05 S from CFT, |
| | Center for Testmaterials BV. |

The enzyme and wash liquid were dosed into the AMSA plate and washed according to conditions listed in Table 17. After wash the fabric was flushed in tap water and air-dried. The performance of the enzyme was subsequently measured as the brightness of the colour of the textile samples. Brightness was measured as the intensity of the light reflected from the textile sample when illuminated with white light. Intensity was measured with a professional flatbed scanner EPSON EXPRESSION 10000XL with special designed software that extracted the intensity value from the scanned imagine through standard vector calculations.

The performance of the enzyme (or combination of enzymes) was compared to the performance of detergent alone (blank) or detergent with the Xanthan lyase (XL). An enzyme (or combination of enzymes) was considered to exhibit improved wash performance if it performed better than the detergent alone (i.e., R_{ENZYME}>R_{BLANK}) (see Tables 18, 19, 20 and 21).

TABLE 18
Intensity and delta intensity of GH5 polypeptides
EXb (SEQ ID NO: 16) and EXc (SEQ ID NO: 17) tested
in AMSA at 20° C. in model detergent A.

| | Intensity | | | | Delta intensity | | | |
|---|---|---|---|---|---|---|---|---|
| | Concentration [ppb] | | | | | | | |
| | 0.7 | 1.5 | 20 | 125 | 0.7 | 1.5 | 20 | 125 |
| Blank | 210.4 | 210.4 | 210.4 | 210.4 | | | | |
| EXb (SEQ ID | 210.8 | 212.8 | 217.2 | 217.8 | 0.4 | 2.4 | 6.8 | 7.5 |
| NO: 16) | | | | | | | | |
| EXc (SEQ ID | 212.0 | 214.4 | 216.5 | 218.4 | 1.6 | 4.1 | 6.2 | 8.0 |
| NO: 17) | | | | | | | | |

TABLE 19
Intensity and delta intensity of GH5 polypeptides
EXb (SEQ ID NO: 16) and EXc (SEQ ID NO: 17) tested
in AMSA at 40° C. in model detergent A.

| | Intensity | | | | Delta intensity | | | |
|---|---|---|---|---|---|---|---|---|
| | Concentration [ppb] | | | | | | | |
| | 0.7 | 1.5 | 20 | 125 | 0.7 | 1.5 | 20 | 125 |
| Blank | 220.0 | 220.0 | 220.0 | 220.0 | | | | |
| EXb (SEQ ID | 221.9 | 222.9 | 229.4 | 230.2 | 1.9 | 3.0 | 9.4 | 10.2 |
| NO: 16) | | | | | | | | |
| EXc (SEQ ID | 223.2 | 225.4 | 228.3 | 229.0 | 3.3 | 5.4 | 8.3 | 9.0 |
| NO: 17) | | | | | | | | |

TABLE 20
Intensity and delta intensity of GH5 polypeptides EXb (SEQ ID
NO: 16) and EXc (SEQ ID NO: 17) with Xanthan lyase (XLb (SEQ
ID NO: 22) tested in AMSA at 20° C. in model detergent A.

| | Intensity | | | | Delta intensity | | | |
|---|---|---|---|---|---|---|---|---|
| | Concentration [ppb] | | | | | | | |
| | 0.7 | 1.5 | 20 | 125 | 0.7 | 1.5 | 20 | 125 |
| Blank with XLb | 214.0 | 214.0 | 214.0 | 214.0 | | | | |
| (SEQ ID NO: 22) | | | | | | | | |
| EXb (SEQ ID | 213.0 | 215.3 | 220.4 | 223.7 | −1.0 | 1.3 | 6.4 | 9.7 |
| NO: 16 with XLb | | | | | | | | |
| (SEQ ID NO: 22) | | | | | | | | |
| EXc (SEQ ID | 212.4 | 215.1 | 220.2 | 221.4 | −1.6 | 1.1 | 6.2 | 7.4 |
| NO: 17) with XLb | | | | | | | | |
| (SEQ ID NO: 22) | | | | | | | | |

TABLE 21
Intensity and delta intensity of GH5 polypeptides EXb (SEQ ID
NO: 16) and EXc (SEQ ID NO: 17) with Xanthan lyase (XLb (SEQ
ID NO: 22) tested in AMSA at 40° C. in model detergent A.

| | Intensity | | | | Delta intensity | | | |
|---|---|---|---|---|---|---|---|---|
| | Concentration [ppb] | | | | | | | |
| | 0.7 | 1.5 | 20 | 125 | 0.7 | 1.5 | 20 | 125 |
| Blank with XLb | 220.6 | 220.6 | 220.6 | 220.6 | | | | |
| (SEQ ID NO: 22) | | | | | | | | |
| EXb (SEQ ID | 222.0 | 225.0 | 231.0 | 232.6 | 1.3 | 4.4 | 10.3 | 12.0 |
| NO: 16 with XLb | | | | | | | | |
| (SEQ ID NO: 22) | | | | | | | | |
| EXc (SEQ ID | 222.3 | 223.9 | 230.1 | 231.5 | 1.7 | 3.2 | 9.5 | 10.9 |
| NO: 17) with XLb | | | | | | | | |
| (SEQ ID NO: 22) | | | | | | | | |

The results in above tables show that the GH5 polypeptides, e.g., EXb and EXc, have an improved wash performance both when evaluated alone or in combination with the Xanthan Lyase, e.g., XLb.

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

1. A polypeptide of glycosyl hydrolase family 5 having xanthan degrading activity.

2. The polypeptide of claim 1, selected from the group consisting of:
(a) a polypeptide having at least 60% sequence identity to the mature polypeptide of SEQ ID NO: 2;
(b) a polypeptide encoded by a polynucleotide that hybridizes under medium stringency conditions with (i) the mature polypeptide coding sequence of SEQ ID NO: 1, (ii), or the full-length complement of (i);
(c) a polypeptide encoded by a polynucleotide having at least 60% sequence identity to the mature polypeptide coding sequence of SEQ ID NO: 1;
(d) a variant of the mature polypeptide of SEQ ID NO: 2, comprising a substitution, deletion, or insertion at one or more positions;
(e) a fragment of the polypeptide of (a), (b), (c), or (d) that has xanthan degrading activity; and
(f) a polypeptide comprising the polypeptide of (a), (b), (c), (d), or (e) and a N-terminal or C-terminal His-tag.

3. The polypeptide of claim 1, selected from the group consisting of:
(a) a polypeptide having at least 60% sequence identity to the mature polypeptide of SEQ ID NO: 4;
(b) a polypeptide encoded by a polynucleotide that hybridizes under medium stringency conditions with (i) the mature polypeptide coding sequence of SEQ ID NO: 3, (ii), or the full-length complement of (i);
(c) a polypeptide encoded by a polynucleotide having at least 60% sequence identity to the mature polypeptide coding sequence of SEQ ID NO: 3;
(d) a variant of the mature polypeptide of SEQ ID NO: 4, comprising a substitution, deletion, or insertion at one or more positions;
(e) a fragment of the polypeptide of (a), (b), (c), or (d) that has xanthan degrading activity; and
(f) a polypeptide comprising the polypeptide of (a), (b), (c), (d), or (e) and a N-terminal/or C-terminal His-tag.

4. The polypeptide of claim 1, selected from the group consisting of:
(a) a polypeptide having at least 60% sequence identity to the mature polypeptide of SEQ ID NO: 6;
(b) a polypeptide encoded by a polynucleotide that hybridizes under medium stringency conditions with (i) the mature polypeptide coding sequence of SEQ ID NO: 5, (ii), or the full-length complement of (i);
(c) a polypeptide encoded by a polynucleotide having at least 60%, sequence identity to the mature polypeptide coding sequence of SEQ ID NO: 5;
(d) a variant of the mature polypeptide of SEQ ID NO: 6, comprising a substitution, deletion, or insertion at one or more positions;
(e) a fragment of the polypeptide of (a), (b), (c), or (d) that has xanthan degrading activity; and
(f) a polypeptide comprising the polypeptide of (a), (b), (c), (d), or (e) and a N-terminal or C-terminal His-tag.

5. The polypeptide of claim 1, selected from the group consisting of:
(a) a polypeptide having at least 60% sequence identity to the mature polypeptide of SEQ ID NO: 8;
(b) a polypeptide encoded by a polynucleotide that hybridizes under medium stringency conditions with (i) the mature polypeptide coding sequence of SEQ ID NO: 7, (ii), or the full-length complement of (i);
(c) a polypeptide encoded by a polynucleotide having at least 60% sequence identity to the mature polypeptide coding sequence of SEQ ID NO: 7;
(d) a variant of the mature polypeptide of SEQ ID NO: 8, comprising a substitution, deletion, or insertion at one or more positions;
(e) a fragment of the polypeptide of (a), (b), (c), or (d) that has xanthan degrading activity; and
(f) a polypeptide comprising the polypeptide of (a), (b), (c), (d), or (e) and a N-terminal or C-terminal His-tag.

6. The polypeptide of claim 2, having at least 65% sequence identity to the mature polypeptide of SEQ ID NO: 2.

7. The polypeptide of claim 2, which is encoded by a polynucleotide that hybridizes under medium-high stringency conditions with (i) the mature polypeptide coding sequence of SEQ ID NO: 1, or (ii) the full-length complement of (i).

8. The polypeptide of claim 2, which is encoded by a polynucleotide having at least 65% sequence identity to the mature polypeptide coding sequence of SEQ ID NO: 1.

9. The polypeptide of claim 2, consisting of SEQ ID NO: 2, or the mature polypeptide of SEQ ID NO: 2.

10. The polypeptide of claim 2, comprising SEQ ID NO: 2, or the mature polypeptide of SEQ ID NO: 2.

11. The polypeptide of claim 2, which is a variant of the mature polypeptide of SEQ ID NO: 2, comprising a substitution, deletion, or insertion at up to 10 positions.

12. The polypeptide of claim 2, which is a fragment of SEQ ID NO: 2, wherein the fragment has xanthan degrading activity.

13. A polynucleotide encoding the polypeptide of claim 2.

14. A nucleic acid construct or expression vector comprising the polynucleotide of claim 13 operably linked to one or more control sequences that direct the production of the polypeptide in an expression host.

15. A recombinant host cell comprising the polynucleotide of claim 13 operably linked to one or more control sequences that direct the production of the polypeptide.


SEQUENCE LISTING (24 sequences)

SEQ ID NO: 1 - DNA, 2517 bp, Opitutaceae sp
  CDS (1)..(2514)
  sig_peptide (1)..(108)
  mat_peptide (109)..(2514)
atgcaatcatcaagctcaaattcggtcgtatccgcttcccggatactccgacgcttctccctcccgctgctcgccgccgcgctgggcctcgccgcgcccgcccgcgccgccgactattacctgaaggccagccaaggcgcatccaaccactggtcctcccatctcaccgactggaccgccaacgccgacggcaccggcgccaacccgacggtcatcggcctggccgacaccttcgacaccaacaaccgcacgcttcgcactcccgccgtcaacgccaccaccacctacccgggcggcgtgctccgcctttccggcggcgccggcgtcatcggcatgaagactggcggcaccgccgtcgccatcgtgcccaagctcgtctccaccgccggcaccgtggacgcctggcacaccggcacccaatacttccgcgccgacgactgggagaacctcgcctccggcaccgggttcaccgcgctcaaggccgtcgccggccgcacgctcaaggtcagcgtcggcaagctcaccggctccggcgagacccgtcttcacggcggcggcgccgtccgcctcgacgtcaccgacggcgaacgctacctcggcgtcgtccgcgtctcctccggcgcggccgacttcgacaacaacgtgttcgtctccggcccgctcgtgatcgagaccggcgcgaccgtcgtgctcgaccaggccgtctccttcgccggcctgaccgtcgccggcaccgagtattcgcccggcaactacaccttcgccgcgctccaggccgcgcatcctacggtgttcacctccggcaccgccggcggctcgatcaccgtccgcgccccgcgcacctggtatctcaccgtgaatcagggcggcgtgcagaactggaccgagacctacctttcgaactggaactccgccgccaatggctccggcgtcgcgccgacttcgatcaacggctacgacttctacatcgatcaggtctccaaccgcgagatccgcacgccctccaccgcctccaccttcggcggcggcgcgctcgccctcgccagcggcgccaagctcaccctcaagagttcgcccggcgtcgtcagcaccatcccggcgttcgtgaacacgaactccccgatcatcgtgaacggcggcggtagcttccgccaaagtctcgccctcggtgactgggagatcgcctccggcatcaccaagctctccgccggctccggtcgcagcctcggcttcgacatcgactacctcggcggcgcgggtggccttgtcacccaaaacggcggctcttacttcctcagcctcgacgacggctccggctacaccggcacgctcaaccacgcgtccggcgcgctccgcttcgagtccgtcttctccaccgagggcgcgctcaccatcggctcctcggcgaccgtccacctcgaccaacaggtttacgtcacgtcgttctccgtcgccggtgtcgccaaggccgccggcatccacacctacgcctcgctgaacgccgcgcatcccgcacagttcaccgccggcgccgcgcccggactcgtcgctgtttacacgcccgacaccgccggccccgtccgcatgaacggcgtcaatatctccggccccgagagcaacaccgccaacctccccggcacctacggctacaactacgtttaccccaccgaggccgacttcgactactacgcctccaagggcctcaacctcatccgcattcccttccgctgggagcgcatgcagcacggcctgaacgttccgctcaacaccgcccagctcggctacatggacaccgccgtcgcccgcgcctccgcgcgcggcatgaaggtcatcctcgatatgcacaactacgcccgctgcaaagtcggcggagtcacctacaagttcggcgacgcgcagctccccgcctcggcctacgccgacgtctggcgccgtctcgccgaccactacaaaaacgagcccgccatctacggcttcgacatcatgaacgagcccaacggcctctccggcggcgtctggcccgcctacgcccaggccgcggtcaacgccatccgcgaggtcaatctgtccacctgggtcatcgtcgagggcgagttttgggccaacgcttggggcttcgagaccaagaacccgtatctgcacaacgtccgcgatcccgtcggccgcctcatgttctccgcccactcctactggagcgacgccggcaccgatgtttacaagacctacgacgaagagggcgcctatcccgagatgggcgtgaacaacgtgaagcccttcatcgactggctgaagaagcacgacgccaagggcttcgtcggcgaatacggcgtgcccaacaacgacccgcgctggctcgtcgtgctggacaacttcctcgcctacctcgcggccgagggcgtgagcggcacctactgggccggcggcgcctggtattcgggcagcccgatcagctgccacccgtcctccaactacaccgtggatcgcgccgtcatgagcgtgctcgaagaccatccatga

SEQ ID NO: 2 - protein, 838 aa, Opitutaceae sp
MQSSSSNSVVSASRILRRFSLPLLAAALGLAAPARAADYYLKASQGASNHWSSHLTDWTANADGTGANPTVIGLADTFDTNNRTLRTPAVNATTTYPGGVLRLSGGAGVIGMKTGGTAVAIVPKLVSTAGTVDAWHTGTQYFRADDWENLASGTGFTALKAVAGRTLKVSVGKLTGSGETRLHGGGAVRLDVTDGERYLGVVRVSSGAADFDNNVFVSGPLVIETGATVVLDQAVSFAGLTVAGTEYSPGNYTFAALQAAHPTVFTSGTAGGSITVRAPRTWYLTVNQGGVQNWTETYLSNWNSAANGSGVAPTSINGYDFYIDQVSNREIRTPSTASTFGGGALALASGAKLTLKSSPGVVSTIPAFVNTNSPIIVNGGGSFRQSLALGDWEIASGITKLSAGSGRSLGFDIDYLGGAGGLVTQNGGSYFLSLDDGSGYTGTLNHASGALRFESVFSTEGALTIGSSATVHLDQQVYVTSFSVAGVAKAAGIHTYASLNAAHPAQFTAGAAPGLVAVYTPDTAGPVRMNGVNISGPESNTANLPGTYGYNYVYPTEADFDYYASKGLNLIRIPFRWERMQHGLNVPLNTAQLGYMDTAVARASARGMKVILDMHNYARCKVGGVTYKFGDAQLPASAYADVWRRLADHYKNEPAIYGFDIMNEPNGLSGGVWPAYAQAAVNAIREVNLSTWVIVEGEFWANAWGFETKNPYLHNVRDPVGRLMFSAHSYWSDAGTDVYKTYDEEGAYPEMGVNNVKPFIDWLKKHDAKGFVGEYGVPNNDPRWLVVLDNFLAYLAAEGVSGTYWAGGAWYSGSPISCHPSSNYTVDRAVMSVLEDHP

SEQ ID NO: 3 - DNA, 2496 bp, Unknown
  Environmental sample
  CDS (1)..(2493)
  sig_peptide (1)..(111)
  mat_peptide (112)..(2493)
atgaacaccacaccacaacccacccccgcccgccggacgcctcgacgcccgttcctcgccaccctcgctaccatcctcggcctcgccgcctccgtctcctccgtctccgccgccgactggtatctcgataaaaaccaggcccgctacgccagctgggacaccctcgccgactggaaacccaaccccgacggcagcggctccaacccctccgccctctccccctccgacacctaccacctcaacggcttcatgctccgcacccccgagggcggctccacctacaccttcaccggcggcctcctcagcctcgccaacaacgccgacaacttcgccctcaagaccaccggctccggcgtctccatcatccccgccctgcgcaccaccgccggcctcgtccaaaacgtcggctccggcacgcaaaacctccaggttggccactaccaaaacctctccggcacgacctcctactacgcccagaccgggcgcggcctcaacctcgccatcaccaccctcgtgggctccggccagttccgcttctacggcggcggcacctactacctctccctcgccaactccccgacctacgacggcgacatctacgtccaatccggcaccatcgatttcaacaacgacctcgccaccgccggcactctcaccgtcaacaccggtgccaaggtcgccctcgaccaggccgtcaccttcaccggcctcaccatagccggcacagcgtatccagttggaaactacagctacgccgcgcttcaggccgcccaccccgccgttttcgtctccggcacctccggcggagccatcaacgtccgcgccccgcgcaactggtatctctccacccaccaacccgtcggcgccagctggaacaccctcgcccattggcgcgccaaccccgacggcaccggcgccaccgccgactccatcaactccttcgacaactacatcaaccaagtctccggccgcaccctgcgcacccccgaaaccaccgccaccttcgccggcggttccctcgtcctcgccgacggcggcaacctctcgctcaaggcccccgccggccactccagcaccatccccgccttcgccacatcgggatcgatttccatcaccaacggcttcagcagcatcacccagcccctcgtcatcggcgactggcacctcggcgccggcaccgcccaagtctccgtgccaagcaccagcaccgtgcagctcaccgtcgataaactctccggcgacggcaccctccagttccagaacggcggcaaatacaccctcaacatccgcggcgcgtccgccttcaccggcaccctccgccacctctccggcacgctcaccgtagcctcccagatcggcaccggcggcaccctcgtcgtcgaatccaccggcgcggtgaaactcgaccaccccggcttcttcaccggcgtcaccgtcgccggcacgcccctcgcccccggctaccacacctacgccgcgctcaaagccgcccaccccgcgcgcttccccaccggctccaccaacgccttcctcgccgtctatccgcccgacaccaccggccccgcccacatgttcggcgtcaacctcgccggcggcgaattcggcaccccgatgcccggcgtttacggcaccgactacatctacccgagcgccgccgccttcgattactaccacggcaaaggcctcaaactcatccgcctcccctttaagtgggaacgcctccagcacaccctcaacgcccccctcaacgccgccgagctcgcccgcatcgacaccgtcgtcggctacgcctccgcgcgcggcatgaaggtcgtcctcgacatgcacaactacgcccgccgcaaagaaagcggcaccacctacctcatcggcaccggccccgtcaccatggacgccttcggcgacgtctggcgtcgcatcgccgatcactacaagggcaaccccgccatctacggctacggcatcatgaacgagccctactccaccaacaccacctggccccagatggcccagaccgccgtcaacgccatccgcaccgttgacctcaccacccacgtcatcgtcgccggcgacggctggtccaacgccaccggctggcgctccaagaaccccaacctcgacacccaggaccccgtcggccgcctcatctacgaagcccactgctacttcgattccaacctctccggcacctacacccaaagctacgatgccgccggcgcccaccccatgatcggcgtggaccgcgtgcgcgaattcgtcgagtggcttcaggaaaccggcaacaaaggcttcatcggcgaatacggcgtccccggcaacgacccccgctggctcgtcgtgctcgacaacttcctcgcctacctcgacgccaacggcgtctccggcacctactgggccggcggtccttggtggggcaactacccgctcagctgcgaacccacctccaactacaccgtggacaaaccccagatgagcgtcctcgaaaactacaactga

SEQ ID NO: 4 - protein, 831 aa, Unknown
  Synthetic Construct
MNTTPQPTPARRTPRRPFLATLATILGLAASVSSVSAADWYLDKNQARYASWDTLADWKPNPDGSGSNPSALSPSDTYHLNGFMLRTPEGGSTYTFTGGLLSLANNADNFALKTTGSGVSIIPALRTTAGLVQNVGSGTQNLQVGHYQNLSGTTSYYAQTGRGLNLAITTLVGSGQFRFYGGGTYYLSLANSPTYDGDIYVQSGTIDFNNDLATAGTLTVNTGAKVALDQAVTFTGLTIAGTAYPVGNYSYAALQAAHPAVFVSGTSGGAINVRAPRNWYLSTHQPVGASWNTLAHWRANPDGTGATADSINSFDNYINQVSGRTLRTPETTATFAGGSLVLADGGNLSLKAPAGHSSTIPAFATSGSISITNGFSSITQPLVIGDWHLGAGTAQVSVPSTSTVQLTVDKLSGDGTLQFQNGGKYTLNIRGASAFTGTLRHLSGTLTVASQIGTGGTLVVESTGAVKLDHPGFFTGVTVAGTPLAPGYHTYAALKAAHPARFPTGSTNAFLAVYPPDTTGPAHMFGVNLAGGEFGTPMPGVYGTDYIYPSAAAFDYYHGKGLKLIRLPFKWERLQHTLNAPLNAAELARIDTVVGYASARGMKVVLDMHNYARRKESGTTYLIGTGPVTMDAFGDVWRRIADHYKGNPAIYGYGIMNEPYSTNTTWPQMAQTAVNAIRTVDLTTHVIVAGDGWSNATGWRSKNPNLDTQDPVGRLIYEAHCYFDSNLSGTYTQSYDAAGAHPMIGVDRVREFVEWLQETGNKGFIGEYGVPGNDPRWLVVLDNFLAYLDANGVSGTYWAGGPWWGNYPLSCEPTSNYTVDKPQMSVLENYN

SEQ ID NO: 5 - DNA, 2508 bp, Unknown
  Environmental sample
  CDS (1)..(2505)
  sig_peptide (1)..(105)
  mat_peptide (106)..(2505)
atgaaacaccaccacaccacaccacacaccccgcgtcggaccctgctccgctcgcttgccggcctgctggctctcgccaccggcctcgcctccaccgcccacgccgccgactactacctcaaagtcaaccaaccccaccccaacagctgggcctcacccgtcaccgattgggccgccaaccccgacggcaccggagccgctcccgccgccatcgccgcgcccgacaccttttacaccaacaaccgcacgctccgcacccccgccgtcggcgtcaacgccaccttccccggcggcgtcctcggcctaaacggcggcgtcatcggcataaaaaccggcccctccgccttctccatcgcccccaagctcgtctccaccgccggcgccatcgagtcctggggcacaccccaaaacttccgcgccgacgactgggagagcaacgcccccttccccaccttcaccggactgaggaccgcctccaaccatacgctcaaggtctccgtcggcaaactctccggcaccggcgaaatccgcgtccacggcggcggcaccgtcctcctcgacgtcaccgacgccgaaaactacctcggcaccctctgcgtcgcctccggcgcgttgaacttcgacaacgccgtcttctcctccggccccctcgacatcaagaccggcgccaccgtcgtcctcgaccaggccgtctccttcgccggcctcgccgtcggagccaccgagtatccacccggcaactacaccctcgccgccctgcaagccgcccacccgggcgtcttcaccggcaccgccgccggctccatcaccgtccgcgccccgcgcacctggtatctcaccgtcagccagggctcccagaactggaccgaggccttcctctccaactggaactccgccgccaacggctccggcgtcgccccgaactacatcaacggccacgacatctacctcaaccaggtgaacaaccgcgagctccgcacgccctacaccgccagcaccttcaccggcggcaccctcgccctcaccttcggctcgaagctcgtcgtcaagacctcacccaacctcgtcagcaccatccccgccctcgtcacctccggcaccccgcagttcgccaacggcagcggcagccgccaaaacctcgccatcggcgactgggacatcatctccggcaccagccgcctcgtcgccggctccacccggtccctcggcttcgacatcggctggctcaccggcgcgggcaacctccagaccgaaggcggcggctcgttcttcctccgcctcatcgacggctccggctacaccggcgccatcaaccacaactccggcgccctccgcttcgagtccgtcttctccaccgccggtgccctcaacatcggcgcctccgcgaccgtccacctcgacaagcccgtctatgtcagcggcctctccgtcgccggcgtcgccaaacccgccggcatccacacctacgcctcgctgaacgccgcgcatcccgcgcagttcaacgccggcgccgcgcccggactcgtcgccgtttacacacccaacactgccgcccccgtccgcatgaacggcgtcaacctctccggccccgaatccgtcggcggcgccggcacgccctttcccggcacctacggcttccagtggatttaccccaccgtcgccgactacgactactacgccgccaagggccttaacctcatccgcatcccattccgctgggaacgcatgcaaggcacccttaacggtcccctcatcgccgccgaactcgctcgcatggacaacgccatcgccctcgcctccgcgcgcggcatgaaggtcatcctcgatatgcataactacgcgcgctaccgcaccccgaccgcgagctacgtgtttggtgacgcccagctccccgcctccgccttcgccgacgtctggcgcaagctcgccgatcactacaaaaacgaacccgccatctacggtttcgacatcatgaacgagccgcacagcatgcccacccccaccacctggcccacctacgcccaagccgccgtccacgccatccgcgaggtcaacctcgacacctggatcatcgtagagggcgagacctatgccaactcctggaaattcggggaaaaaaatccccacctccacaacgtgcgcgaccccgtcggccgcctcatgttctccgcccactcctactggtgcaaaaacggcgacgacagatacggcacctacgacgcggaaaacggccacccccagatgggcgtggacagcctcaagcacttcgttgactggctccgcaaacacaacgcccacggcttcgtcggcgaatacggcgtccccaacaacgacccccgctggctcgaagtccttgaaaacgcgctcatctacctggcgaatgaaaacatcagcggcacctactgggccggcggcgcctggctcgccggcagccacatcagctgccacccgtcctccaactacaccgtggaccgccccgtcatgagcgtcctccaaaactacccgtaa

SEQ ID NO: 6 - protein, 835 aa, Unknown
  Synthetic Construct
MKHHHTTPHTPRRTLLRSLAGLLALATGLASTAHAADYYLKVNQPHPNSWASPVTDWAANPDGTGAAPAAIAAPDTFYTNNRTLRTPAVGVNATFPGGVLGLNGGVIGIKTGPSAFSIAPKLVSTAGAIESWGTPQNFRADDWESNAPFPTFTGLRTASNHTLKVSVGKLSGTGEIRVHGGGTVLLDVTDAENYLGTLCVASGALNFDNAVFSSGPLDIKTGATVVLDQAVSFAGLAVGATEYPPGNYTLAALQAAHPGVFTGTAAGSITVRAPRTWYLTVSQGSQNWTEAFLSNWNSAANGSGVAPNYINGHDIYLNQVNNRELRTPYTASTFTGGTLALTFGSKLVVKTSPNLVSTIPALVTSGTPQFANGSGSRQNLAIGDWDIISGTSRLVAGSTRSLGFDIGWLTGAGNLQTEGGGSFFLRLIDGSGYTGAINHNSGALRFESVFSTAGALNIGASATVHLDKPVYVSGLSVAGVAKPAGIHTYASLNAAHPAQFNAGAAPGLVAVYTPNTAAPVRMNGVNLSGPESVGGAGTPFPGTYGFQWIYPTVADYDYYAAKGLNLIRIPFRWERMQGTLNGPLIAAELARMDNAIALASARGMKVILDMHNYARYRTPTASYVFGDAQLPASAFADVWRKLADHYKNEPAIYGFDIMNEPHSMPTPTTWPTYAQAAVHAIREVNLDTWIIVEGETYANSWKFGEKNPHLHNVRDPVGRLMFSAHSYWCKNGDDRYGTYDAENGHPQMGVDSLKHFVDWLRKHNAHGFVGEYGVPNNDPRWLEVLENALIYLANENISGTYWAGGAWLAGSHISCHPSSNYTVDRPVMSVLQNYP

SEQ ID NO: 7 - DNA, 2082 bp, Pseudomonas stutzeri
  CDS (1)..(2079)
  sig_peptide (1)..(108)
  mat_peptide (109)..(2079)
atgtccaccaacctgttttccggtgcccgcaaggcactcgtcgcttccatcgctgccgctgttctgctgggtggcgccactgttgtaaccacgccttatgccgctgcatcctcggttgccgctgtatcggtttccgccaagatcaacgcgttcaccaacagcgattggctgaacggtatctggcgcaccggcgccggcttctcgatccccgccacctccgcaaaccgcgccgcgttcgtggccggcgcttcggtacgactggcagacggtcaggtacgcaagatcagccgcgcgcaaatcgtcggcagcaacatgagcatcttcctggaaggtgcaaagctggacggcaacaaggttggcgcaccgcaagtggtcaccatcggcagcacggccgtaacggccccggacacttctgctccgatcactacaccgcctaccgttactgcgcactcgaccagcatcaacgcattcaccaacaatgattggctcaatggtgtatggcgtaagtcgccgggcttctccattccggcaagcgctgccaacaaggctgctttcaaagttggagcgacagcaaaactggcagatggccaggttcgcaaaattacccaggtacaagttgttggcgccaatatgagcgtctatctggaaggtgcggcagttaacggaagtgtcgtcggcgcacccaacaagttggcgctggctacaacttcgactaccagcccggctccgactccggcgcccagtgctccgaccccttcggtcatcgccaccagcaacctgaacaactacaccaatgctcaatggctcaacggtatgtaccgtaccgctgcaggcttctccatccaggcaagcagcgccaacgtggcggcattcaaggctggcgctttggtgaggctcgctgatggtcagacccgcaaggtgctgcgcgctcagctggtcggcagcaacatgagcgtctttcttgacggcgcggtaatcaacggtacgaccctgggctatccgaagaccatctcggtggtcagtacgtcgaccggcactccttcgtctcctgctctgactaccccaccggtagagccagcaccggctccggtgcccaccgcacctgacaccaccaatggcaagccgctgctggttggcgtcaatctgtccggcgccggcttcggtccctcggttgttcccggcaagcatggcaccaactacacctatcctgccgagtcgtactacaagaagtattccgacctgggcatgccgctggttcgcctgccgttcctctgggagcgtatccagcccaagctgaactctccgctgaacgccgaggagttcgcccgtctgaagcagtcgctggatttcgcgcagaagcacaacgtcaaggtgattctcgacctgcacaactactaccgttattacggcaagctgatcggctccaaagaagtgcccatcagttccttcgccgcggtatggaagcagatcgtgcagcaagtagtgaaccacccggccgtcgaaggctacggcctgatgaacgagccgcactcgaccaacgggctctggccgcaggctgccctggcggctgctcaggcaatccgcaccgtcgactccaagcgctggatctacgtagcaggcgatcgctggtcgagcgctttccactggccgcactacaacactcagctggtcaccaacccgtggatgcgcgatccgaagaacaatctggtttacgaagcgcacatgtacgtggacaaggatttctcgggcaactacttcgacaaggccgagaagttcgacccgatgattggcgtcaaccgcgtcaagcccttcgtcgactggctcaagcagcacaaactgcgcggctacatcggtgagcacggcgtaccggatttctcgccctcggccatcgtcgcaaccgataacctgctggcctacctgcgtcagaactgcatcccgagcacctattgggctgccggtccctggtggggcgagtacgcgatgtccctggacgtaagcagcggcaagcaccgtccgcagctgccggttctgcagaagcacgccaaaaccgcaaacagctgcaccagcatcggtccgctgtaa

SEQ ID NO: 8 - protein, 693 aa, Pseudomonas stutzeri
MSTNLFSGARKALVASIAAAVLLGGATVVTTPYAAASSVAAVSVSAKINAFTNSDWLNGIWRTGAGFSIPATSANRAAFVAGASVRLADGQVRKISRAQIVGSNMSIFLEGAKLDGNKVGAPQVVTIGSTAVTAPDTSAPITTPPTVTAHSTSINAFTNNDWLNGVWRKSPGFSIPASAANKAAFKVGATAKLADGQVRKITQVQVVGANMSVYLEGAAVNGSVVGAPNKLALATTSTTSPAPTPAPSAPTPSVIATSNLNNYTNAQWLNGMYRTAAGFSIQASSANVAAFKAGALVRLADGQTRKVLRAQLVGSNMSVFLDGAVINGTTLGYPKTISVVSTSTGTPSSPALTTPPVEPAPAPVPTAPDTTNGKPLLVGVNLSGAGFGPSVVPGKHGTNYTYPAESYYKKYSDLGMPLVRLPFLWERIQPKLNSPLNAEEFARLKQSLDFAQKHNVKVILDLHNYYRYYGKLIGSKEVPISSFAAVWKQIVQQVVNHPAVEGYGLMNEPHSTNGLWPQAALAAAQAIRTVDSKRWIYVAGDRWSSAFHWPHYNTQLVTNPWMRDPKNNLVYEAHMYVDKDFSGNYFDKAEKFDPMIGVNRVKPFVDWLKQHKLRGYIGEHGVPDFSPSAIVATDNLLAYLRQNCIPSTYWAAGPWWGEYAMSLDVSSGKHRPQLPVLQKHAKTANSCTSIGPL

SEQ ID NO: 9 - DNA, 2409 bp, Artificial
  Codon optimized
gcagactattatctgaaagcatcacaaggcgcatcaaatcattggtcatcacatctgacagattggacagcaaatgcagatggcacaggcgcaaatccgacagttattggcctggcagatacatttgatacaaataatcgcacactgagaacaccggcagttaatgcaacaacaacatatcctggcggagttctgagactgtcaggcggagcaggcgttattggcatgaaaacaggcggaacagcagttgcaattgttccgaaactggtttcaacagcaggcacagttgatgcatggcatacaggcacacagtattttagagcagatgattgggaaaatcttgcatcaggcacaggctttacagcactgaaagcagtcgcaggcagaacacttaaagtttcagttggcaaactgacaggctcaggcgaaacaagactgcatggcggaggcgcagttagactggatgttacagatggcgaaagatatctgggcgttgttagagtttcatcaggcgcagcagattttgataataacgtttttgtttcaggaccgctggttattgaaacaggcgctacagttgttctggatcaagcagtttcatttgcaggccttacagttgctggcacagaatattcaccgggaaattatacatttgcagcacttcaagcagcacatccgacggtttttacaagcggcacagcaggcggatcaattacagttagagcaccgagaacatggtatctgacagttaatcaaggcggagtccaaaattggacagaaacatatctgagcaattggaattcagcagcaaatggatcaggcgttgcaccgacatcaattaatggctatgacttttatatcgatcaggtcagcaatcgcgaaattagaacaccgtcaacagcatcaacatttggaggcggagcgctggcactggcatctggcgcaaaactgacactgaaatcatcacctggcgttgtttcaacaattccggcatttgttaatacaaacagcccgattattgttaatggcggtggctcatttagacaatcactggcacttggcgactgggaaattgcaagcggcattacaaaactgtcagcaggcagcggcagatcactgggctttgatattgattatcttggcggagctggcggactggttacacaaaatggcggatcatactttctgtcactggatgatggctcaggctatacgggcacactgaatcatgcgtcaggcgcactgagatttgaatcagtttttagcacagaaggcgcacttacaattggctcatcagcaacagttcatcttgatcaacaagtctatgtcacaagctttagcgttgcaggcgtcgcaaaagcagcaggcattcatacatatgcatcactgaatgcagcgcatccggcacaatttacagctggcgcagcaccgggactggttgcagtttatacaccggatacagcaggaccggttagaatgaatggcgtcaatattagcggaccggaatcaaatacagcaaatcttccgggaacatatggctataactatgtctatccgacagaagcggactttgattattatgcatcaaaaggcctgaacctgattagaattccgtttagatgggaaagaatgcagcatggcctgaatgttccgctgaatacagcacaactgggctatatggatacagcggttgcaagagcatcagcaagaggcatgaaagttattctggacatgcataactatgcacgctgcaaagttggaggcgttacatacaaatttggagatgcacaacttccggcaagcgcatatgcagatgtttggcgcagacttgcagaccactataaaaacgaaccggcaatttatggctttgacattatgaatgaaccgaatggcctgagcggaggcgtttggcctgcgtatgcacaagcagcagtcaatgcaattagagaagttaatctgagcacatgggttattgtcgaaggcgaattttgggcaaatgcatggggctttgaaacgaaaaatccgtatctgcataatgtgagagatccggttggcagactgatgttttcagcacattcatattggtcagatgcaggcacggatgtctataaaacatatgatgaagaaggcgcttatccggaaatgggcgttaataatgttaaaccgtttatcgattggctgaaaaaacatgacgcaaaaggctttgttggcgaatatggcgttccgaataatgatccgagatggctggttgtcctggataattttctggcatatctggcagcagaaggcgtttcaggcacatattgggctggcggagcatggtattcaggctcaccgattagctgccatccgtcaagcaactatacagttgatagagcagttatgagcgtcctggaagatcatccgtaa

SEQ ID NO: 10 - DNA, 2452 bp, Artificial
  Codon optimized
gcttttagttcatcgatagcatcagcacatcatcatcaccatcatccgagagcagattggtatctggataaaaatcaagcaagatatgcgagctgggatacactggcagattggaaaccgaatccggatggctcaggctcaaatccgtcagcactgtcaccgtcagatacatatcatctgaatggctttatgctgagaacaccggaaggcggatcaacatatacatttacaggcggactgctgagcctggcaaataatgcagataattttgcgctgaaaacaacaggctcaggcgtttcaattattccggcactgagaacaacagcaggcctggttcaaaatgttggcagcggcacacaaaatctgcaagttggccattatcaaaatctgtcaggcacaacaagctattatgcacaaacaggcagaggcctgaatctggcaattacaacactggttggctcaggacagtttagattttatggcggaggcacatattatctgtctctggcaaattcaccgacatatgatggcgatatttatgtccaaagcggcacaattgattttaacaatgatctggcgacagcaggcacactgacagttaatacaggcgcaaaagttgcactggatcaagcagttacgtttacaggactgacaattgcaggcacagcatatccggttggcaattattcatatgcagcactgcaagcagcacatccggcagtttttgtttctggcacatcaggcggagcaattaatgttagagcaccgagaaattggtacttgtcaacacatcagccggttggcgcatcatggaatacacttgcgcattggagagcaaacccggatggaacaggcgctacagcagattcaattaatagctttgacaactatatcaaccaggtcagcggcagaacactgcgcacaccggaaacaacagcgacatttgctggcggatcactggttctggcagatggcggaaatctttcactgaaagcaccggcaggccattcatcaacaattccggcatttgcaacatcaggcagcatttcaattacaaacggctttagctcaattacacaaccgctggttattggcgattggcatcttggcgctggcacagcacaagtttcagttccgtcaacatcaacagttcaactgacagtcgataaactgagcggagatggcacactgcaatttcaaaatggcggtaaatatacgctgaacattagaggcgcatcagcttttacaggcacattaagacatctgagcggaacacttacagttgcatcacaaattggcacaggcggaacattagttgttgaatcaacaggcgcagttaaactggatcatccgggattttttacaggtgttacagtggctggcacaccgctggcaccgggatatcatacatatgcggcacttaaagcggctcatcctgcgagatttccgacaggctcaacaaatgcgtttcttgcagtttatcctccggatacaacaggaccggcacatatgtttggcgttaatctggctggcggagaatttggaacaccgatgcctggcgtttatggcacagattatatctatccgagcgcagcagcatttgattattatcatggcaaaggccttaaactgattcgcctgccgtttaaatgggaaagactgcaacatacacttaatgcaccgctgaatgcagcagaactggcaagaattgatacagttgttggctatgcatcagcaagaggcatgaaagttgttctggatatgcataactatgcgcgtagaaaagaatcaggcacgacatatctgatcggcacaggccctgttacaatggatgcatttggagatgtttggagaagaatcgcggatcattataaaggcaatccggcaatttatggctacggcattatgaatgaaccgtatagcacaaatacaacgtggcctcaaatggcgcaaacagcagttaatgcaattagaacagttgatctgacaacgcatgttattgttgcaggcgacggctggtcaaatgcaacaggctggcgctcaaaaaatccgaatctggatacacaagatccggtcggcagactgatttatgaagcacattgctattttgacagcaacctttcaggcacgtatacacaaagctatgatgcagcaggcgcacatccgatgattggcgttgatagagttagagaatttgtcgaatggcttcaagaaacaggcaacaaaggctttattggagaatatggcgttccgggaaatgatccgagatggctggttgttcttgataattttctggcatatctggatgcaaatggcgttagcggaacatattgggcaggcggaccgtggtggggcaattatccgctgtcatgcgaaccgacatcaaattacacagttgataaaccgcaaatgagcgtcctggaaaactacaactaaacgcgttaatcaataa

SEQ ID NO: 11 - DNA, 2403 bp, Artificial
  Codon optimized
gcagattattatctgaaagttaatcaaccgcatccgaattcatgggcatcaccggttacagattgggcagcaaatccggatggcacaggcgcagcaccggcagcaattgcggcaccggatacattttatacaaataatagaacacttcgcacaccggctgttggcgttaatgcaacatttcctggcggagttctgggcctgaatggcggagtcattggcattaaaacaggaccgtcagcattttcaattgcaccgaaactggtttcaacagcaggcgcaattgaatcatggggcacaccgcagaattttagagcagatgattgggaatcaaatgcaccgtttccgacatttacaggcctgagaacagcatcaaatcatacacttaaagttagcgttggcaaactgagcggaacaggcgaaattagagttcatggcggaggcacagttctgctggatgttacagatgcagaaaattatctgggcacactgtgcgttgcatcaggcgcactgaattttgataatgcagttttttcatcaggaccgctggatatcaaaacaggcgcaacagttgttctggatcaagcagtttcatttgcaggccttgcagttggagcaacagaatatccgcctggcaattatacactggcagcactgcaagcagcacatcctggcgtttttacaggcacagcagcaggatcaattacagttagagcaccgagaacatggtatctgacagtttcacaaggctcacaaaattggacagaagcatttctgtcaaattggaattcagcagcaaatggctcaggcgtcgcaccgaattatatcaatggacatgatatctatctgaaccaggtcaataatcgcgaactgagaacaccgtatacagcaagcacgtttacaggcggaacactggcactgacatttggctcaaaactggttgttaaaacaagcccgaatctggttagcacaattccggcactggttacatctggaacaccgcaatttgcgaatggcagcggctcaagacaaaatctggcaattggcgattgggatattatctcaggcacatcaagactggttgcaggctcaacaagatcactgggctttgatattggctggctgacaggcgctggcaatctgcaaacagaaggcggaggctcattttttctgagactgattgatggatcaggctatacaggcgctattaaccataattctggcgctctgagatttgaaagcgtttttagcacagctggcgcacttaatattggcgcatcagcaacagttcatcttgataaaccggtctatgtttcaggccttagcgttgcaggcgttgcgaaaccggcaggcattcatacatatgcatcacttaatgcagcgcatccggcacaatttaatgcaggcgctgctccgggacttgttgcagtttatacaccgaacacagcagctccggttagaatgaatggcgtcaatctgtcaggaccggaatcagttggcggagcaggtacaccttttccgggaacatatggctttcaatggatttatccgacagtcgcggattatgattattatgcagcaaaaggccttaacctgattagaattccgtttagatgggaaagaatgcaaggcacactgaatggaccgctgattgcagcggaactggcaagaatggataatgcaattgcgctggcatcagcgagaggcatgaaagttattctggatatgcataactatgcacgctatagaacaccgacagcatcatatgtttttggagatgcgcaacttccggcatcagcatttgcagatgtttggagaaaactggcggatcactataaaaacgaaccggcaatttatggctttgacattatgaatgaaccgcattcaatgccgacaccgacaacgtggccgacatatgcacaagcagcagttcatgcaattagagaagtcaatctggatacatggattatcgttgaaggcgaaacatatgcgaactcatggaaatttggcgaaaaaaatccgcatctgcataatgttagagatccggttggcagactgatgttttcagcacattcatattggtgcaaaaatggcgacgatcgctatggcacgtatgatgcggaaaatggccatccgcaaatgggcgttgattcactgaaacattttgttgattggctgcgcaaacataatgcacatggctttgttggcgaatatggcgttccgaataatgatccgagatggctggaagttctggaaaatgcactgatttatctggcgaacgaaaacattagcggcacatattgggcaggcggagcatggctggcaggctcacatatttcatgccatccgtcatctaactatacagttgatcgtccggttatgagcgtcctgcaaaattatccgtaa

SEQ ID NO: 12 - DNA, 1974 bp, Artificial
  Codon optimized
tcatcagttgcagcagtttcagtttcagcaaaaatcaatgcgtttacgaatagcgattggctgaatggcatttggagaacaggcgcaggcttttcaattccggcaacatcagcaaatagagcagcatttgttgcaggcgcatcagttagactggcagatggccaagttagaaaaattagcagagcacaaattgtcggcagcaacatgtcaatttttctggaaggcgcaaaactggatggcaataaagttggcgcaccgcaagttgttacaattggctcaacagcagttacagcaccggatacatcagcaccgattacaacaccgcctacagtcacagcacattcaacatcaattaacgcctttacaaataatgactggcttaacggcgtttggcgcaaatcaccgggatttagcattccggcatctgcagcgaataaagcggcttttaaagttggagcaacagcaaaacttgcggatggacaggttcgcaaaattacacaagttcaagttgttggcgctaacatgagcgtttatcttgaaggcgcagcagtcaatggctcagttgttggagcaccgaataaactggcactggcaacaacaagcacaacatcaccggcaccgacaccggctccgtcagctccgacaccgtcagttattgcaacatcaaatctgaacaactatacaaatgcgcagtggctgaacggaatgtatagaacagcagcgggattttctattcaagcatcaagcgcaaatgtcgcagcatttaaagcaggcgcactggtcagacttgctgatggccagacaagaaaagttctgagagcacaactggttggctcaaatatgtcagtctttcttgatggcgctgtcattaatggcacaacactgggctatccgaaaacaatttcagttgttagcacatcaacaggcacaccgtcatctccggcactgacaacacctccggttgaaccggctcctgcaccggttccgacagcgcctgatacaacaaatggcaaaccgctgctggttggcgttaatctgagcggagcaggctttggaccgagcgttgttccgggaaaacatggcacaaattatacatatccggcagaaagctactacaaaaaatactcagatctgggcatgccgctggttagactgccgtttctgtgggaaagaattcaaccgaaactgaattcaccgctgaatgcagaagaatttgcaagactgaaacagagcctggattttgcgcagaaacataacgttaaagtcatcctggatctgcataactattatcgctattacggcaaactgattggcagcaaagaagttccgatttcaagctttgcggcagtctggaaacaaattgttcaacaagttgtcaatcatccggcagttgaaggctatggcctgatgaatgaaccgcatagcacaaatggcctgtggcctcaagcagcactggcagcagcacaagcaattagaacagttgatagcaaacgctggatttatgtcgcaggcgatagatggtcatcagcatttcattggcctcattataacacacagctggttacaaatccgtggatgagagatccgaaaaataacctggtttatgaagcgcatatgtatgtcgacaaagattttagcggcaactactttgacaaagcggaaaaatttgatccgatgattggcgtcaatcgcgttaaaccgtttgttgattggcttaaacagcataaactgcgtggctatattggcgaacatggcgttccggatttttcaccgtcagcaattgttgcgacagataatctgctggcatatctgagacaaaattgcattccgtcaacatattgggcagcaggaccgtggtggggagaatatgcaatgtcactggatgtttcaagcggcaaacatagaccgcaacttccggttcttcaaaaacatgcaaaaacagcgaatagctgcacatcaattggaccgctgtaa

SEQ ID NO: 13 - protein, 811 aa, Artificial
  HISTAG′ed
  MISC_FEATURE (1)..(9): His tag
MHHHHHHPRADYYLKASQGASNHWSSHLTDWTANADGTGANPTVIGLADTFDTNNRTLRTPAVNATTTYPGGVLRLSGGAGVIGMKTGGTAVAIVPKLVSTAGTVDAWHTGTQYFRADDWENLASGTGFTALKAVAGRTLKVSVGKLTGSGETRLHGGGAVRLDVTDGERYLGVVRVSSGAADFDNNVFVSGPLVIETGATVVLDQAVSFAGLTVAGTEYSPGNYTFAALQAAHPTVFTSGTAGGSITVRAPRTWYLTVNQGGVQNWTETYLSNWNSAANGSGVAPTSINGYDFYIDQVSNREIRTPSTASTFGGGALALASGAKLTLKSSPGVVSTIPAFVNTNSPIIVNGGGSFRQSLALGDWEIASGITKLSAGSGRSLGFDIDYLGGAGGLVTQNGGSYFLSLDDGSGYTGTLNHASGALRFESVFSTEGALTIGSSATVHLDQQVYVTSFSVAGVAKAAGIHTYASLNAAHPAQFTAGAAPGLVAVYTPDTAGPVRMNGVNISGPESNTANLPGTYGYNYVYPTEADFDYYASKGLNLIRIPFRWERMQHGLNVPLNTAQLGYMDTAVARASARGMKVILDMHNYARCKVGGVTYKFGDAQLPASAYADVWRRLADHYKNEPAIYGFDIMNEPNGLSGGVWPAYAQAAVNAIREVNLSTWVIVEGEFWANAWGFETKNPYLHNVRDPVGRLMFSAHSYWSDAGTDVYKTYDEEGAYPEMGVNNVKPFIDWLKKHDAKGFVGEYGVPNNDPRWLVVLDNFLAYLAAEGVSGTYWAGGAWYSGSPISCHPSSNYTVDRAVMSVLEDHP

SEQ ID NO: 14 - protein, 803 aa, Artificial
  His tag′ed
  MISC_FEATURE (1)..(9): His tag
MHHHHHHPRADWYLDKNQARYASWDTLADWKPNPDGSGSNPSALSPSDTYHLNGFMLRTPEGGSTYTFTGGLLSLANNADNFALKTTGSGVSIIPALRTTAGLVQNVGSGTQNLQVGHYQNLSGTTSYYAQTGRGLNLAITTLVGSGQFRFYGGGTYYLSLANSPTYDGDIYVQSGTIDFNNDLATAGTLTVNTGAKVALDQAVTFTGLTIAGTAYPVGNYSYAALQAAHPAVFVSGTSGGAINVRAPRNWYLSTHQPVGASWNTLAHWRANPDGTGATADSINSFDNYINQVSGRTLRTPETTATFAGGSLVLADGGNLSLKAPAGHSSTIPAFATSGSISITNGFSSITQPLVIGDWHLGAGTAQVSVPSTSTVQLTVDKLSGDGTLQFQNGGKYTLNIRGASAFTGTLRHLSGTLTVASQIGTGGTLVVESTGAVKLDHPGFFTGVTVAGTPLAPGYHTYAALKAAHPARFPTGSTNAFLAVYPPDTTGPAHMFGVNLAGGEFGTPMPGVYGTDYIYPSAAAFDYYHGKGLKLIRLPFKWERLQHTLNAPLNAAELARIDTVVGYASARGMKVVLDMHNYARRKESGTTYLIGTGPVTMDAFGDVWRRIADHYKGNPAIYGYGIMNEPYSTNTTWPQMAQTAVNAIRTVDLTTHVIVAGDGWSNATGWRSKNPNLDTQDPVGRLIYEAHCYFDSNLSGTYTQSYDAAGAHPMIGVDRVREFVEWLQETGNKGFIGEYGVPGNDPRWLVVLDNFLAYLDANGVSGTYWAGGPWWGNYPLSCEPTSNYTVDKPQMSVLENYN

SEQ ID NO: 15 - protein, 809 aa, Artificial
  HISTAG′ed
  MISC_FEATURE (1)..(9): His tag
MHHHHHHPRADYYLKVNQPHPNSWASPVTDWAANPDGTGAAPAAIAAPDTFYTNNRTLRTPAVGVNATFPGGVLGLNGGVIGIKTGPSAFSIAPKLVSTAGAIESWGTPQNFRADDWESNAPFPTFTGLRTASNHTLKVSVGKLSGTGEIRVHGGGTVLLDVTDAENYLGTLCVASGALNFDNAVFSSGPLDIKTGATVVLDQAVSFAGLAVGATEYPPGNYTLAALQAAHPGVFTGTAAGSITVRAPRTWYLTVSQGSQNWTEAFLSNWNSAANGSGVAPNYINGHDIYLNQVNNRELRTPYTASTFTGGTLALTFGSKLVVKTSPNLVSTIPALVTSGTPQFANGSGSRQNLAIGDWDIISGTSRLVAGSTRSLGFDIGWLTGAGNLQTEGGGSFFLRLIDGSGYTGAINHNSGALRFESVFSTAGALNIGASATVHLDKPVYVSGLSVAGVAKPAGIHTYASLNAAHPAQFNAGAAPGLVAVYTPNTAAPVRMNGVNLSGPESVGGAGTPFPGTYGFQWIYPTVADYDYYAAKGLNLIRIPFRWERMQGTLNGPLIAAELARMDNAIALASARGMKVILDMHNYARYRTPTASYVFGDAQLPASAFADVWRKLADHYKNEPAIYGFDIMNEPHSMPTPTTWPTYAQAAVHAIREVNLDTWIIVEGETYANSWKFGEKNPHLHNVRDPVGRLMFSAHSYWCKNGDDRYGTYDAENGHPQMGVDSLKHFVDWLRKHNAHGFVGEYGVPNNDPRWLEVLENALIYLANENISGTYWAGGAWLAGSHISCHPSSNYTVDRPVMSVLQNYP

SEQ ID NO: 16 - protein, 802 aa, Artificial
  HISTAG′ed
  MISC_FEATURE (1)..(8)
  MISC_FEATURE (1)..(8): Has tag
HHHHHHPRADWYLDKNQARYASWDTLADWKPNPDGSGSNPSALSPSDTYHLNGFMLRTPEGGSTYTFTGGLLSLANNADNFALKTTGSGVSIIPALRTTAGLVQNVGSGTQNLQVGHYQNLSGTTSYYAQTGRGLNLAITTLVGSGQFRFYGGGTYYLSLANSPTYDGDIYVQSGTIDFNNDLATAGTLTVNTGAKVALDQAVTFTGLTIAGTAYPVGNYSYAALQAAHPAVFVSGTSGGAINVRAPRNWYLSTHQPVGASWNTLAHWRANPDGTGATADSINSFDNYINQVSGRTLRTPETTATFAGGSLVLADGGNLSLKAPAGHSSTIPAFATSGSISITNGFSSITQPLVIGDWHLGAGTAQVSVPSTSTVQLTVDKLSGDGTLQFQNGGKYTLNIRGASAFTGTLRHLSGTLTVASQIGTGGTLVVESTGAVKLDHPGFFTGVTVAGTPLAPGYHTYAALKAAHPARFPTGSTNAFLAVYPPDTTGPAHMFGVNLAGGEFGTPMPGVYGTDYIYPSAAAFDYYHGKGLKLIRLPFKWERLQHTLNAPLNAAELARIDTVVGYASARGMKVVLDMHNYARRKESGTTYLIGTGPVTMDAFGDVWRRIADHYKGNPAIYGYGIMNEPYSTNTTWPQMAQTAVNAIRTVDLTTHVIVAGDGWSNATGWRSKNPNLDTQDPVGRLIYEAHCYFDSNLSGTYTQSYDAAGAHPMIGVDRVREFVEWLQETGNKGFIGEYGVPGNDPRWLVVLDNFLAYLDANGVSGTYWAGGPWWGNYPLSCEPTSNYTVDKPQMSVLENYN

SEQ ID NO: 17 - protein, 808 aa, Artificial
  HASTAG′ed
  MISC_FEATURE (1)..(8): Has tag
HHHHHHPRADYYLKVNQPHPNSWASPVTDWAANPDGTGAAPAAIAAPDTFYTNNRTLRTPAVGVNATFPGGVLGLNGGVIGIKTGPSAFSIAPKLVSTAGAIESWGTPQNFRADDWESNAPFPTFTGLRTASNHTLKVSVGKLSGTGEIRVHGGGTVLLDVTDAENYLGTLCVASGALNFDNAVFSSGPLDIKTGATVVLDQAVSFAGLAVGATEYPPGNYTLAALQAAHPGVFTGTAAGSITVRAPRTWYLTVSQGSQNWTEAFLSNWNSAANGSGVAPNYINGHDIYLNQVNNRELRTPYTASTFTGGTLALTFGSKLVVKTSPNLVSTIPALVTSGTPQFANGSGSRQNLAIGDWDIISGTSRLVAGSTRSLGFDIGWLTGAGNLQTEGGGSFFLRLIDGSGYTGAINHNSGALRFESVFSTAGALNIGASATVHLDKPVYVSGLSVAGVAKPAGIHTYASLNAAHPAQFNAGAAPGLVAVYTPNTAAPVRMNGVNLSGPESVGGAGTPFPGTYGFQWIYPTVADYDYYAAKGLNLIRIPFRWERMQGTLNGPLIAAELARMDNAIALASARGMKVILDMHNYARYRTPTASYVFGDAQLPASAFADVWRKLADHYKNEPAIYGFDIMNEPHSMPTPTTWPTYAQAAVHAIREVNLDTWIIVEGETYANSWKFGEKNPHLHNVRDPVGRLMFSAHSYWCKNGDDRYGTYDAENGHPQMGVDSLKHFVDWLRKHNAHGFVGEYGVPNNDPRWLEVLENALIYLANENISGTYWAGGAWLAGSHISCHPSSNYTVDRPVMSVLQNYP

SEQ ID NO: 18 - protein, 665 aa, Artificial
  HASTAG′ed
  MISC_FEATURE (1)..(8): Has tag
HHHHHHPRSSVAAVSVSAKINAFTNSDWLNGIWRTGAGFSIPATSANRAAFVAGASVRLADGQVRKISRAQIVGSNMSIFLEGAKLDGNKVGAPQVVTIGSTAVTAPDTSAPITTPPTVTAHSTSINAFTNNDWLNGVWRKSPGFSIPASAANKAAFKVGATAKLADGQVRKITQVQVVGANMSVYLEGAAVNGSVVGAPNKLALATTSTTSPAPTPAPSAPTPSVIATSNLNNYTNAQWLNGMYRTAAGFSIQASSANVAAFKAGALVRLADGQTRKVLRAQLVGSNMSVFLDGAVINGTTLGYPKTISVVSTSTGTPSSPALTTPPVEPAPAPVPTAPDTTNGKPLLVGVNLSGAGFGPSVVPGKHGTNYTYPAESYYKKYSDLGMPLVRLPFLWERIQPKLNSPLNAEEFARLKQSLDFAQKHNVKVILDLHNYYRYYGKLIGSKEVPISSFAAVWKQIVQQVVNHPAVEGYGLMNEPHSTNGLWPQAALAAAQAIRTVDSKRWIYVAGDRWSSAFHWPHYNTQLVTNPWMRDPKNNLVYEAHMYVDKDFSGNYFDKAEKFDPMIGVNRVKPFVDWLKQHKLRGYIGEHGVPDFSPSAIVATDNLLAYLRQNCIPSTYWAAGPWWGEYAMSLDVSSGKHRPQLPVLQKHAKTANSCTSIGPL

SEQ ID NO: 19 - protein, 8 aa, Artificial
  Has tag
HHHHHHPR

SEQ ID NO: 20 - protein, 27 aa, Bacillus clausii
MKKPLGKIVASTALLISVAFSSSIASA

SEQ ID NO: 21 - protein, 795 aa, Paenibacillus sp
  mat_peptide (28)..(795)
MKKPLGKIVASTALLISVAFSSSIASAHHHHHHPRAEASDMFDELREKYATMLTGGTAYSLSDPDIAARVASITTNAQTLWTSMKKDANRVRLWDNAPLGNDSASITTSYRQLAAMALAYRTYGSSLMGDPDLRDDIIDGLDWINTFQHGFCEGCSMYQNWWHWQIGGPIALNEVIALMYDELTQTQIDSYIAAINYAQPSVNMTGANRLWESQVIALAGINGKNGDKIAHARDGLSALLTYVVQGDGFYEDGSFVQHSYYSYNGGYGLDLLKGIADLTYLLHDSNWEVVDPNKQNIFNWVYDSFEPFIYNGNLMDMVRGREISRHARQSNVVGVEAVAAILRLSHVAPPADAAAFKSMVKHWLQEGGGSQFLQQASITHILSAQDVLNDSGIVPRGELEAYRQFAGMDRALQLRQGYGFGISMFSSRIGGHEAINAENNKGWHTGAGMTYLYNNDLSQFNDHFWPTVNSYRLPGTTVLRDTPQAANTRGDRSWAGGTDMLGLYGITGMEYHAIGKSLTAKKSWFMFDDEIVALGADITSGDGVAVETIVENRKLNGAGDNSLTVNGTAKPATLGWSETMGTTSYAHLGGSVADSDIGYYFPDGGATLHALREARTGNWRQINSAQGSPNAPHTRNYLTMWLEHGVNPSNGAYSYVLLPNKTSAATASYAASPDITIIENSSSAQAVKENGLNMIGVNFWNNERKTAGGITSNAKASVMTRETASELNVSVSDPTQSNVGMIYIEIDKSATGLIAKDDAVTVLQYSPTIKFKVDVNKARGKSFKAAFSLTGAQQP

SEQ ID NO: 22 - protein, 1073 aa, Paenibacillus sp
  mat_peptide (28)..(1973)
MKKPLGKIVASTALLISVAFSSSIASAHHHHHHPRADEFDTLREKYKAMLNGGTTYNLSDPDIAARVNAITVTAQGYWDSMLKDPNRNRLWNDAPFGSDSTSITTTYRHLYDMALAYTTYGSSLQGNAALKADIISGLDWMNANQFYNGCSQYQNWWHWQIGGPMALNDIVALMYTELTATQISNYMAAIYYTQASVTMTGANRLWESQVIAISGILNKDSARVAAGRDGISALLPYVAKGDGFYNDGSFVQHTYYAYNGGYGSELLSGIADLIFILNGSSWQVTDPNKNNVYRWIYDSYEPFIYKGNLMDMVRGREISRHGLQDDKAAVTVMASIIRLSQTAASADATAFKRMVKYWLLLDTDKTFLKAVSIDLIIAANQLVNDSTVTSRGELVKYKQFSGMDRAVQLRPGFGFGLSMFSSRIGNYESINAENNKGWHTGDGMTYLYNTDLSQFNDHFWATVDNYRLPGTTVLQNTTQTANSRSDKSWAGGTDILGQYGVSGMELHTVGKSLTAKKSWFMFDDEIVALGSGIASTDGIATETIVENRKLNSSGNNALIVNGTAKPGSLGWSETMTGTNYIHLAGSVPGSDIGYYFPGGAAVKGLREARSGSWSSLNSSASWKDSTLHTRNFMTLWFDHGMNPTNGSYSYVLLPNKTSSAVASYAATPQISILENSSSAQAVKETQLNVTGINFWNDEPTTVGLVTSNRKASVMTKETASDFEISVSDPTQSNVGTIYIDVNKSATGLISKDNEITVIQYYPTMKFKVNVNNSGGKSYKVKFSLTGTPGSNPSPIPIPNPYEAEALPINALTDTPVVYNDANASGGKKLGFNNNAVDDYVEFSLDVTQPGTYDVKSRIMKSTNSGIYQLSINGTNVGSAQDMFWTTSELSKEFTMGSYSFSTPGSYLFRLKTTGKNVSSSGYKLMLDNFSLVSTGIDTTVIVDNADAAGVTKVGTWTGTNTQTDRYGADYIHDGNTGKGTKSVTFTPNVPISGTYQVYMMWAAHTNRATNVPVDVTHSGGTATLNVNQQGNGGVWNLLGTYSFNAGSTGAIKIRTDATNGYVVADAVKLVKVP

SEQ ID NO: 23 - protein, 1078 aa, Paenibacillus sp
  mat_peptide (28)..(1078)
MKKPLGKIVASTALLISVAFSSSIASAHHHHHHPRAEASDMFDELREKYATMLTGGTAYSLSDPDIAARVASITTNAQTLWTSMKKDANRVRLWDNAPLGNDSASITTSYRQLAAMALAYRTYGSSLMGDPDLRDDIIDGLDWINTFQHGFCEGCSMYQNWWHWQIGGPIALNEVIALMYDELTQTQIDSYIAAINYAQPSVNMTGANRLWESQVIALAGINGKNGDKIAHARDGLSALLTYVVQGDGFYEDGSFVQHSYYSYNGGYGLDLLKGIADLTYLLHDSNWEVVDPNKQNIFNWVYDSFEPFIYNGNLMDMVRGREISRHARQSNVVGVEAVAAILRLSHVAPPADAAAFKSMVKHWLQEGGGSQFLQQASITHILSAQDVLNDSGIVPRGELEAYRQFAGMDRALQLRQGYGFGISMFSSRIGGHEAINAENNKGWHTGAGMTYLYNNDLSQFNDHFWPTVNSYRLPGTTVLRDTPQAANTRGDRSWAGGTDMLGLYGITGMEYHAIGKSLTAKKSWFMFDDEIVALGADITSGDGVAVETIVENRKLNGAGDNSLTVNGTAKPATLGWSETMGTTSYAHLGGSVADSDIGYYFPDGGATLHALREARTGNWRQINSAQGSPNAPHTRNYLTMWLEHGVNPSNGAYSYVLLPNKTSAATASYAASPDITIIENSSSAQAVKENGLNMIGVNFWNNERKTAGGITSNAKASVMTRETASELNVSVSDPTQSNVGMIYIEIDKSATGLIAKDDAVTVLQYSPTIKFKVDVNKARGKSFKAAFSLTGAQQPNPAPIPIPNPYEAELLPISATTKTPTLSNDSNASGGKKLGLNSSVVGDYTEFSLDVTQPGTYDIAAKIMKVSNNGIYQFSINGEPVGDPVDMYWNTSESTKSFSPGSYTFSEPGSYLLRVTVTGKHPSSSGYKLMLDHFTLEEIPVSLPNPYEAETLPIHHRTQTVTIYNDSNTSGGQRLGLNHKVVGDYTEFILDVPQAGTYDITARVLKFSDNGIYQFSIDGNPVGAPIDTYWNTAGYIRDFTPGSYTFSEPGSYLLRLTATGKNPSASGLKIMLDYIWLD

SEQ ID NO: 24 - protein, 968 aa, Paenibacillus sp
  mat_peptide (28)..(968)
MKKPLGKIVASTALLISVAFSSSIASAHHHHHHPRGGEASGSADDAAETAEAAEGENIEDKMVSAYNMDAFDIMREVRRTMLTGGAALNPADPDAAAAVAALASEANQYWQTMDDSPGRTSLWSDNPGTGNSIHIRITYERLKTMALAYAAAGSPLHSNASLEADIVDALDYMYATRYHENVTTTPSGTSNWWDWQIGIPMQLNDTVVLMYDSLTPAQIANYMNAVERFTPTVNLTGANRSWKAIVVAVRGILVKDGAKIAAARDGLSQIFNYAVSGDGFYRDGSFIQHGNIPYNGGYGLDLLLAVSDLMTLLHGSAWQVTDPNQANVWEWVYRAYQPLIYKGAMMDMVRGREISRVYRQDHAAGHIAMQGILRLSAVAPPAQAEDFKRMVKGWMVVDGFMRFYEQAPLGLIPLAKAVEGDASIAPASELIQYRQYAAMDRAVQLRPGYGFGLAMYSSRIGSFEAINSENLRGWYTSAGMTSLYNGDLGHYSEDYWPTVNAYRLPGTTVLSGTAAASHTSPNNWTGGTDMQGLYGVSGMDLKYASNSLAARKSWFMFDDEIVALGAGISSADGIPVETIIENRRIGGAGDNAFLADGAAMPAELGWSGTLEGVRWAHLTGTAAGADIGYYFPEPAAVHAVREARTGNWRQINNRPVTPAASVTRNYLTFWFDHGANPTNADYQYVLLPNKSGAQVAGYAANPDVEVLANSPEVQAVKESSLGIIGANFWSDGVRTVDLITVNKKASVMTRETPGAILDLSVSDPTQVNAGTIEIELNRAASGFTADPGVTVTRLSPTIKLTVQVAGAKGRSFKASFELGEASGPGPDPGPGPSEIIVDNGDAAGVTKIGSWKTGTVQTDRYGPDYLHDDNTGKGGKSVRFTPDLPTAGTYDVYMMWPQHFNRATNIPVTIAHAGGTATVTIDQTVSGGVWNYLGSYSFDTGSGGSVTISNAGTNGYVVADAVKFEYVP